(19) United States
(12) Patent Application Publication
(10) Pub. No.: US 2024/0277900 A1
(43) Pub. Date: Aug. 22, 2024

(54) ENGINEERED TISSUE CONSTRUCTS AND USES THEREOF

(71) Applicant: Satellite Biosciences, Inc., Cambridge, MA (US)

(72) Inventors: Arnav CHHABRA, Mansfield, MA (US); Amanda CHEN, Cambridge, MA (US); Keval N. VYAS, Somerville, MA (US); Thomas Jay LOWERY, JR., Sudbury, MA (US); Fabiola MUNARIN, Smithfield, RI (US); Glenn Michael LA MURAGLIA, II, Boston, MA (US); Timothy Alfred NORMAN, JR., Dedham, MA (US); Christopher Garrison WILSON, Auburndale, MA (US); Joseph E. MARTURANO, Bedford, MA (US); Morgan JAMIEL, Somerville, MA (US); Alison HAYWARD, North Andover, MA (US); Howard TAKIFF, La Jolla, CA (US)

(21) Appl. No.: 18/646,092

(22) Filed: Apr. 25, 2024

Related U.S. Application Data
(63) Continuation-in-part of application No. PCT/US2022/047749, filed on Oct. 25, 2022.
(63) Continuation-in-part of application No. PCT/US2022/047730, filed on Oct. 25, 2022.
(63) Continuation-in-part of application No. PCT/US2022/047739, filed on Oct. 25, 2022.
(60) Provisional application No. 63/271,602, filed on Oct. 25, 2021.
(60) Provisional application No. 63/271,662, filed on Oct. 25, 2021.
(60) Provisional application No. 63/271,615, filed on Oct. 25, 2021.

(51) Int. Cl. A61L 27/38; A61F 2/02; A61L 27/22; A61L 27/44; A61L 27/52; A61L 27/54

(52) U.S. Cl. CPC A61L 27/3839; A61F 2/022; A61L 27/225; A61L 27/3813; A61L 27/44; A61L 27/52; A61L 27/54; A61L 2300/412; A61L 2430/28

(57) ABSTRACT

The present disclosure provides engineered tissue constructs having hepatocytes and stromal cells and methods of making and using the same (e.g., for treating acute liver failure, a urea cycle disorder, or hyperbilirubinemia (e.g., in a subject having Crigler-Najjar syndrome) in a human subject in need thereof). The engineered tissue constructs may provide a microenvironment that promotes the persistence of hepatocyte survival for at least three months.

### FIELD OF THE INVENTION

The present disclosure concerns the use of engineered tissue constructs and methods of making and using the same (e.g., for treating acute liver failure, a urea cycle disorder, hyperbilirubinemia, or Crigler-Najjar syndrome) in a human subject in need thereof).

### BACKGROUND OF THE INVENTION

Liver failure is the inability of the liver to perform its normal synthetic and metabolic function as part of normal physiology. Acute liver failure can occur in as little as 48 hours, and typically coincides with the loss or dysfunction of 80-90% of liver cells. Liver failure is a life-threatening condition that demands urgent medical care. Acute liver failure has an estimated prevalence of 2000 cases per year and a mortality rate of approximately 80 percent. In many cases, orthotopic liver transplantation is the only effective treatment for acute liver failure. The use of such transplants, however, is limited due to donor shortages, high cost, and the requirement for life-long immunosuppression. Thus, there is a clear need for alternative treatments for the treatment of liver failure.

Many diseases result from damage, malfunction, or loss of a single organ or tissue type. While certain strategies such as organ transplants can be effective, the demand for replacement organs far exceeds availability, resulting in an average of 18 deaths per day in the United States, alone. Tissue therapeutics, including the development of engineered tissue constructs (e.g., cell-based implants), are among the most promising multidisciplinary approaches to fulfill this demand. However, despite significant advances in the fields of cell biology, microfluidics, and engineering, to date, conventional approaches have failed to re-create functional tissues at a scale necessary to impart therapeutic efficacy. Current approaches in tissue engineering have relied on the in-growth of blood vessels into engineered tissues to achieve functional vascularization. This strategy has worked for some tissues that are either very thin, such as an engineered bladder wall, or tissues such as hypovascular tissues (e.g., cartilage) that do not require vasculature to function. However, current tissue engineering techniques fall short in the creation of complex tissues such as large vital organs, including the liver, kidney, thick skin, and heart. In particular, the formation of large tissue constructs with sufficient cell mass to replace critical organ functions can be hampered by the diffusion limit of oxygen and nutrients to cells within the construct. As such, certain efforts to implant large, engineered tissue constructs are hindered by an inability to deliver cell mass of therapeutic value. Therefore, there exists an unmet need for engineered tissues that can vascularize in vivo and support the function of high-density cell masses.

Crigler-Najjar syndrome is an autosomal recessive disorder of bilirubin metabolism that is caused by a variety of alterations in the coding sequence of the uridine diphosphate glucuronosyl transferase 1A1 (UGT1A1) gene. The total loss of UGT1A1 activity and the resulting severe jaundice and risk of neurological sequelae are associated with Crigler-Najjar syndrome type I. In contrast, Crigler-Najjar syndrome type II is characterized by unconjugated hyperbilirubinemia due to reduced and inducible activity of hepatic bilirubin glucuronosyltransferase. Although several drugs can slightly reduce jaundice, most current medical management relies on phototherapy for at least 12 hours per day. However, phototherapy rapidly becomes less effective following puberty, increasing the risk for kernicterus. When these treatment options fail, a liver transplant may be required. Additionally, since these patients sometimes require liver transplantation by the age of 10-13, multiple transplants may be required throughout the course of their lives. Thus, there is significant unmet need for effective, reliable, and long-term treatment for Crigler-Najjar syndrome.

### SUMMARY OF THE INVENTION

The present disclosure provides compositions and methods that can be used for treating acute liver failure (ALF) in a human subject in need thereof. Using the compositions and methods of the disclosure, a human subject having ALF may be administered an engineered tissue construct including a population of hepatocytes and optionally a population of stromal cells (e.g., fibroblasts).

In one aspect, the disclosure provides a method of treating ALF in a human subject in need thereof, the method including implanting an engineered tissue construct including a population of hepatocytes and optionally a population of stromal cells (e.g., fibroblasts) wherein the engineered tissue construct provide a microenvironment that promotes the persistence of hepatocyte survival for at least three months (e.g., at least three months, at least four months, at least five months, at least six months, at least one year, at least five years, or at least ten years) in the subject.

In some embodiments of the foregoing aspect, the population of hepatocytes includes an amount of hepatocytes that is equivalent to 0.5% to 30% (e.g., 0.5% to 30%, 0.6% to 30%, 0.7% to 30%, 0.8% to 30%, 0.9% to 30%, 1% to 30%, 2% to 30%, 3% to 30%, 4% to 30%, 5% to 30%, 10% to 30%, or 20% to 30%) of the total liver mass of the subject.

In some embodiments, the population of hepatocytes includes an amount of hepatocytes that is equivalent to 0.5% to 20% (e.g., 0.5% to 20%, 0.6% to 20%, 0.7% to 20%, 0.8% to 20%, 0.9% to 20%, 1% to 20%, 2% to 20%, 3% to 20%, 4% to 20%, 5% to 20%, or 10% to 20%) of the mass of the liver reserve of the subject.

In some embodiments of any of the foregoing aspects, the population of hepatocytes includes 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8× 10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes.

In some embodiments of any of the foregoing aspects, the optional population of stromal cells (e.g., fibroblasts) includes up to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², from about 3×10⁶ to about 1.8×10¹², from about 4×10⁶ to about 1.8×10¹², from about 5×10⁶ to about 1.8×10¹², from about 6×10⁶ to about 1.8×10¹², from about 7×10⁶ to about 1.8×10¹², from about 8×10⁶ to about 1.8×10¹², from about 9×10⁶ to about 1.8×10¹², from about 1×10⁷ to about 1.8×10¹², from about 2×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts).

In some embodiments, the hepatocytes are primary human hepatocytes. In some embodiments, the hepatocytes are derived from stem cells (e.g., induced pluripotent stem cells).

In some embodiments, the optional stromal cells are fibroblasts. In some embodiments, the fibroblasts are selected from the group consisting of normal human dermal fibroblasts and neonatal foreskin fibroblasts. For example, in some embodiments, the fibroblasts are neonatal foreskin fibroblasts.

In some embodiments, the ratio of hepatocytes to stromal cells (e.g., fibroblasts) is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

In some embodiments of any of the foregoing aspects, the implant is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). In some embodiments, the density of hepatocytes is 3 M/mL to 12 M/mL.

In some embodiments, the engineered tissue construct further includes a biocompatible hydrogel scaffold. For example, in some embodiments, the biocompatible scaffold includes fibrin. In some embodiments, the biocompatible scaffold includes heparin. In some embodiments, the heparin is a synthetic heparin mimetic.

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the omentum is the pedicled omentum. In some embodiments, the mesentery is the small intestinal mesentery. In some embodiments, the implant is implanted into the subject as a pedicled omental wrap or an omental wrap. In some embodiments, the implantation site is a site that is suitable for neovascularization.

In some embodiments, the engineered tissue construct is implanted into the subject at an implantation site that has a microvessel density of greater than about 3.6 vessels/mm² (e.g., greater than about 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

In some embodiments, the subject is a human.

In some embodiments, following implantation of the engineered tissue construct, the subject exhibits a level of serum ammonia of less than or equal to about 90 μmol/L (e.g., less than about 89 μmol/L, 88 μmol/L, 87 μmol/L, 86 μmol/L, 85 μmol/L, 84 μmol/L, 83 μmol/L, 82 μmol/L, 81 μmol/L, 80 μmol/L, 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L).

In some embodiments, following implantation of the engineered tissue construct, the subject exhibits a change in one or more parameters in a blood test relative to a reference level. In some embodiments, the blood test is a liver function test. For example, in some embodiments, the one or more parameters includes the level of gamma-glutamyl transferase, alkaline phosphatase, aspartate aminotransferase, alanine aminotransferase, or albumin.

In some embodiments, following implantation of the engineered tissue construct, the subject exhibits an improvement in a test of gallbladder ejection fraction. For example, in some embodiments, the test is a hepatobiliary iminodiacetic acid scan.

In some embodiments, the human subject weighs less than 15 kg (e.g., less than 15 kg, less than 14 kg, less than 13, kg, less than 12 kg, less than 11 kg, less than 10 kg, less than 9 kg, less than 8 kg, less than 7 kg, less than 6 kg, less than 5 kg, less than 4 kg, or less than 3 kg).

In another aspect, the disclosure provides a kit including an engineered tissue construct, wherein the kit further includes a package insert instructing a user of the kit to implant the engineered tissue construct to the subject in accordance with the method of any one of the foregoing embodiments.

The present disclosure also provides engineered tissue constructs and methods of making and using the same.

In another aspect, the disclosure provides an engineered tissue construct suitable for implantation into a subject, wherein the engineered tissue construct includes a population of hepatocytes and optionally a population of stromal cells (e.g., fibroblasts), wherein the hepatocytes and stromal cells are in a biocompatible scaffold, and wherein the population of hepatocytes and the optional population of stromal cells together account for at least 70% (e.g., at least 75%, 80%, 85%, 90%, 95%, or 100%) of the total cells in the engineered tissue construct.

In another aspect, the disclosure provides a method of treating liver dysfunction (e.g., acute liver failure, a urea cycle disorder, or Crigler-Najjar syndrome) in a human subject in need thereof. This method includes the step of implanting an engineered tissue construct including a population of hepatocytes and optionally a population of stromal cells (e.g., fibroblasts), wherein the hepatocytes and stromal cells are in a biocompatible scaffold, and wherein the population of hepatocytes and the optional population of stromal cells together account for at least 70% (e.g., at least 75%, 80%, 85%, 90%, 95%, or 100%) of the total cells in the engineered tissue construct.

In some embodiments of either of the foregoing aspects, the engineered tissue construct further includes less than 30% (e.g., less than 25%, 20%, 15%, 10%, or 5%) of other cell types (e.g., endothelial cells).

In another aspect, the disclosure provides an engineered tissue construct suitable for implantation into a subject, wherein the engineered tissue construct includes a population of hepatocytes and optionally a population of stromal cells (e.g., fibroblasts), wherein the hepatocytes and stromal cells are in a biocompatible scaffold, and wherein the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL).

In another aspect, the disclosure provides a method of treating liver dysfunction (e.g., acute liver failure, a urea cycle disorder, or hyperbilirubinemia (e.g., in a subject having Crigler-Najjar syndrome)) in a human subject in need thereof, the method includes implanting one or more engineered tissue constructs including a population of hepatocytes and optionally a population of stromal cells (e.g., fibroblasts), wherein the hepatocytes and stromal cells are in a biocompatible scaffold, and wherein the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). In another aspect, the disclosure provides an engineered tissue construct suitable for implantation into a subject, wherein the engineered tissue construct includes a population of hepatocytes and optionally a population of stromal cells (e.g., fibroblasts); wherein the hepatocytes and stromal cells are in a biocompatible scaffold; wherein the biocompatible scaffold has an x-axis, a y-axis, and a z-axis; and wherein the hepatocytes and stromal cells are distributed non-homogenously along the z-axis of the biocompatible scaffold. In some embodiments, the population of hepatocytes and the optional population stromal cells are distributed homogenously along the x-axis of the biocompatible scaffold. In some embodiments, the population of hepatocytes and the optional population stromal cells are distributed homogenously along the y-axis of the biocompatible scaffold.

In another aspect, the disclosure provides a method of treating liver dysfunction (e.g., acute liver failure, a urea cycle disorder, or hyperbilirubinemia (e.g., in a subject having Crigler-Najjar syndrome)) in a human subject in need thereof, the method includes implanting one or more engineered tissue constructs including a population of hepatocytes and optionally a population of stromal cells (e.g., fibroblasts); wherein the hepatocytes and stromal cells are in a biocompatible scaffold; wherein the biocompatible scaffold has an x-axis, a y-axis, and a z-axis; and wherein the hepatocytes and stromal cells are distributed non-homogenously along the z-axis of the biocompatible scaffold. In some embodiments, the population of hepatocytes and the optional population stromal cells are distributed homogenously along the x-axis of the biocompatible scaffold. In some embodiments, the population of hepatocytes and the optional population stromal cells are distributed homogenously along the y-axis of the biocompatible scaffold.

In another aspect, the disclosure provides an engineered tissue construct suitable for implantation into a subject, wherein the engineered tissue construct includes a plurality of spheroids in a biocompatible scaffold; wherein the spheroids include a population of hepatocytes and optionally a population of stromal cells (e.g., fibroblasts); wherein the biocompatible scaffold has an x-axis, a y-axis, and a z-axis; and wherein the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

In another aspect, the disclosure provides a method of treating liver dysfunction (e.g., acute liver failure, a urea cycle disorder, or hyperbilirubinemia (e.g., in a subject having Crigler-Najjar syndrome)) in a human subject in need thereof, the method includes implanting an engineered tissue construct including a plurality of spheroids in a biocompatible scaffold; wherein the spheroids include a population of hepatocytes and optionally a population of stromal cells (e.g., fibroblasts); wherein the biocompatible scaffold has an x-axis, a y-axis, and a z-axis; and wherein the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

In another aspect, the disclosure provides a kit including the engineered tissue construct of any one of the foregoing aspects, wherein the kit further includes a package insert instructing a user of the kit to implant the engineered tissue construct.

In another aspect, the disclosure provides a method of making the engineered tissue construct of any of the foregoing aspects, wherein the population of hepatocytes and the optional population of stromal cells (e.g., fibroblasts) are aggregated in spheroids; wherein the aggregated spheroids are at least partially embedded in the biocompatible scaffold; wherein the biocompatible scaffold has an x-axis, a y-axis, and a z-axis; and wherein the spheroids are allowed to non-homogenously distribute along the z-axis of the biocompatible scaffold into a layer, thereby forming the engineered tissue construct suitable for implantation in a subject. In some embodiments, the spheroids are allowed to homogenously distribute along the x-axis of the biocompatible scaffold. In some embodiments, the spheroids are allowed to homogenously distribute along the y-axis of the biocompatible scaffold.

In another aspect, the disclosure provides a method of making the engineered tissue construct of any one of the foregoing aspects, wherein the population of hepatocytes and the optional population of stromal cells (e.g., fibroblasts) are aggregated in spheroids; wherein the aggregated spheroids are at least partially embedded in the biocompatible scaffold; wherein the biocompatible scaffold has an x-axis, a y-axis, and a z-axis; wherein the spheroids are allowed to non-homogenously distribute along the z-axis of the biocompatible scaffold into a layer, thereby forming one or more of a one-sided engineered tissue construct; and wherein two or more of the one-sided engineered tissue constructs are assembled with the layers facing outwardly, thereby forming the engineered tissue construct suitable for implantation in a subject. In some embodiments, the spheroids are allowed to homogenously distribute along the x-axis of the biocompatible scaffold. In some embodiments, the spheroids are allowed to homogenously distribute along the y-axis of the biocompatible scaffold.

In some embodiments of any of the foregoing aspects, the biocompatible scaffold polymerizes in less than 3 hours (e.g., less than 2 hours, 1 hour, 59 minutes, 58 minutes, 57 minutes, 56 minutes, 55 minutes, 50 minutes, 45 minutes, 40 minutes, 30 minutes, 20 minutes, 10 minutes, or 5 minutes). In some embodiments, the biocompatible scaffold polymerizes in 45 minutes or less. In some embodiments, the biocompatible scaffold polymerizes in 30-60 minutes.

In some embodiments of the foregoing aspects, the layer is from 100 μm to 2 mm (e.g., 200 μm to 1900 μm, 300 μm to 1800 μm, 400 μm to 1700 μm, 500 μm to 1600 μm, 600 μm to 1500 μm, 700 μm to 1400 μm, 800 μm to 1300 μm, 900 μm to 1200 μm, or 1000 μm to 1100 μm) thick. For example, in some embodiments of the foregoing aspects, the layer is from 100 μm to 1 mm (e.g., 200 μm to 900 μm, 300 μm to 800 μm, 400 μm to 700 μm, or 500 μm to 600 μm) thick. In some embodiments of the foregoing aspects, the layer is from 300 μm to 500 μm.

In some embodiments of the foregoing aspects, the density of hepatocytes in the layer is from 0.06 M/cm² to 150 M/cm² (e.g., 0.07 M/cm² to 149 M/cm², 0.08 M/cm² to 148 M/cm², 0.09 M/cm² to 147 M/cm², 0.1 M/cm² to 146 M/cm², 0.2 M/cm² to 145 M/cm², 0.3 M/cm² to 140 M/cm², 0.4 M/cm² to 130 M/cm², 0.5 M/cm² to 120 M/cm², 1 M/cm² to 110 M/cm², 2 M/cm² to 100 M/cm², 3 M/cm² to 50 M/cm², 4 M/cm² to 40 M/cm², 5 M/cm² to 30 M/cm², or 10 M/cm² to 20 M/cm²).

In some embodiments of any of the foregoing aspects, the ratio of height of the biocompatible scaffold to height of the layer is from 20:1 to 1:1, e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1. In some embodiments, the ratio of height of the biocompatible scaffold to height of the layer is 2:1.

In some embodiments of any of the foregoing aspects, the biocompatible scaffold has an x-axis, a y-axis, and a z-axis. In some embodiments, the z-axis of the biocompatible scaffold is from 500 μm to 5 mm (e.g., 600 μm to 4 mm, 700 μm to 3 mm, 800 μm to 2 mm, or 900 μm to 1 mm). For example, in some embodiments, the z-axis of the biocompatible scaffold is 2 mm.

In some embodiments of any of the foregoing aspects, the population of hepatocytes includes 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes.

In some embodiments of any of the foregoing aspects, the optional population of stromal cells (e.g., fibroblasts) includes up to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², from about 3×10⁶ to about 1.8×10¹², from about 4×10⁶ to about 1.8×10¹², from about 5×10⁶ to about 1.8×10¹², from about 6×10⁶ to about 1.8×10¹², from about 7×10⁶ to about 1.8×10¹², from about 8×10⁶ to about 1.8×10¹², from about 9×10⁶ to about 1.8×10¹², from about 1×10⁷ to about 1.8×10¹², from about 2×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts).

In some embodiments of any of the foregoing aspects, the hepatocytes are primary human hepatocytes. In some embodiments, the hepatocytes are derived from stem cells (e.g., induced pluripotent stem cells).

In some embodiments of any of the foregoing aspects, the optional stromal cells are fibroblasts. In some embodiments, the fibroblasts are selected from the group consisting of normal human dermal fibroblasts and neonatal foreskin fibroblasts. For example, in some embodiments, the optional stromal cells are neonatal foreskin fibroblasts. In some embodiments, the stromal cells are normal human dermal fibroblasts.

In some embodiments of any of the foregoing aspects, the ratio of hepatocytes to stromal cells is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

In some embodiments of any of the foregoing aspects, the engineered tissue construct is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

In some embodiments of any of the foregoing aspects, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). In some embodiments, the density of hepatocytes is 0.5 M/mL to 25 M/mL (e.g., 0.6 M/mL to 24 M/mL, 0.7 M/mL to 23 M/mL, 0.8 M/mL to 22 M/mL, 0.9 M/mL to 21 M/mL, 1 M/mL to 20 M/mL, 5 M/mL to 15 M/mL, or 10 M/mL). In some embodiments, the density of hepatocytes is 1 M/mL to 12 M/mL (e.g., 2 M/mL to 11 M/mL, 3 M/mL to 10 M/mL, 4 M/mL to 9 M/mL, 5 M/mL to 8 M/mL, or 6 M/mL to 7 M/mL). In some embodiments, the density of hepatocytes is 9 M/mL.

In some embodiments of any of the foregoing aspects, the biocompatible scaffold includes fibrin. In some embodiments, the biocompatible scaffold includes heparin. In some embodiments, the heparin is a synthetic heparin mimetic.

In some embodiments of any of the foregoing aspects, the engineered tissue construct further includes a reinforcing agent. In some embodiments the reinforcing agent is fibrin, surgical mesh, alginate, collagen, poly(ethylene glycol), polyvinylidene acetate, polyvinylidene fluoride, poly(lactic-co-glycolic) acid, or poly (I-lactic acid).

In some embodiments, the engineered tissue construct has an ammonia clearance rate of at least 0.46 fmol/min/cell (e.g., at least 1 fmol/min/cell, 10 fmol/min/cell, 100 fmol/min/cell, or 150 fmol/min/cell). In some embodiments, the one or more engineered tissue constructs together have an ammonia clearance rate that is at least 200 μmol/min (e.g., at least 300 μmol/min, 400 μmol/min, 500 μmol/min, 1000 μmol/min, or 2000 μmol/min).

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the pedicled omentum. In some embodiments, the omentum is the greater omentum or the omental bursa. In some embodiments, the mesentery is the small intestinal mesentery. In some embodiments, the engineered tissue construct is implanted into the subject as a pedicled omental wrap or an omental wrap. In some embodiments, the implantation site is a site that is suitable for neovascularization.

In some embodiments, the engineered tissue construct is implanted into the subject at an implantation site that has a microvessel density of greater than about 3.6 vessels/mm² (e.g., greater than about 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

In some embodiments, the subject is a human.

In some embodiments, following implantation of the engineered tissue construct, the subject exhibits a level of serum ammonia of less than or equal to about 90 μmol/L (e.g., less than about 89 μmol/L, 88 μmol/L, 87 μmol/L, 86 μmol/L, 85 μmol/L, 84 μmol/L, 83 μmol/L, 82 μmol/L, 81 μmol/L, 80 μmol/L, 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L).

In some embodiments, following implantation of the engineered tissue construct, the subject exhibits a change in one or more parameters in a blood test relative to a reference level. In some embodiments, the blood test is a liver function test. For example, in some embodiments, the one or more parameters includes the level of gamma-glutamyl transferase, alkaline phosphatase, aspartate aminotransferase, alanine aminotransferase, or albumin.

In some embodiments, following implantation of the engineered tissue construct, the subject exhibits an improvement in a test of gallbladder ejection fraction. For example, in some embodiments, the test is a hepatobiliary iminodiacetic acid scan.

In some embodiments, following implantation of the engineered tissue construct, the subject exhibits a level of total bilirubin of less than or equal to about 1.2 mg/dL (e.g., less than or equal to about 1.2 mg/dl, 1.1 mg/dL, 1 mg/dl, 0.9 mg/dl, 0.8 mg/dl, 0.7 mg/dL, 0.6 mg/dL, 0.5 mg/dl, 0.4 mg/dl, 0.3 mg/dl, 0.2 mg/dl, or 0.1 mg/dL).

In some embodiments, following implantation of the engineered tissue construct, the subject exhibits a level of direct bilirubin of less than or equal to about 1.7 mg/dl (e.g., less than or equal to about 1.6 mg/dl, 1.5 mg/dl, 1.4 mg/dl, 1.3 mg/dl, 1.2 mg/dL, 1.1 mg/dl, 1 mg/dl, 0.9 mg/dl, 0.8 mg/dl, 0.7 mg/dl, 0.6 mg/dl, 0.5 mg/dl, 0.4 mg/dL, 0.3 mg/dl, 0.2 mg/dl, or 0.1 mg/dL).

In some embodiments, following implantation of the engineered tissue construct, the subject exhibits a level of bilirubin of less than or equal to about 1 mg/dl (0.9 mg/dl, 0.8 mg/dl, 0.7 mg/dl, 0.6 mg/dl, 0.5 mg/dl, 0.4 mg/dl, 0.3 mg/dl, 0.2 mg/dl, or 0.1 mg/dL).

In some embodiments, following implantation of the engineered tissue construct, the engineered tissue construct persists for at least two weeks (e.g., at least three weeks, one month, two months, three months, four months, five months, six months, seven months, ten months, eleven months, one year, five years, ten years, or one hundred years). For example, in some embodiments, following implantation of the engineered tissue construct, the engineered tissue construct persists for at least one month. In some embodiments, following implantation of the engineered tissue construct, the engineered tissue construct persists for at least two months.

In some embodiments, the subject in need thereof has Crigler-Najjar syndrome. In some embodiments, the Crigler-Najjar syndrome is Crigler-Najjar syndrome type I or hyperbilirubinemia (e.g., in a subject having Crigler-Najjar syndrome) type II.

In some embodiments, the subject in need thereof has a urea cycle disorder.

In some embodiments, the subject in need thereof has acute liver failure. In some embodiments, the subject may be any weight. In some embodiments, the subject weighs less than 15 kg (e.g., less than 14 kg, less than 13, kg, less than 12 kg, less than 11 kg, less than 10 kg, less than 9 kg, less than 8 kg, less than 7 kg, less than 6 kg, less than 5 kg, less than 4 kg, or less than 3 kg).

In another aspect, the disclosure provides a kit that includes an engineered tissue construct, wherein the kit further includes a package insert instructing a user of the kit to implant the engineered tissue construct into the subject in accordance with the method of any of the foregoing aspects.

The present disclosure also provides compositions and methods that can be used for treating Crigler-Najjar syndromes. Using the compositions and methods of the disclosure, a patient (e.g., a mammalian patient, such as a human patient) having Crigler-Najjar syndrome may be administered an engineered tissue construct including a population of hepatocytes and optionally a population of stromal cells in amounts effective to treat hyperbilirubinemia.

In another aspect, the disclosure provides a method of treating hyperbilirubinemia in a subject having Crigler-Najjar syndrome. the method including implanting one or more (e.g., two, three, four, or five) engineered tissue construct including a population of hepatocytes and optionally a population of stromal cells (e.g., fibroblasts) in amounts effective to treat hyperbilirubinemia in the subject.

In another aspect, the disclosure provides a method of treating Crigler-Najjar syndrome, the method including implanting one or more (e.g., two, three, four, or five) engineered tissue construct including a population of hepatocytes and optionally a population of stromal cells in amounts effective to treat Crigler-Najjar syndrome.

In some embodiments of the foregoing aspect, the Crigler-Najjar syndrome is Crigler-Najjar syndrome type I or Crigler-Najjar syndrome type II. In another aspect, the disclosure provides a method of reducing bilirubin (e.g., unconjugated bilirubin levels in a subject in need thereof (e.g., a subject having Crigler-Najjar syndrome), the method including implanting one or more (e.g., two, three, four, or five) engineered tissue construct including a population of hepatocytes and optionally a population of stromal cells (e.g., fibroblasts) in amounts effective to reduce bilirubin levels in the subject.

In some embodiments of any of the foregoing aspects, the population of hepatocytes includes an amount of hepatocytes that is equivalent to 0.5% to 30% (e.g., 0.5% to 30%, 0.6% to 30%, 0.7% to 30%, 0.8% to 30%, 0.9% to 30%, 1% to 30%, 2% to 30%, 3% to 30%, 4% to 30%, 5% to 30%, 10% to 30%, or 20% to 30%) of the total liver mass of the subject.

In some embodiments of any of the foregoing aspects, the population of hepatocytes includes an amount of hepatocytes that is equivalent to 0.5% to 20% (e.g., 0.5% to 20%, 0.6% to 20%, 0.7% to 20%, 0.8% to 20%, 0.9% to 20%, 1% to 20%, 2% to 20%, 3% to 20%, 4% to 20%, 5% to 20%, or 10% to 20%) of the mass of the liver reserve of the subject.

In some embodiments of any of the foregoing aspects, the population of hepatocytes includes 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8× 10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes.

In some embodiments of any of the foregoing aspects, the optional population of stromal cells (e.g., fibroblasts) includes 0 to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², from about 3×10⁶ to about 1.8×10¹², from about 4×10⁶ to about 1.8×10¹², from about 5×10⁶ to about 1.8×10¹², from about 6×10⁶ to about 1.8×10¹², from about 7×10⁶ to about 1.8×10¹², from about 8×10⁶ to about 1.8×10¹², from about 9×10⁶ to about 1.8×10¹², from about 1×10⁷ to about 1.8×10¹², from about 2×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts). In some embodiments, the hepatocytes are primary human hepatocytes. In some embodiments, the hepatocytes are derived from stem cells (e.g., induced pluripotent stem cells).

In some embodiments, the optional stromal cells are fibroblasts. In some embodiments, the fibroblasts are selected from the group consisting of normal human dermal fibroblasts and neonatal foreskin fibroblasts. For example, in some embodiments, the fibroblasts are neonatal foreskin fibroblasts. In some embodiments, the fibroblasts are normal human dermal fibroblasts.

In some embodiments of any of the foregoing aspects, the ratio of hepatocytes to stromal cells (e.g., fibroblasts) is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

In some embodiments of any of the foregoing aspects, the implant is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). In some embodiments, the density of hepatocytes is 3 M/mL to 12 M/mL.

In some embodiments of any of the foregoing aspects, the engineered tissue construct further includes a biocompatible hydrogel scaffold. For example, in some embodiments, the biocompatible scaffold includes fibrin. In some embodiments, the biocompatible scaffold includes heparin. In some embodiments, the heparin is a synthetic heparin mimetic.

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the omentum is the pedicled omentum. In some embodiments, the mesentery is the small intestinal mesentery. In some embodiments, the implant is implanted into the subject as a pedicled omental wrap or an omental wrap. In some embodiments, the implantation site is a site that is suitable for neovascularization.

In some embodiments, the engineered tissue construct is implanted into the subject at an implantation site that has a microvessel density of greater than about 3.6 vessels/mm² (e.g., greater than 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

In some embodiments, the engineered tissue construct provides a microenvironment that promotes the persistence of hepatocyte survival for at least three months (e.g., at least three months, at least four months, at least five months, at least six months, at least one year, at least five years, or at least ten years) in the subject.

In some embodiments, the subject is a human.

In some embodiments, following implantation of the engineered tissue construct, the subject exhibits a level of total bilirubin of less than or equal to about 1.2 mg/dl (e.g., less than or equal to 1.1 mg/dl, 1 mg/dl, 0.9 mg/dl, 0.8 mg/dl, 0.7 mg/dl, 0.6 mg/dl, 0.5 mg/dl, 0.4 mg/dL, 0.3 mg/dL, 0.2 mg/dL, or 0.1 mg/dL).

In some embodiments, following implantation of the engineered tissue construct, the subject exhibits a level of direct bilirubin of less than or equal to about 1.7 mg/dl (e.g., less than or equal to 1.6 mg/dl, 1.5 mg/dl, 1.4 mg/dl, 1.3 mg/dl, 1.2 mg/dl, 1.1 mg/dL, 1 mg/dl, 0.9 mg/dl, 0.8 mg/dl, 0.7 mg/dL, 0.6 mg/dL, 0.5 mg/dl, 0.4 mg/dl, 0.3 mg/dL, 0.2 mg/dL, or 0.1 mg/dL).

In some embodiments, following implantation of the engineered tissue construct, the subject exhibits a level of bilirubin of less than or equal to about 1 mg/dl (e.g., less than or equal to 0.9 mg/dl, 0.8 mg/dl, 0.7 mg/dl, 0.6 mg/dl, 0.5 mg/dl, 0.4 mg/dL, 0.3 mg/dL, 0.2 mg/dl, or 0.1 mg/dL).

In another aspect, the disclosure provides a kit including an engineered tissue construct, wherein the kit further includes a package insert instructing a user of the kit to implant the engineered tissue construct to the subject in accordance with the method of any one of the foregoing embodiments.

### BRIEF DESCRIPTION OF THE DRAWINGS

FIG. 1 is a schematic showing the experimental outline of a study to evaluate a novel engineered tissue construct for use as a therapy in an in vivo model of acute liver failure. Beginning on day 0, each thymidine kinase-NOD/Shi-scid/IL-2Ryru #(TK-NOG) mouse in the implanted graft group (“GCV (75 mg/kg)+graft”) was implanted with two engineered tissue constructs onto the parametrial fat pad in the intraperitoneal space of the transgenic mice, each construct including 1.41×10⁶ primary human hepatocytes and 2.82×10⁶ normal human dermal fibroblasts. Two groups, the no-toxin control (“Control(PBS)”) and the toxin-control (“GCV(75 mg/kg)”) were not subjected to surgery and received no implants. Following implantation, mice underwent a blood draw on days 5, 10, 15, 20, 29, and 34; as well as a dosing of ganciclovir (GCV) on days 26 and 31, and a liver function test performed on the blood draw from days 29 and 34. Mice were sacrificed on day 41 or during continued monitoring.

FIGS. 2A and 2B are an experimental schematic and a graph, respectively, showing the serum human albumin levels (FIG. 2B) in TK-NOG mice implanted with two engineered tissue constructs (FIG. 2A and as described in FIG. 1).

FIGS. 3A-3C are an experimental schematic and a set of graphs, respectively, showing the body weight (normalized to pre-induction) of TK-NOG mice implanted with two engineered tissue constructs, as described in FIG. 1. FIG. 3A is a schematic showing the experimental outline of TK-NOG mice implanted with two engineered tissue constructs, dosed with GCV on day 26 and 31, and tested for levels of liver enzymes on day 29 and 34. Body weight was tested daily. FIGS. 3B and 3C show graphs of body weight of TK-NOG no surgery/implantation PBS control mice (Control PBS), TK-NOG no surgery/implantation+toxin control mice (GCV), and TK-NOG mice implanted with two engineered tissue constructs (GCV+graft). The body weight of said TK-NOG mice treated with 50 mg/kg of GCV or 75 mg/kg of GCV are shown in FIGS. 3B and 3C, respectively.

FIGS. 4A and 4B are a set of graphs showing the serum levels of liver enzymes alkaline phosphatase (ALP), aspartate aminotransferase (AST), and alanine aminotransferase (ALT), and bilirubin in TK-NOG no surgery/implantation PBS control mice (PBS), TK-NOG no surgery/implantation+toxin control mice (GCV), and TK-NOG mice implanted with two engineered tissue constructs (GCV+graft), as described in FIG. 1, on day 29 (FIG. 4A) and day 34 (FIG. 4B), respectively.

FIG. 5 is a graph showing the probability of survival on days post-implantation of TK-NOG no surgery/implantation PBS control mice (Control PBS), TK-NOG no surgery/implantation+toxin control mice (GCV), and TK-NOG mice implanted with two engineered tissue constructs (GCV+graft), as described in FIG. 1, and treated with 75 mg/kg of GCV.

FIG. 6 is a photomicrograph showing the expression of CK18-positive human hepatocytes and CD31-positive mouse endothelial cells in engineered tissue constructs explanted from TK-NOG mice treated with 50 mg/kg of GCV.

FIG. 7 is a schematic showing the steps of a manufacturing build for engineered tissue constructs. In Step 1, stromal cells (e.g., fibroblasts) (e.g., normal human dermal fibroblasts (NHDF)) are thawed from their respective working cell bank (WCB), expanded, and tested to measure viability and cell count. Hepatocytes (e.g., primary human hepatocytes (PHH)) are thawed from the hepatocyte master cell bank (MCB) and tested to measure viability and cell count (Step 2); prior to being combined at a ratio (e.g., 1:2) with stromal cells (e.g., fibroblasts) (e.g., NHDF), centrifuged into arrays of pyramidal microwells, and incubated for 2-3 days to promote self-assembly of the cells into multicellular hepatic aggregates. Hepatic aggregates are deemed acceptable for encapsulation after microscopic confirmation of compaction. In Step 6, the hepatocyte/fibroblast aggregates are encapsulated with a solution (e.g., fibrinogen) that is polymerized (e.g., with thrombin). These encapsulation steps occur within a mold (e.g., a cylindrical mold) that controls the overall dimensions of the graft to be about 2 mm in thickness and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension and targets about 2 mm in thickness.

FIG. 8 is a schematic showing the experimental outline of a study to evaluate the prophylactic effect of various dosages of an engineered tissue construct in an in vivo model of acute liver failure. Beginning on day −1 (d-1), each TK-NOG mouse underwent a blood draw. On day 1 (d1) mice in group 3 were implanted with one engineered tissue construct, resulting in 0.7×10⁶ PHH/mouse (low dose “group 3”) or 5 engineered tissue constructs, resulting in 7×10⁶ PHH/mouse (high dose “group 3”). Following implantation, all mice underwent a blood draw on days 6, 11, 16, 21, 30, 35, and 42; as well as a dosing of GCV on days 27 and 32. Mice were sacrificed on day 42. Group 1 and 2 mice were not subjected to surgery and received no implants.

FIG. 9 is a set of graphs showing the serum levels of liver enzymes alkaline phosphatase (ALP), alanine aminotransferase (ALT), and aspartate aminotransferase (AST), in TK-NOG no surgery/implantation PBS control mice (TK-NOG), TK-NOG no surgery GCV control mice (TK-NOG+Toxin), and TK-NOG mice implanted with one engineered tissue construct, resulting in 0.7×10⁶ PHH/mouse (low dose) or 5 engineered tissue constructs, resulting in 7×10⁶ PHH/mouse (high dose) and dosed with GCV (TK-NOG+Toxin+Graft), as described in FIG. 8.

FIG. 10 is a graph showing the probability of survival on days post-implantation in TK-NOG no surgery/implantation PBS control mice (TK-NOG), TK-NOG no surgery GCV control mice (TK-NOG+Toxin), and TK-NOG mice implanted with high-dose implants (group 3) and dosed with GCV (TK-NOG+Toxin+Graft), as described in FIG. 8.

FIG. 11 is a graph showing the probability of survival on days post-implantation in TK-NOG no surgery/implantation PBS control mice (TK-NOG), TK-NOG no surgery GCV control mice (TK-NOG+Toxin), and TK-NOG mice implanted with low-dose implants (group 3) and dosed with GCV (TK-NOG+Toxin+Graft), as described in FIG. 8.

FIG. 12A and FIG. 12B are a set of schematics showing hepatocytes and stromal cells aggregated in spheroids in a biocompatible scaffold and distributed non-homogenously along the z-axis of the biocompatible scaffold, in a layer at the bottom. FIG. 12A is a set of photomicrographs of increasing dose densities, from left-to-right, of hepatocytes and stromal cells aggregated in spheroids in a biocompatible scaffold and distributed non-homogenously along the z-axis of the biocompatible scaffold, in a layer at the bottom. FIG. 12B is a schematic showing that the same number of aggregates (e.g., including hepatocytes) can be distributed in one layer (top left; e.g., a one-sided engineered tissue construct) or more than one layer (bottom left) in a biocompatible scaffold and that the same dose density of aggregates can be distributed across one layer (top left) or more than one layer (top right) in a biocompatible scaffold (e.g., a two-sided engineered tissue construct).

FIG. 13 is a schematic showing a two-sided engineered tissue construct. Hepatocytes and stromal cells are aggregated in spheroids in a set of two individual biocompatible scaffolds, each of which has the aggregates distributed non-homogenously along the z-axis of the biocompatible scaffold, in a layer at the bottom (step 1). In step 2, the two one-sided engineered tissue constructs are assembled into a two-sided engineered tissue construct with fibrin glue, such that each of the layers of aggregates is outward facing.

FIG. 14 is a set of photomicrographs of a two-sided engineered tissue construct, as described in FIG. 13, with human or bovine fibrin as the biocompatible scaffold, respectively. In both two-sided engineered tissue constructs, both layers of aggregates are facing outward, as described in FIG. 13.

FIG. 15 is a graph showing the serum human albumin levels in NOD-scid IL2Rgamma^{null} (NSG™) mice implanted with an engineered tissue construct including hepatocytes and stromal cells with (“Hepatic Aggregates with Endothelial Cords”) or without endothelial cell cords (“Hepatic Aggregates”).

FIG. 16 is an ultrasound-based image of the vascular volume of the implantation site in NSG™ mice implanted with two anatomically separated engineered tissue constructs. One engineered tissue construct (left circle) contained hepatocytes and stromal cells with endothelial cell cords, while the other (right circle) did not contain endothelial cell cords.

FIG. 17 is a schematic showing the experimental outline of a study to evaluate the prophylactic effect of various dosages of an engineered tissue construct in an in vivo model of acute liver failure. Beginning on day −1 (d-1), each thymidine kinase-NOD/Shi-scid/II-2Rγ^{null} (TK-NOG) mouse underwent a blood draw. On day 1 (d1) mice in group 3 were implanted with one engineered tissue construct, resulting in 0.7×10⁶ PHH/mouse (low dose “group 3”) or 5 engineered tissue constructs, resulting in 7×10⁶ PHH/mouse (high dose “group 3”). Following implantation, all mice underwent a blood draw on days 6, 11, 16, 21, 30, 35, and 42; as well as a dosing of GCV on days 27 and 32. Mice were sacrificed on day 42.

FIG. 18 is a set of graphs showing the serum levels of liver enzymes alkaline phosphatase (ALP), alanine aminotransferase (ALT), and aspartate aminotransferase (AST), in TK-NOG no surgery/implantation PBS control mice (TK-NOG), TK-NOG no surgery GCV control mice (TK-NOG+Toxin), and TK-NOG mice implanted with one engineered tissue construct, resulting in 0.7×10⁶ PHH/mouse (low dose) or 5 engineered tissue constructs, resulting in 7×10⁶ PHH/mouse (high dose) and dosed with GCV (TK-NOG+Toxin+Graft), as described in FIG. 17.

FIG. 19 is a graph showing the probability of survival on days post-implantation in TK-NOG no surgery/implantation PBS control mice (TK-NOG), TK-NOG no surgery GCV control mice (TK-NOG+Toxin), and TK-NOG mice implanted with high-dose implants and dosed with GCV (TK-NOG+Toxin+Graft), as described in FIG. 17.

FIG. 20 is a graph showing the probability of survival on days post-implantation in TK-NOG no surgery/implantation PBS control mice (TK-NOG), TK-NOG no surgery GCV control mice (TK-NOG+Toxin), and TK-NOG mice implanted with low-dose implants and dosed with GCV (TK-NOG+Toxin+Graft), as described in FIG. 17.

FIG. 21 is a graph showing the level of normalized serum ammonia in transgenic B6EiC3Sn a/A-Otc^{spf-ash}/J (sp^{fash}) unhealthy no-surgery/no-implant control mice (OTC^{spf-ash} mice) and transgenic sp^{fash} mice that received an engineered tissue construct (OTC^{spf-ash} mice+graft) that underwent immunosuppression and following implantation with an engineered tissue construct were challenged with NH4CI, which was administered intraperitoneally at week 1 and week 4.

FIG. 22 is a graph showing the level of unconjugated bilirubin in the serum of Gunn rats compared to control Wistar rats.

FIG. 23 is a schematic showing the experimental outline of a study in Gunn rats implanted with engineered tissue constructs described herein, including 1.41×10⁶ primary human hepatocytes and 2.82×10⁶ normal human dermal fibroblasts. Beginning on day −20 (d-20), homozygous Gunn rats and control Wistar rats were subjected to a pre-implantation blood draw every four or five days, followed by immunosuppression on day −7 (d-6), and implantation of eight engineered tissue constructs per rat on day 1 (d1). Blood was collected every four days (e.g., day 5 (d5), day 9 (d9), day 13 (d13), and day 45 (d45)), followed by a terminal bile/blood collection and sacrifice (Sac) on day 49 (d49).

FIG. 24 is a graph showing the level of unconjugated bilirubin and albumin, respectively, in the serum in homozygous male and female Gunn rats, respectively, on the days post-implantation of an engineered tissue construct, as described in FIG. 23. Unconjugated bilirubin levels were normalized to the respective levels in control Wistar rats.

FIG. 25 is a graph showing the level of unconjugated bilirubin over time after implantation of an engineered tissue construct, as described in FIG. 23, in the serum of Gunn rats as a percentage of the control pre-transplant level.

FIG. 26 is a graph showing the concentration of bilirubin diglucuronide (conjugated bilirubin) products in the bile of heterozygous Gunn rats implanted with an engineered tissue construct, as described in FIG. 23, as compared to control animals without an engineered tissue construct.

FIG. 27 is a set of graphs showing the serum human albumin levels in immunocompetent swine (Yorkshire) implanted in the mesentery, omentum, preperitoneal, or subcutaneously, respectively, with an engineered tissue construct including hepatocytes and stromal cells.

FIG. 28 is a photomicrograph showing the expression of hepatocytes and host blood vessels in the graft region of immunocompetent swine (Yorkshire) implanted with an engineered tissue construct including hepatocytes and stromal cells.

FIG. 29 is a graph showing seed layer height as a function of dose density of seeds produced by a vertical wheel bioreactor (VWB) with and without washing with ROTEA™ counterflow centrifugation system.

FIG. 30 is a set of images showing graft cross-sections of seed layer heights of various doses and seed types; VWB=vertical wheel bioreactor.

FIG. 31 is an image showing a hematoxylin and eosin stain of a 20M PHH/ml graft implanted in a NSG mouse at 30 days post-implantation. The arrows indicate the PHH survival zone. The scale bar=50 μm.

FIG. 32 is a set of images showing hematoxylin and eosin stains of 20M PHH/ml grafts implanted in NSG mice at 30 days post-implantation and a graph depicting the albumin produced after graft implantation in ng/ml. An10, An13, and An14 are the three animals shown in the histology, corresponding to upper left, upper right, and lower left panels, respectively. Arrows indicate the PHH survival zone. Scale bars=50 μm for the top panels and 100 μm for the bottom panel.

FIG. 33A and FIG. 33B are a set of graphs showing seed layer thickness in mm (FIG. 33A) and seed layer mass in mg (FIG. 33B) relative to the dose density in PHH M/ml.

FIG. 34 is a graph showing the level of unconjugated bilirubin in the serum of Gunn rats compared to control Wistar rats.

FIG. 35 is a schematic showing the experimental outline of a study in Gunn rats implanted with engineered tissue constructs described herein, including 1.41×10⁶ primary human hepatocytes (PHH) and 2.82×10⁶ normal human dermal fibroblasts. Beginning on day −20 (d-20), homozygous Gunn rats and control Wistar rats were subjected to a pre-implantation blood draw every four days. The Gunn rats were subjected to immunosuppression starting on day −6 (d-6), and implantation of two engineered tissue constructs per rat on day 1 (d1). The control Wistar rats did not receive immunosuppression or surgery. Blood was collected every four days (e.g., day 5 (d5), day 9 (d9), day 13 (d13), and day 45 (d45)), followed by a terminal bile/blood collection and sacrifice (Sac) on day 49 (d49).

FIG. 36 is a graph showing the level of unconjugated bilirubin and albumin, respectively, in the serum in homozygous male and female Gunn rats, respectively, on the days post-implantation of an engineered tissue construct, as described in FIG. 34. Unconjugated bilirubin levels were normalized to the respective levels in control Wistar rats.

FIG. 37 is a graph showing the level of unconjugated bilirubin over time after implantation of an engineered tissue construct, as described in FIG. 34, in the serum of Gunn rats as a percentage of the control pre-transplant level.

FIG. 38 is a graph showing the concentration of bilirubin diglucuronide (conjugated bilirubin) products in the bile of heterozygous Gunn rats implanted with an engineered tissue construct, as described in FIG. 34, as compared to control animals without an engineered tissue construct.

FIG. 39 is a schematic showing the steps of a manufacturing build for engineered tissue constructs. In Step 1, stromal cells (e.g., fibroblasts) (e.g., neonatal human dermal fibroblasts) are thawed from their respective working cell bank (WCB), expanded, and tested to measure viability and cell count. Hepatocytes (PHH) are thawed from the hepatocyte master cell bank (MCB) and tested to measure viability and cell count (Step 2); prior to being combined at a ratio (e.g., 1:2) with stromal cells (e.g., fibroblasts) (e.g., neonatal human fibroblasts), centrifuged into arrays of microwells (e.g., pyramidal microwells), and incubated for 2-3 days to promote self-assembly of the cells into multicellular hepatic aggregates. Hepatic aggregates are deemed acceptable for encapsulation after microscopic confirmation of compaction. In Step 6, the hepatocyte/fibroblast aggregates are encapsulated with a solution (e.g., a solution containing fibrinogen) that is polymerized (e.g., with thrombin). These encapsulation steps occur within a mold (e.g., a cylindrical mold) that controls the overall dimensions of the graft to be about 2 mm in thickness and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension.

### DEFINITIONS

As used herein, the terms “implanting,” “implantation,” and the like, refer to directly giving a subject (e.g., a human subject) one or more engineered tissue construct at any effective implantation site, such as a site that is suitable for neovascularization. Exemplary implantation sites include the peritoneum (e.g., retroperitoneum), peritoneal cavity (e.g., omentum or mesentery), rectus abdominis muscle, abdominal oblique muscle, quadriceps femoris muscle, extraperitoneal fat, renal capsule, an extraperitoneal site, a site on the surface of the liver, and an extrapleural site, among others.

As used herein, the term a “subject” is a mammal. Mammals include, but are not limited to, domesticated animals (e.g., cows, sheep, cats, dogs, and horses), primates (e.g., humans and non-human primates such as monkeys), rabbits, deer, and rodents (e.g., mice and rats). In certain embodiments, the subject is a human.

The term “acute liver failure” includes, but is not limited to, the conditions referred to by the terms hyperacute liver failure, acute liver failure, subacute liver failure, and fulminant hepatic failure (FHF). As used herein, fulminant hepatic failure” or “FHF” are used interchangeable and are defined as the severe impairment of hepatic functions in the absence of pre-existing liver disease. For example, FHF may result from exposure of a susceptible individual to an agent capable of producing serious hepatic injury. Examples of such agents include infectious agents, excessive alcohol, hepatotoxic metabolites, and hepatotoxic compounds (e.g., drugs). Other causes of FHF include congenital abnormalities, autoimmune disease, and metabolic disease. In many cases the precise etiology of FHF is unknown (e.g., idiopathic).

As used herein, the term “urea cycle disorder” refers to any disorder that is caused by a defect or malfunction in the urea cycle. The urea cycle is a cycle of biochemical reactions that produces urea from ammonia, a product of protein catabolismspecific types of urea cycle disorder include, but are not limited to, phosphate synthetase 1 (CPS1) deficiency, ornithine transcarbamylase (OTC) deficiency, argininosuccinate synthetase (ASS1) deficiency, argininosuccinate lyase (ASL) deficiency, arginase-1 (ARG1) deficiency, N-acetylglutamate synthetase (NAGS) deficiency, ornithine translocase (ORNT1) deficiency, and citrin deficiency. A urea cycle disorder may be characterized by an aberrant level of ammonia (e.g., an ammonia level of greater than or equal to about 80 μmol/L).

As used herein, “Crigler-Najjar syndrome” refers to a condition characterized by high levels of bilirubin in the blood (hyperbilirubinemia). Bilirubin is produced when red blood cells are broken down. This substance is removed from the body only after it undergoes a chemical reaction in the liver, which converts the toxic form of bilirubin (called unconjugated bilirubin) to a nontoxic form called conjugated bilirubin. Subjects with Crigler-Najjar syndrome have a buildup of unconjugated bilirubin in their blood (unconjugated hyperbilirubinemia). Crigler-Najjar syndrome is classified into two subtypes, type I and type II. As used herein, “Crigler-Najjar type I” refers to a subtype of Crigler-Najjar syndrome in which mutations in the B-UGT1 gene cause the resulting expressed enzyme, B-UGT, to be completely inactive. Thus, Crigler-Najjar type I patients exhibit a complete absence of B-UGT activity. As used herein, “Crigler-Najjar type II” refers to a subtype of Crigler-Najjar syndrome in which mutations in the B-UGT1 gene cause B-UGT to be partially inactive. Thus, B-UGT activity is reduced in patients with Crigler-Najjar type II and such patients exhibit a strongly reduced bilirubin conjugation capacity compared to healthy subjects.

As used herein, the term “hyperbilirubinemia” refers to a condition in which there is a higher-than-normal level of bilirubin in the blood. As used herein, the term “bilirubin” refers to a compound that occurs in the normal catabolic pathway that breaks down heme in vertebrates. This catabolismis a necessary process in the body's clearance of waste products that arise from the destruction of aged or abnormal red blood cells. As used herein, a “bilirubin test” refers to a measurement of the amount of bilirubin in a patient's blood. As used herein, the terms “comprise,” “comprising,” “includes,” and “comprised of” are synonymous with “include,” “including,” “includes” or “contain,” “containing,” “contains” and are inclusive or open-ended terms that specifies the presence of what follows e.g., a component and do not exclude or preclude the presence of additional, non-recited components, features, element, members, steps, known in the art or disclosed therein.

Whereas the terms “one or more” or “at least one,” such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the terms “amounts effect,” “effective amount,” “therapeutically effective amount,” and the like, when used in reference to an engineered tissue construct described herein, refer to a quantity of hepatocytes and stromal cells (e.g., fibroblasts) in the engineered tissue construct sufficient to, when implanted into the subject (e.g., a mammal e.g., a human) effect beneficial or desired results, such as clinical results. For example, in the context of treating acute liver failure or hyperbilirubinemia, such as in a patient having Crigler-Najjar syndrome, these terms refer to an amount of the hepatocyes and stromal cells (e.g., fibroblasts) sufficient to achieve a treatment response as compared to the response obtained without implantation of the engineered tissue construct of interest. An “effective amount,” “therapeutically effective amount,” and the like, of a engineered tissue construct of the present disclosure, also include an amount that results in a beneficial or desired result in a subject as compared to a control.

As used herein, the terms “treat” and “treatment” refer to therapeutic treatment, in which the object is to prevent or slow down (lessen) an undesired physiological change, such as the progression of acute liver failure, Crigler-Najjar syndrome, a urea cycle disorder, or hyperbilirubinemia (e.g., in a subject having Crigler-Najjar syndrome). Beneficial or desired clinical results include, but are not limited to, the reduction of ammonia, an improvement in a test of gallbladder ejection fraction, the alleviation of ALF symptoms, or alleviation of hyperbilirubinemia symptoms. The concentration of ammonia protein or the gallbladder ejection fraction may be determined using assays known in the art, for example, using a hepatobiliary iminodiacetic acid scan.

As used in the context of the present disclosure, an “engineered tissue construct” refers to a mixture of cultured hepatocytes (e.g., primary human hepatocytes) and stromal cells (e.g., fibroblasts) (e.g., neonatal foreskin stromal cells (e.g., fibroblasts)). The relative volume of the engineered tissue construct may be between 0.1 mL to 5 L, .g., 0.5 mL to 5 L. In some embodiments, the engineered tissue construct further includes a biocompatible hydrogel scaffold (e.g., fibrin).

Cells can be from established cell lines or they can be primary cells, where “primary cells,” “primary cell lines,” and “primary cultures” are used interchangeably herein to refer to cells and cells cultures that have been derived from and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, etc. are most conveniently harvested by biopsy. An appropriate solution can be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry and Friend, 1969, J. Cell Biol. 43:506-520) which can be adapted for human liver biopsy or autopsy material (e.g., to garner primary human hepatocytes).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a hydrogel scaffold is considered “biocompatible” when is it does not exhibit toxicity when introduced into a subject (e.g., a human). In the context of the present disclosure, it is preferable that the biocompatible hydrogel scaffold does not exhibit toxicity towards the cells of the engineered tissue construct or when implanted in vivo in a subject (e.g., a human). Hepatotoxicity can be measured, for example, by determining hepatocytes apoptotic death rate (e.g., wherein an increase in apoptosis is indicative of hepatotoxicity), transaminase levels (e.g., wherein an increase in transaminase levels is indicative of hepatotoxicity), ballooning of the hepatocytes (e.g., wherein an increase in ballooning is indicative of hepatotoxicity), microvesicular steatosis in the hepatocytes (e.g., wherein an increase in steatosis is indicative of hepatotoxicity), biliary cells death rate (e.g., wherein an increase in biliary cells death rate is indicative of hepatotoxicity), γ-glutamyl transpeptidase (GGT) levels (e.g., wherein an increase in GGT levels is indicative of hepatotoxicity). A biocompatible hydrogel scaffold can include, but is not limited to, fibrin and heparin.

As used herein, the term “hydrogel” refers to a network of polymer chains that are hydrophilic in nature, such that the material absorbs a high volume of water or other aqueous solution. Hydrogels can include, for example, at least 70% v/v water, at least 80% v/v water, at least 90% v/v water, at least 95%, 96%, 97%, 98% and even 99% or greater v/v water (or other aqueous solution). Hydrogels can include natural or synthetic polymers, the polymeric network often featuring a high degree of crosslinking. Hydrogels also possess a degree of flexibility very similar to natural tissue, due to their significant water content. Hydrogels are particularly useful in tissue engineering applications as scaffolds for culturing cells. In certain embodiments, the hydrogels are made of biocompatible polymers.

The term “adherence material” is a material incorporated into the cell mixture disclosed herein to which a cell or microorganism has some affinity, such as a binding agent. The material can be incorporated, for example, into a hydrogel, e.g., prior to implantation of the engineered cell mixture. The material and a cell or microorganism interact through any means including, for example, electrostatic or hydrophobic interactions, covalent binding, or ionic attachment. The material may include, but is not limited to, antibodies, proteins, peptides, nucleic acids, peptide aptamers, nucleic acid aptamers, sugars, proteoglycans, or cellular receptors.

As used herein, the term “decompose,” refers to the physiological process of biochemical degradation, digestion, and/or break down of a molecule of interest (e.g., ammonia), to remove the molecule from the body (e.g., by renal clearance).

As used herein, the term “suitable for neovascularization” refers to conditions and/or environmental characteristics fit for the formation of new blood vessels. Generally, neovascularization means the formation of new blood vessels in injured tissue or in tissue not normally containing blood vessels or the formation of novel blood vessels (e.g., arterioles, venules, and capillaries) of a higher density than usual in said tissue. For example, a site that is suitable for neovascularization may have an existing microvessel density of greater than about 3.6 vessels/mm² (e.g., greater than about 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

As used herein, the terms “liver function test” and “LFT” refer to a hepatic panel (e.g., a group of blood tests that provide information about the state of a patient's liver). A hepatic panel may include measurement of the level of gamma-glutamyl transferase, the level of alkaline phosphatase, the level of aspartate aminotransferase, the level of alanine aminotransferase, the level of albumin, the level of bilirubin, the prothrombin time, the activated partial thromboplastin time, or a combination thereof.

As used herein, a “bilirubin test” refers to a measurement of the amount of bilirubin in a patient's (e.g., a human patient) blood.

As used herein, the term “age-adjusted norms” refers to the process of a normalization of data by age, which is a technique that is used to allow populations of subjects to be compared when the age profiles of the populations are different. As used herein, the term “norm” refers to data that does not undergo a normalization by age, as populations of subjects across age profiles are similar.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an 15 increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

As used herein, the terms “seed layer thickness” and “seed layer height” refer to the height of the engineered tissue construct determined prior to implantation.

By a “reference” is meant any useful reference used to compare protein levels related to hyperbilirubinemia, or one or more symptoms thereof. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having Crigler-Najjar syndrome, or one or more symptoms thereof; a sample from a subject that is diagnosed with Crigler-Najjar syndrome, or one or more symptoms thereof; a sample from a subject that has been treated for hyperbilirubinemia, or one or more symptoms thereof; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having hyperbilirubinemia, or one or more symptoms thereof. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

### DETAILED DESCRIPTION

The present disclosure provides compositions and methods that can be used for treating acute liver failure (ALF). In accordance with the compositions and methods described herein, a subject (e.g., a human) having ALF may be implanted with one or more engineered tissue construct that include a population of hepatocytes and an optional population of stromal cells (e.g., fibroblasts).

The present disclosure also provides compositions and methods that can be used for treating hyperbilirubinemia (e.g., in a subject having Crigler-Najjar syndrome) or reducing bilirubin levels in a subject in need thereof. In accordance with the compositions and methods described herein, a subject (e.g., a human) having Crigler-Najjar syndrome may be implanted with one or more engineered tissue construct that includes a population of hepatocytes and an optional population of stromal cells (e.g., fibroblasts).

The present disclosure is based, at least in part, on the discovery that engineered tissue constructs, including hepatocytes and optionally stromal cells (e.g., fibroblasts), can be implanted, and used for methods of treating ALF. Specifically, the successful treatment of ALF requires the ongoing survival of hepatocytes (e.g., implanted hepatocytes), including a persistence of hepatocyte survival that extends beyond at least three months' time. The engineered tissue constructs described herein provide a microenvironment that promotes hepatocyte survival for at least three months (e.g., at least three months, at least four months, at least five months, at least six months, or at least one year), thereby addressing the outstanding significant unmet characteristics that are associated with the therapies that currently exist for the treatment of ALF.

The present disclosure is also based, at least in part, on the discovery that engineered tissue constructs, including hepatocytes and optionally stromal cells (e.g., fibroblasts), can be implanted and used for methods of treating hyperbilirubinemia, thereby addressing the outstanding significant unmet medical need associated with the symptoms of hyperbilirubinemia that exist in patients with Crigler-Najjar syndromes. The disclosure is also based, at least in part, on the inventors surprising discovery that implantation of engineered tissue constructs significantly increases the level of conjugated bilirubin products.

The present disclosure also provides engineered tissue constructs that include a population of hepatocytes, an optional population of stromal cells, and a biocompatible scaffold and methods of making the same. These compositions can be used, e.g., for treating acute liver failure, a urea cycle disorder, or hyperbilirubinemia (e.g., in a subject having Crigler-Najjar syndrome) in a subject (e.g., a human subject) in need thereof.

To date, most cell-based pre-vascularization strategies of engineered tissue constructs have utilized randomly seeded cells embedded within a three-dimensional matrix. For example, it has been shown that the speed of vascularization can be increased by allowing endothelial cells to form rudimentary networks in vitro prior to implantation. Furthermore, it has been demonstrated that implantation of scaffolds pre-seeded with endothelial cells can facilitate tubulogenesis (the formation of interconnected web-like networks of interconnected endothelial cells) within the scaffold and eventual anastomosis (connection) of the newly formed tubules to host vessels within days to weeks. In contrast, the present disclosure is based, at least in part, on the discovery that engineered tissue constructs, including hepatocytes and stromal cells that together include at least 70% of the cells in the engineered tissue construct (with negligible endothelial cells e.g., no greater than 30%), can achieve superior functional outcomes, such as significantly enhanced albumin production, lower levels of ammonia, improved survival, elevated levels of liver enzymes, all demonstrative of engineered tissue construct integration and survival and enhanced vascularization. The engineered tissue constructs described herein can be used for methods of treating liver dysfunction (e.g., acute liver failure, a urea cycle disorder, or hyperbilirubinemia (e.g., in a subject having Crigler-Najjar syndrome)).

The sections that follow provide a description of engineered tissue construct. The following sections also describe various implantation sites and parameters for clinical monitoring following implantation of the engineered tissue construct that may be used in conjunction with the compositions and methods of the disclosure.

Acute Liver Failure ALF is the appearance of severe complications after the first signs of liver disease (e.g., jaundice) in a patient (e.g., a human patient). ALF includes a number of conditions, most of which involve severe hepatocyte injury or necrosis. In most cases of ALF, massive necrosis of hepatocytes occurs. Altered mental status (hepatic encephalopathy) and coagulopathy in the setting of a hepatic disease generally define ALF. Consequently, ALF is generally clinically defined as the development of coagulopathy, usually an international normalized ratio (a measure of the time it takes blood to clot compared to an average value-INR) of greater than 1.5 (e.g., 2, 2.5, 3, 4, or 5), and any degree of mental alteration (encephalopathy) in a patient without preexisting cirrhosis and with an illness of less than 26 weeks (e.g., less than 25 weeks, less than 24, weeks, less than 23 weeks, less than 22 weeks, less than 21 weeks, and less than 20 weeks) duration. ALF indicates that the liver has sustained severe damage resulting in the dysfunction of about 80-90% of liver cells.

ALF occurs when the liver fails rapidly. Hyperacute liver failure is characterized as failure of the liver within one week. ALF is characterized as the failure of the liver within 8-28 days. Subacute liver failure is characterized as the failure of the liver within 4-12 weeks.

In some embodiments, the human subject having ALF weighs less than 15 kg (e.g., less than 15 kg, less than 14 kg, less than 13, kg, less than 12 kg, less than 11 kg, less than 10 kg, less than 9 kg, less than 8 kg, less than 7 kg, less than 6 kg, less than 5 kg, less than 4 kg, or less than 3 kg). For example, in some embodiments, the human subject weighs less than 14 kg. In some embodiments, the human subject weighs less than 13 kg. In some embodiments, the human subject weighs less than 12 kg. In some embodiments, the human subject weighs less than 11 kg. In some embodiments, the human subject weighs less than 10 kg. In some embodiments, the human subject weighs less than 9 kg. In some embodiments, the human subject weighs less than 8 kg. In some embodiments, the human subject weighs less than 7 kg. In some embodiments, the human subject weighs less than 6 kg. In some embodiments, the human subject weighs less than 5 kg. In some embodiments, the human subject weighs less than 4 kg. In some embodiments, the human subject weighs less than 3 kg.

In some embodiments, the human subject having ALF weighs about 15 kg. In some embodiments, the human subject weighs about 14 kg. In some embodiments, the human subject weighs about 13 kg. In some embodiments, the human subject weighs about 12 kg. In some embodiments, the human subject weighs about 11 kg. In some embodiments, the human subject weighs about 10 kg. In some embodiments, the human subject weighs about 9 kg. In some embodiments, the human subject weighs about 8 kg. In some embodiments, the human subject weighs about 7 kg. In some embodiments, the human subject weighs about 6 kg. In some embodiments, the human subject weighs about 5 kg. In some embodiments, the human subject weighs about 4 kg. In some embodiments, the human subject weighs about 3 kg. In some embodiments, the human subject (e.g., having ALF) may be any weight.

In some embodiments, the method includes treating a subject having ALF, the method including implanting an engineered tissue construct including a population of hepatocytes and a population of stromal cells.

### Crigler-Najjar Syndrome

Hyperbilirubinemia is a condition in which there is an accumulation of bilirubin in the blood and serum bile acids appear to remain normal. A subpopulation of subjects experiencing hyperbilirubinemia have Crigler-Najjar syndrome (e.g., Crigler-Najjar syndrome type I and Crigler-Najjar syndrome type II). Subjects with Crigler-Najjar syndrome type I have total serum bilirubin levels exceeding 20 mg/dL. Their bile is almost completely composed of unconjugated bilirubin, with low levels of mono-glucuronide bilirubin. Subjects with Crigler-Najjar syndrome type II have total serum bilirubin levels in the range of 3.5-20 mg/dl. The disclosure provides a method of treating hyperbilirubinemia. In some embodiments, the method includes treating hyperbilirubinemia in a subject having Crigler-Najjar syndrome, the method including implanting one or more engineered tissue construct including a population of hepatocytes and a population of stromal cells in amounts effective to treat hyperbilirubinemia in the subject. In some embodiments, the Crigler-Najjar syndrome is Crigler-Najjar syndrome type I. In some embodiments, the Crigler-Najjar syndrome is Crigler-Najjar syndrome type II.

In some embodiments, the method includes treating Crigler-Najjar syndrome in a subject in need thereof, the method including implanting one or more engineered tissue construct including a population of hepatocytes and a population of stromal cells in an effective amount. In some embodiments, the Crigler-Najjar syndrome is Crigler-Najjar syndrome type I. In some embodiments, the Crigler-Najjar syndrome is Crigler-Najjar syndrome type II.

In some embodiments, the method includes treating a subject having hyperbilirubinemia, the method including implanting an engineered tissue construct including a population of hepatocytes and a population of stromal cells.

In some embodiments, the method includes treating a subject having Crigler-Najjar syndrome, the method including implanting an engineered tissue construct including a population of hepatocytes and a population of stromal cells.

In some embodiments, the Crigler-Najjar syndrome is Crigler-Najjar syndrome type I. In some embodiments, the Crigler-Najjar syndrome is Crigler-Najjar syndrome type II.

### Urea Cycle Disorders

The urea cycle is a cycle of biochemical reactions that produces urea from ammonia, a product of protein catabolismthe urea cycle includes five key enzymes including carbamoyl phosphate synthetase 1 (CPS1), ornithine transcarbamoylase (OTC), argininosuccinate synthetase (ASS1), argininosuccinate lyase (ASL), and arginase 1 (ARG1), but also requires other enzymes, such as N-acetylglutamate synthetase (NAGS), and mitochondrial amino acid transporters, such as ornithine translocase (ORNT1) and citrin. The urea cycle mainly occurs in the mitochondria of liver cells. The urea produced by the liver enters the bloodstream where it travels to the kidneys and is ultimately excreted in urine. Genetic defects in any of the enzymes or transporters in the urea cycle can cause a urea cycle or a symptom thereof.

In some embodiments, the method includes treating a subject having a urea cycle disorder, the method including implanting an engineered tissue construct including a population of hepatocytes and a population of stromal cells.

### Engineered Tissue Constructs

The engineered tissue constructs described herein includes a population of hepatocytes and optionally a population of stromal cells (e.g., fibroblasts).

In some embodiments, the engineered tissue construct provides a microenvironment that promotes the persistence of hepatocyte survival for least three months (e.g., at least three months, at least four months, at least five months, at least six months, at least one year, at least five years, at least ten years, or the lifetime of a patient in which the engineered tissue construct is implanted into) in the subject. For example, in some embodiments, the engineered tissue construct provides a microenvironment that promotes the persistence of hepatocyte survival for least four months. In some embodiments, the engineered tissue construct provides a microenvironment that promotes the persistence of hepatocyte survival for least five months. In some embodiments, the engineered tissue construct provides a microenvironment that promotes the persistence of hepatocyte survival for least six months. In some embodiments, the engineered tissue construct provides a microenvironment that promotes the persistence of hepatocyte survival for least one year. In some embodiments, the engineered tissue construct provides a microenvironment that promotes the persistence of hepatocyte survival for least five years. In some embodiments, the engineered tissue construct provides a microenvironment that promotes the persistence of hepatocyte survival for least ten years.

In another aspect, the cellular compositions are provided in the form of an aggregate of the hepatocyte and optional stromal cell populations. In some embodiments, the hepatocyte and optional stromal cell populations are admixed under conditions which cause the two cell populations to form aggregates. In some embodiments, the hepatocyte and optional stromal cell populations are admixed using tissue fabrication techniques. In some embodiments, the hepatocyte and optional stromal cell populations are co-cultured. In some embodiments, the hepatocyte and optional stromal cell populations are admixed using tissue fabrication techniques. In some embodiments, the hepatocyte and optional stromal cell populations are co-cultured. In some embodiments, the hepatocyte and optional stromal cell populations are cocultured by hanging drop, microwell molding, non-adhesive surfaces, spheroid suspension culture using a spinner flask, vertical wheel bioreactor, horizontal wheel bioreactor, or a microfluidic spheroid system. Additional methods include those using acoustical waves and using positively charged surfaces on a plate.

In other aspects, the compositions provided herein can contain additional components, including but not limited to, growth factors, ligands, cytokines, drugs, etc. In some embodiments, the cell mixtures can include molecules which elicit additional microenvironmental cues such as small molecules or growth factors which stimulate or enhance proliferation and expansion of a cell population.

The properties of the cell aggregates of the present disclosure can be varied to suit a particular application. In certain embodiments, the density of the cell aggregates can be changed. In certain embodiments, cell aggregates of different diameters can be fabricated. In certain embodiments, the overall network organization of the one or more cell aggregates can be defined, for example, by the number, three-dimensional organization, alignment, diameters, density, and the like.

In certain embodiments, the engineered cell composition or tissue construct can contain one or more bioactive substances. Examples of bioactive substance(s) include, but are not limited to, hormones, neurotransmitters, growth factors, hormone, neurotransmitter or growth factor receptors, interferons, interleukins, chemokines, cytokines, colony stimulating factors, chemotactic factors, extracellular matrix components, and adhesion molecules, ligands and peptides; such as growth hormone, parathyroid hormone bone morphogenetic protein, transforming growth factor-alpha, TGF-beta1, TGF-beta2, stromal cell growth factor (e.g., fibroblast growth factor), granulocyte/macrophage colony stimulating factor, epidermal growth factor, platelet derived growth factor, insulin-like growth factor, scatter factor/hepatocyte growth factor, fibrin, dextran, matrix metalloproteinases, collagen, fibronectin, vitronectin, hyaluronic acid, an RGD-containing peptide or polypeptide, an angiopoietin and vascular endothelial cell growth factor.

In certain embodiments, the engineered cell mixtures or tissue constructs disclosed herein include one or more adherence materials to facilitate maintenance of the desired phenotype of the grafted cells in vivo. The material may include, but is not limited to, antibodies, proteins, peptides, nucleic acids, peptide aptamers, nucleic acid aptamers, sugars, proteoglycans, or cellular receptors.

The type of adherence materials (e.g., extra-cellular matrix (ECM) materials, sugars, proteoglycans etc.) will be determined, in part, by the cell type or types (e.g., hepatocytes and stromal cells (e.g., fibroblasts)) to be cultured. ECM molecules found in a cell's native microenvironment are useful in maintaining the function of both primary cells, precursor cells, and/or cell lines.

In some embodiments, the engineered tissue constructs further includes a biocompatible scaffold (e.g., biocompatible hydrogel scaffold). For example, in some embodiments, the biocompatible scaffold is fibrin. In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel that includes the synthetic polymer described herein can bind various bioactive agents, e.g., growth factors, thereby preventing the bioactive agents from diffusing away and maintaining the bioactive agents at a high concentration locally, so that they can act on cells and promote various cell functions.

In some embodiments, the implant or engineered tissue construct is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume. For example, in some embodiments, the implant or engineered tissue construct is from 0.2 mL to 5 L in volume. In some embodiments, the implant or engineered tissue construct is from 0.3 mL to 5 L in volume. In some embodiments, the implant or engineered tissue construct is from 0.4 mL to 5 L in volume. In some embodiments, the implant or engineered tissue construct is from 0.5 mL to 5 L in volume. In some embodiments, the implant or engineered tissue construct is from 1 mL to 5 L in volume. In some embodiments, the implant or engineered tissue construct is from 5 mL to 5 L in volume. In some embodiments, the implant or engineered tissue construct is from 10 mL to 5 L in volume. In some embodiments, the implant or engineered tissue construct is from 100 mL to 5 L in volume. In some embodiments, the implant or engineered tissue construct is from 1 ml to 5 L in volume. In some embodiments, the implant or engineered tissue construct is from 2 mL to 5 L in volume. In some embodiments, the implant or engineered tissue construct is from 3 mL to 5 L in volume. In some embodiments, the implant or engineered tissue construct is from 4 mL to 5 L in volume.

In some embodiments, the engineered tissue construct further includes a reinforcing agent. In some embodiments the reinforcing agent is selected from the list including fibrin, surgical mesh, alginate, collagen, poly(ethylene glycol), polyvinylidene acetate (PVDA), polyvinylidene fluoride (PVDF), poly(lactic-co-glycolic) acid (PLGA), and poly (I-lactic acid) (PLLA). In some embodiments the reinforcing agent is fibrin. In some embodiments the reinforcing agent is surgical mesh. In some embodiments the reinforcing agent is alginate. In some embodiments the reinforcing agent is collagen. In some embodiments the reinforcing agent is poly(ethylene glycol). In some embodiments the reinforcing agent is PVDA. In some embodiments the reinforcing agent is PVDF. In some embodiments the reinforcing agent is PLGA. In some embodiments the reinforcing agent is PLLA. In some embodiments the reinforcing agent is any suitable agent.

In some embodiments, the engineered tissue construct has an ammonia clearance rate of at least 0.46 fmol/min/cell (e.g., at least 0.47 fmol/min/cell, 0.48 fmol/min/cell, 0.49 fmol/min/cell, 0.5 fmol/min/cell, 1 fmol/min/cell, 10 fmol/min/cell, 100 fmol/min/cell, or 150 fmol/min/cell). For example, in some embodiments, the engineered tissue construct has an ammonia clearance rate of at least 0.47 fmol/min/cell. In some embodiments, the engineered tissue construct has an ammonia clearance rate of at least 0.48 fmol/min/cell. In some embodiments, the engineered tissue construct has an ammonia clearance rate of at least 0.49 fmol/min/cell. In some embodiments, the engineered tissue construct has an ammonia clearance rate of at least 0.5 fmol/min/cell. In some embodiments, the engineered tissue construct has an ammonia clearance rate of at least 1 fmol/min/cell. In some embodiments, the engineered tissue construct has an ammonia clearance rate of at least 10 fmol/min/cell. In some embodiments, the engineered tissue construct has an ammonia clearance rate of at least 100 fmol/min/cell. In some embodiments, the engineered tissue construct has an ammonia clearance rate of at least 150 fmol/min/cell.

In some embodiments, the engineered tissue construct has an ammonia clearance rate of about 0.46 fmol/min/cell. In some embodiments, the engineered tissue construct has an ammonia clearance rate of 1 fmol/min/cell. In some embodiments, the engineered tissue construct has an ammonia clearance rate of about 10 fmol/min/cell. In some embodiments, the engineered tissue construct has an ammonia clearance rate of about 100 fmol/min/cell. In some embodiments, the engineered tissue construct has an ammonia clearance rate of about 150 fmol/min/cell. In some embodiments, the one or more engineered tissue constructs together have an ammonia clearance rate that is at least 200 μmol/min (e.g., at least 300 μmol/min, 400 μmol/min, 500 μmol/min, 1000 μmol/min, or 2000 μmol/min). For example, in some embodiments, the one or more engineered tissue constructs together have an ammonia clearance rate that is at least 300 μmol/min. In some embodiments, the one or more engineered tissue constructs together have an ammonia clearance rate that is at least 400 μmol/min. In some embodiments, the one or more engineered tissue constructs together have an ammonia clearance rate that is at least 500 μmol/min. In some embodiments, the one or more engineered tissue constructs together have an ammonia clearance rate that is at least 1000 μmol/min. In some embodiments, the one or more engineered tissue constructs together have an ammonia clearance rate that is at least 2000 μmol/min.

In some embodiments, the one or more engineered tissue constructs together have an ammonia clearance rate that is about 200 μmol/min. In some embodiments, the one or more engineered tissue constructs together have an ammonia clearance rate that is about 300 μmol/min. In some embodiments, the one or more engineered tissue constructs together have an ammonia clearance rate that is about 400 μmol/min. In some embodiments, the one or more engineered tissue constructs together have an ammonia clearance rate that is about 500 μmol/min. In some embodiments, the one or more engineered tissue constructs together have an ammonia clearance rate that is about 1000 μmol/min. In some embodiments, the one or more engineered tissue constructs together have an ammonia clearance rate that is about 2000 μmol/min.

In some embodiments, the engineered tissue construct may be any shape (e.g., cylindrical, square, or square with rounded corners).

In some embodiments, the engineered tissue construct has a serpentine topography (e.g., to increase surface area).

### Persistence

In some embodiments, the engineered tissue construct provides a microenvironment that promotes the persistence of hepatocyte survival for least three months (e.g., at least three months, at least four months, at least five months, at least six months, at least one year, at least five years, or at least ten years) in the subject. For example, in some embodiments, the engineered tissue construct provides a microenvironment that promotes the persistence of hepatocyte survival for least four months. In some embodiments, the engineered tissue construct provides a microenvironment that promotes the persistence of hepatocyte survival for least five months. In some embodiments, the engineered tissue construct provides a microenvironment that promotes the persistence of hepatocyte survival for least six months. In some embodiments, the engineered tissue construct provides a microenvironment that promotes the persistence of hepatocyte survival for least one year. In some embodiments, the engineered tissue construct provides a microenvironment that promotes the persistence of hepatocyte survival for least five years. In some embodiments, the engineered tissue construct provides a microenvironment that promotes the persistence of hepatocyte survival for least ten years.

### Cell Populations

Cell populations may be optimized to maintain the appropriate morphology, phenotype, and cellular function conducive to use in the methods of the disclosure. For example, primary human hepatocytes or neonatal foreskin stromal cells (e.g., fibroblasts) can be isolated and/or pre-cultured under conditions optimized to ensure that the respective cells of choice initially have the desired morphology, phenotype and cellular function and, thus, are poised to maintain said morphology, phenotype and/or function in vivo following implantation of the engineered tissue constructs described herein.

### Hepatocytes

The engineered tissue constructs described herein include hepatocytes. In some embodiments, the hepatocytes are primary human hepatocytes (PHH). In some embodiments, the hepatocytes are derived from stem cells (e.g., induced pluripotent stem cells).

In some embodiments, the hepatocytes described herein are obtained by methods including culturing and passaging the PHH to obtain a population of expanded PHH or obtaining the population of expanded PHH from a single PHH cell. In some embodiments, obtaining the population of expanded PHH includes culturing and passaging the PHH in an appropriate cell medium for human cells (e.g., for 3-120 days (e.g., 4-119 days, 5-118 days, 10-117 days, 15-116 days, 20-115 days, 30-100 days, 40-90 days, 50-80 days, 60-70 days, or 65 days)). See e.g., U.S. Provisional Patent Application No. 63/271,441, incorporated herein by reference.

In some embodiments the engineered tissue construct includes a population of hepatocytes in an amount that is effective to treat ALF in a subject (e.g., a human).

In some embodiments, the engineered tissue construct includes a population of hepatocytes in an amount that is effective to treat hyperbilirubinemia in a subject (e.g., a human).

In some embodiments, the engineered tissue construct includes a population of hepatocytes in an amount that is effective to reduce bilirubin levels in the subject (e.g., a human).

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). For example, in some embodiments, the density of hepatocytes is 0.2 M/mL to 149 M/mL. In some embodiments, the density of hepatocytes is 0.3 M/mL to 148 M/mL. In some embodiments, the density of hepatocytes is 0.4 M/mL to 147 M/mL. In some embodiments, the density of hepatocytes is 0.5 M/mL to 146 M/mL. In some embodiments, the density of hepatocytes is 1 M/mL to 145 M/mL. In some embodiments, the density of hepatocytes is 5 M/mL to 140 M/mL. In some embodiments, the density of hepatocytes is 10 M/mL to 100 M/mL. In some embodiments, the density of hepatocytes is 20 M/mL to 50 M/mL. In some embodiments, the density of hepatocytes is 30 M/mL to 40 M/mL.

In some embodiments, the density of hepatocytes is 0.5 M/mL to 25 M/mL (e.g., 0.6 M/mL to 24 M/mL, 0.7 M/mL to 23 M/mL, 0.8 M/mL to 22 M/mL, 0.9 M/mL to 21 M/mL, 1 M/mL to 20 M/mL, 5 M/mL to 15 M/mL, or 10 M/mL). For example, in some embodiments, the density of hepatocytes is 0.6 M/mL to 24 M/mL. In some embodiments, the density of hepatocytes is 0.7 M/mL to 23 M/mL. In some embodiments, the density of hepatocytes is 0.8 M/mL to 22 M/mL. In some embodiments, the density of hepatocytes is 0.9 M/mL to 21 M/mL. In some embodiments, the density of hepatocytes is 1 M/mL to 20 M/mL. In some embodiments, the density of hepatocytes is 5 M/mL to 15 M/mL. In some embodiments, the density of hepatocytes is 10 M/mL.

In some embodiments, the density of hepatocytes is 1 M/mL to 12 M/mL (e.g., 2 M/mL to 11 M/mL, 3 M/mL to 10 M/mL, 4 M/mL to 9 M/mL, 5 M/mL to 8 M/mL, or 6 M/mL to 7 M/mL). For example, in some embodiments the density of hepatocytes is 2 M/mL to 11 M/mL. In some embodiments, the density of hepatocytes is 3 M/mL to 10 M/mL. In some embodiments, the density of hepatocytes is 4 M/mL to 9 M/mL. In some embodiments, the density of hepatocytes is 5 M/mL to 8 M/mL. In some embodiments, the density of hepatocytes is 6 M/mL to 7 M/mL.

In some embodiments, the density of hepatocytes is 3 M/mL to 12 M/mL (e.g., 4 M/mL to 11 M/mL, 5 M/mL to 10 M/mL, 6 M/mL to 9 M/mL, or 7 M/mL to 8 M/mL). For example, in some embodiments, the density of hepatocytes is 4 M/mL to 11 M/mL. In some embodiments, the density of hepatocytes is 5 M/mL to 10 M/mL. In some embodiments, the density of hepatocytes is 6 M/mL to 9 M/mL. In some embodiments, the density of hepatocytes is 7 M/mL to 8 M/mL.

In some embodiments, the density of hepatocytes is 0.1 M/mL. In some embodiments, the density of hepatocytes is 0.2 M/mL. In some embodiments, the density of hepatocytes is 0.3 M/mL. In some embodiments, the density of hepatocytes is 0.4 M/mL. In some embodiments, the density of hepatocytes is 0.5 M/mL. In some embodiments, the density of hepatocytes is 0.6 M/mL. In some embodiments, the density of hepatocytes is 0.7 M/mL. In some embodiments, the density of hepatocytes is 0.8 M/mL. In some embodiments, the density of hepatocytes is 0.9 M/mL. In some embodiments, the density of hepatocytes is 1 M/mL. In some embodiments, the density of hepatocytes is 2 M/mL. In some embodiments, the density of hepatocytes is 3 M/mL. In some embodiments, the density of hepatocytes is 4 M/mL. In some embodiments, the density of hepatocytes is 5 M/mL. In some embodiments, the density of hepatocytes is 6 M/mL. In some embodiments, the density of hepatocytes is 7 M/mL. In some embodiments, the density of hepatocytes is 8 M/mL. In some embodiments, the density of hepatocytes is 9 M/mL. In some embodiments, the density of hepatocytes is 10 M/mL. In some embodiments, the density of hepatocytes is 11 M/mL. In some embodiments, the density of hepatocytes is 12 M/mL. In some embodiments, the density of hepatocytes is 13 M/mL. In some embodiments, the density of hepatocytes is 14 M/mL. In some embodiments, the density of hepatocytes is 15 M/mL. In some embodiments, the density of hepatocytes is 16 M/mL. In some embodiments, the density of hepatocytes is 17 M/mL. In some embodiments, the density of hepatocytes is 18 M/mL. In some embodiments, the density of hepatocytes is 19 M/mL. In some embodiments, the density of hepatocytes is 20 M/mL. In some embodiments, the density of hepatocytes is 21 M/mL. In some embodiments, the density of hepatocytes is 22 M/mL. In some embodiments, the density of hepatocytes is 23 M/mL. In some embodiments, the density of hepatocytes is 24 M/mL. In some embodiments, the density of hepatocytes is 25 M/mL. In some embodiments, the density of hepatocytes is 26 M/mL. In some embodiments, the density of hepatocytes is 27 M/mL. In some embodiments, the density of hepatocytes is 28 M/mL. In some embodiments, the density of hepatocytes is 29 M/mL. In some embodiments, the density of hepatocytes is 30 M/mL. In some embodiments, the density of hepatocytes is 31 M/mL. In some embodiments, the density of hepatocytes is 32 M/mL. In some embodiments, the density of hepatocytes is 33 M/mL. In some embodiments, the density of hepatocytes is 34 M/mL. In some embodiments, the density of hepatocytes is 35 M/mL. In some embodiments, the density of hepatocytes is 36 M/mL. In some embodiments, the density of hepatocytes is 37 M/mL. In some embodiments, the density of hepatocytes is 38 M/mL. In some embodiments, the density of hepatocytes is 39 M/mL. In some embodiments, the density of hepatocytes is 40 M/mL. In some embodiments, the density of hepatocytes is 41 M/mL. In some embodiments, the density of hepatocytes is 42 M/mL. In some embodiments, the density of hepatocytes is 43 M/mL. In some embodiments, the density of hepatocytes is 44 M/mL. In some embodiments, the density of hepatocytes is 45 M/mL. In some embodiments, the density of hepatocytes is 46 M/mL. In some embodiments, the density of hepatocytes is 47 M/mL. In some embodiments, the density of hepatocytes is 48 M/mL. In some embodiments, the density of hepatocytes is 49 M/mL. In some embodiments, the density of hepatocytes is 50 M/mL. In some embodiments, the density of hepatocytes is 60 M/mL. In some embodiments, the density of hepatocytes is 70 M/mL. In some embodiments, the density of hepatocytes is 80 M/mL. In some embodiments, the density of hepatocytes is 90 M/mL. In some embodiments, the density of hepatocytes is 100 M/mL. In some embodiments, the density of hepatocytes is 150 M/mL.

In some embodiments, the hepatocytes have a quantifiable potency (e.g., an ability to decompose ammonia in a quantifiable range).

### I. Percentage of Hepatocytes Compared to Total Liver Mass

In some embodiments, the engineered tissue construct includes a population of hepatocytes in an amount that is equivalent to 0.5% to 30% (e.g., 0.5% to 30%, 0.6% to 30%, 0.7% to 30%, 0.8% to 30%, 0.9% to 30%, 1% to 30%, 2% to 30%, 3% to 30%, 4% to 30%, 5% to 30%, 10% to 30%, or 20% to 30%) of the total liver mass of the subject. For example, in some embodiments, the engineered tissue construct includes a population of hepatocytes in an amount that is equivalent to 0.6% to 30% of the total liver mass of the subject. In some embodiments, the engineered tissue construct includes a population of hepatocytes in an amount that is equivalent to 0.7% to 30% of the total liver mass of the subject. In some embodiments, the engineered tissue construct includes a population of hepatocytes in an amount that is equivalent to 0.8% to 30% of the total liver mass of the subject. In some embodiments, the engineered tissue construct includes a population of hepatocytes in an amount that is equivalent to 0.9% to 30% of the total liver mass of the subject. In some embodiments, the engineered tissue construct includes a population of hepatocytes in an amount that is equivalent to 1% to 30% of the total liver mass of the subject. In some embodiments, the engineered tissue construct includes a population of hepatocytes in an amount that is equivalent to 2% to 30% of the total liver mass of the subject. In some embodiments, the engineered tissue construct includes a population of hepatocytes in an amount that is equivalent to 3% to 30% of the total liver mass of the subject. In some embodiments, the engineered tissue construct includes a population of hepatocytes in an amount that is equivalent to 4% to 30% of the total liver mass of the subject. In some embodiments, the engineered tissue construct includes a population of hepatocytes in an amount that is equivalent to 5% to 30% of the total liver mass of the subject. In some embodiments, the engineered tissue construct includes a population of hepatocytes in an amount that is equivalent to 6% to 30% of the total liver mass of the subject. In some embodiments, the engineered tissue construct includes a population of hepatocytes in an amount that is equivalent to 7% to 30% of the total liver mass of the subject. In some embodiments, the engineered tissue construct includes a population of hepatocytes in an amount that is equivalent to 8% to 30% of the total liver mass of the subject. In some embodiments, the engineered tissue construct includes a population of hepatocytes in an amount that is equivalent to 9% to 30% of the total liver mass of the subject. In some embodiments, the engineered tissue construct includes a population of hepatocytes in an amount that is equivalent to 10% to 30% of the total liver mass of the subject. In some embodiments, the engineered tissue construct includes a population of hepatocytes in an amount that is equivalent to 11% to 30% of the total liver mass of the subject. In some embodiments, the engineered tissue construct includes a population of hepatocytes in an amount that is equivalent to 12% to 30% of the total liver mass of the subject. In some embodiments, the engineered tissue construct includes a population of hepatocytes in an amount that is equivalent to 13% to 30% of the total liver mass of the subject. In some embodiments, the engineered tissue construct includes a population of hepatocytes in an amount that is equivalent to 14% to 30% of the total liver mass of the subject. In some embodiments, the engineered tissue construct includes a population of hepatocytes in an amount that is equivalent to 15% to 30% of the total liver mass of the subject. In some embodiments, the engineered tissue construct includes a population of hepatocytes in an amount that is equivalent to 16% to 30% of the total liver mass of the subject. In some embodiments, the engineered tissue construct includes a population of hepatocytes in an amount that is equivalent to 17% to 30% of the total liver mass of the subject. In some embodiments, the engineered tissue construct includes a population of hepatocytes in an amount that is equivalent to 18% to 30% of the total liver mass of the subject. In some embodiments, the engineered tissue construct includes a population of hepatocytes in an amount that is equivalent to 19% to 30% of the total liver mass of the subject. In some embodiments, the engineered tissue construct includes a population of hepatocytes in an amount that is equivalent to 20% to 30% of the total liver mass of the subject. In some embodiments, the engineered tissue construct includes a population of hepatocytes in an amount that is equivalent to 21% to 30% of the total liver mass of the subject. In some embodiments, the engineered tissue construct includes a population of hepatocytes in an amount that is equivalent to 22% to 30% of the total liver mass of the subject. In some embodiments, the engineered tissue construct includes a population of hepatocytes in an amount that is equivalent to 23% to 30% of the total liver mass of the subject. In some embodiments, the engineered tissue construct includes a population of hepatocytes in an amount that is equivalent to 24% to 30% of the total liver mass of the subject. In some embodiments, the engineered tissue construct includes a population of hepatocytes in an amount that is equivalent to 25% to 30% of the total liver mass of the subject. In some embodiments, the engineered tissue construct includes a population of hepatocytes in an amount that is equivalent to 26% to 30% of the total liver mass of the subject. In some embodiments, the engineered tissue construct includes a population of hepatocytes in an amount that is equivalent to 27% to 30% of the total liver mass of the subject. In some embodiments, the engineered tissue construct includes a population of hepatocytes in an amount that is equivalent to 28% to 30% of the total liver mass of the subject. In some embodiments, the engineered tissue construct includes a population of hepatocytes in an amount that is equivalent to 29% to 30% of the total liver mass of the subject.

In some embodiments, the engineered tissue construct includes a population of hepatocytes in an amount that is equivalent to 0.5% of the total liver mass of the subject. For example, in some embodiments, the engineered tissue construct includes a population of hepatocytes in an amount that is equivalent to 0.6% of the total liver mass of the subject. In some embodiments, the engineered tissue construct includes a population of hepatocytes in an amount that is equivalent to 0.7% of the total liver mass of the subject. In some embodiments, the engineered tissue construct includes a population of hepatocytes in an amount that is equivalent to 0.8% of the total liver mass of the subject. In some embodiments, the engineered tissue construct includes a population of hepatocytes in an amount that is equivalent to 0.9% of the total liver mass of the subject. In some embodiments, the engineered tissue construct includes a population of hepatocytes in an amount that is equivalent to 1% of the total liver mass of the subject. In some embodiments, the engineered tissue construct includes a population of hepatocytes in an amount that is equivalent to 2% of the total liver mass of the subject. In some embodiments, the engineered tissue construct includes a population of hepatocytes in an amount that is equivalent to 3% of the total liver mass of the subject. In some embodiments, the engineered tissue construct includes a population of hepatocytes in an amount that is equivalent to 4% of the total liver mass of the subject. In some embodiments, the engineered tissue construct includes a population of hepatocytes in an amount that is equivalent to 5% of the total liver mass of the subject. In some embodiments, the engineered tissue construct includes a population of hepatocytes in an amount that is equivalent to 6% of the total liver mass of the subject. In some embodiments, the engineered tissue construct includes a population of hepatocytes in an amount that is equivalent to 7% of the total liver mass of the subject. In some embodiments, the engineered tissue construct includes a population of hepatocytes in an amount that is equivalent to 8% of the total liver mass of the subject. In some embodiments, the engineered tissue construct includes a population of hepatocytes in an amount that is equivalent to 9% of the total liver mass of the subject. In some embodiments, the engineered tissue construct includes a population of hepatocytes in an amount that is equivalent to 10% of the total liver mass of the subject. In some embodiments, the engineered tissue construct includes a population of hepatocytes in an amount that is equivalent to 11% of the total liver mass of the subject. In some embodiments, the engineered tissue construct includes a population of hepatocytes in an amount that is equivalent to 12% of the total liver mass of the subject. In some embodiments, the engineered tissue construct includes a population of hepatocytes in an amount that is equivalent to 13% of the total liver mass of the subject. In some embodiments, the engineered tissue construct includes a population of hepatocytes in an amount that is equivalent to 14% of the total liver mass of the subject. In some embodiments, the engineered tissue construct includes a population of hepatocytes in an amount that is equivalent to 15% of the total liver mass of the subject. In some embodiments, the engineered tissue construct includes a population of hepatocytes in an amount that is equivalent to 16% of the total liver mass of the subject. In some embodiments, the engineered tissue construct includes a population of hepatocytes in an amount that is equivalent to 17% of the total liver mass of the subject. In some embodiments, the engineered tissue construct includes a population of hepatocytes in an amount that is equivalent to 18% of the total liver mass of the subject. In some embodiments, the engineered tissue construct includes a population of hepatocytes in an amount that is equivalent to 19% of the total liver mass of the subject. In some embodiments, the engineered tissue construct includes a population of hepatocytes in an amount that is equivalent to 20% of the total liver mass of the subject. In some embodiments, the engineered tissue construct includes a population of hepatocytes in an amount that is equivalent to 21% of the total liver mass of the subject. In some embodiments, the engineered tissue construct includes a population of hepatocytes in an amount that is equivalent to 22% of the total liver mass of the subject. In some embodiments, the engineered tissue construct includes a population of hepatocytes in an amount that is equivalent to 23% of the total liver mass of the subject. In some embodiments, the engineered tissue construct includes a population of hepatocytes in an amount that is equivalent to 24% of the total liver mass of the subject. In some embodiments, the engineered tissue construct includes a population of hepatocytes in an amount that is equivalent to 25% of the total liver mass of the subject. In some embodiments, the engineered tissue construct includes a population of hepatocytes in an amount that is equivalent to 26% of the total liver mass of the subject. In some embodiments, the engineered tissue construct includes a population of hepatocytes in an amount that is equivalent to 27% of the total liver mass of the subject. In some embodiments, the engineered tissue construct includes a population of hepatocytes in an amount that is equivalent to 28% of the total liver mass of the subject. In some embodiments, the engineered tissue construct includes a population of hepatocytes in an amount that is equivalent to 29% of the total liver mass of the subject.

### II. Percentage of Hepatocytes Compared to Mass of Liver Reserve

In some embodiments, the engineered tissue construct includes a population of hepatocytes in an amount that is equivalent to 0.5% to 20% (e.g., 0.5% to 20%, 0.6% to 20%, 0.7% to 20%, 0.8% to 20%, 0.9% to 20%, 1% to 20%, 2% to 20%, 3% to 20%, 4% to 20%, 5% to 20%, or 10% to 20%) of the mass of the liver reserve of the subject. For example, in some embodiments, the engineered tissue construct includes a population of hepatocytes in an amount that is equivalent to 0.6% to 20% of the mass of the liver reserve of the subject. In some embodiments, the engineered tissue construct includes a population of hepatocytes in an amount that is equivalent to 0.7% to 20% of the mass of the liver reserve of the subject. In some embodiments, the engineered tissue construct includes a population of hepatocytes in an amount that is equivalent to 0.8% to 20% of the mass of the liver reserve of the subject. In some embodiments, the engineered tissue construct includes a population of hepatocytes in an amount that is equivalent to 0.9% to 20% of the mass of the liver reserve of the subject. In some embodiments, the engineered tissue construct includes a population of hepatocytes in an amount that is equivalent to 1% to 20% of the mass of the liver reserve of the subject. In some embodiments, the engineered tissue construct includes a population of hepatocytes in an amount that is equivalent to 2% to 20% of the mass of the liver reserve of the subject. In some embodiments, the engineered tissue construct includes a population of hepatocytes in an amount that is equivalent to 3% to 20% of the mass of the liver reserve of the subject. In some embodiments, the engineered tissue construct includes a population of hepatocytes in an amount that is equivalent to 4% to 20% of the mass of the liver reserve of the subject. In some embodiments, the engineered tissue construct includes a population of hepatocytes in an amount that is equivalent to 5% to 20% of the mass of the liver reserve of the subject. In some embodiments, the engineered tissue construct includes a population of hepatocytes in an amount that is equivalent to 6% to 20% of the mass of the liver reserve of the subject. In some embodiments, the engineered tissue construct includes a population of hepatocytes in an amount that is equivalent to 7% to 20% of the mass of the liver reserve of the subject. In some embodiments, the engineered tissue construct includes a population of hepatocytes in an amount that is equivalent to 8% to 20% of the mass of the liver reserve of the subject. In some embodiments, the engineered tissue construct includes a population of hepatocytes in an amount that is equivalent to 9% to 20% of the mass of the liver reserve of the subject. In some embodiments, the engineered tissue construct includes a population of hepatocytes in an amount that is equivalent to 10% to 20% of the mass of the liver reserve of the subject. In some embodiments, the engineered tissue construct includes a population of hepatocytes in an amount that is equivalent to 11% to 20% of the mass of the liver reserve of the subject. In some embodiments, the engineered tissue construct includes a population of hepatocytes in an amount that is equivalent to 12% to 20% of the mass of the liver reserve of the subject. In some embodiments, the engineered tissue construct includes a population of hepatocytes in an amount that is equivalent to 13% to 20% of the mass of the liver reserve of the subject. In some embodiments, the engineered tissue construct includes a population of hepatocytes in an amount that is equivalent to 14% to 20% of the mass of the liver reserve of the subject. In some embodiments, the engineered tissue construct includes a population of hepatocytes in an amount that is equivalent to 15% to 20% of the mass of the liver reserve of the subject. In some embodiments, the engineered tissue construct includes a population of hepatocytes in an amount that is equivalent to 16% to 20% of the mass of the liver reserve of the subject. In some embodiments, the engineered tissue construct includes a population of hepatocytes in an amount that is equivalent to 17% to 20% of the mass of the liver reserve of the subject. In some embodiments, the engineered tissue construct includes a population of hepatocytes in an amount that is equivalent to 18% to 20% of the mass of the liver reserve of the subject. In some embodiments, the engineered tissue construct includes a population of hepatocytes in an amount that is equivalent to 19% to 20% of the mass of the liver reserve of the subject.

In some embodiments, the engineered tissue construct includes a population of hepatocytes in an amount that is equivalent to 0.5% of the mass of the liver reserve of the subject. For example, in some embodiments, the engineered tissue construct includes a population of hepatocytes in an amount that is equivalent to 0.6% of the mass of the liver reserve of the subject. In some embodiments, the engineered tissue construct includes a population of hepatocytes in an amount that is equivalent to 0.7% of the mass of the liver reserve of the subject. In some embodiments, the engineered tissue construct includes a population of hepatocytes in an amount that is equivalent to 0.8% of the mass of the liver reserve of the subject. In some embodiments, the engineered tissue construct includes a population of hepatocytes in an amount that is equivalent to 0.9% of the mass of the liver reserve of the subject. In some embodiments, the engineered tissue construct includes a population of hepatocytes in an amount that is equivalent to 1% of the mass of the liver reserve of the subject. In some embodiments, the engineered tissue construct includes a population of hepatocytes in an amount that is equivalent to 2% of the mass of the liver reserve of the subject. In some embodiments, the engineered tissue construct includes a population of hepatocytes in an amount that is equivalent to 3% of the mass of the liver reserve of the subject. In some embodiments, the engineered tissue construct includes a population of hepatocytes in an amount that is equivalent to 4% of the mass of the liver reserve of the subject. In some embodiments, the engineered tissue construct includes a population of hepatocytes in an amount that is equivalent to 5% of the mass of the liver reserve of the subject. In some embodiments, the engineered tissue construct includes a population of hepatocytes in an amount that is equivalent to 6% of the mass of the liver reserve of the subject. In some embodiments, the engineered tissue construct includes a population of hepatocytes in an amount that is equivalent to 7% of the mass of the liver reserve of the subject. In some embodiments, the engineered tissue construct includes a population of hepatocytes in an amount that is equivalent to 8% of the mass of the liver reserve of the subject. In some embodiments, the engineered tissue construct includes a population of hepatocytes in an amount that is equivalent to 9% of the mass of the liver reserve of the subject. In some embodiments, the engineered tissue construct includes a population of hepatocytes in an amount that is equivalent to 10% of the mass of the liver reserve of the subject. In some embodiments, the engineered tissue construct includes a population of hepatocytes in an amount that is equivalent to 11% of the mass of the liver reserve of the subject. In some embodiments, the engineered tissue construct includes a population of hepatocytes in an amount that is equivalent to 12% of the mass of the liver reserve of the subject. In some embodiments, the engineered tissue construct includes a population of hepatocytes in an amount that is equivalent to 13% of the mass of the liver reserve of the subject. In some embodiments, the engineered tissue construct includes a population of hepatocytes in an amount that is equivalent to 14% of the mass of the liver reserve of the subject. In some embodiments, the engineered tissue construct includes a population of hepatocytes in an amount that is equivalent to 15% of the mass of the liver reserve of the subject. In some embodiments, the engineered tissue construct includes a population of hepatocytes in an amount that is equivalent to 16% of the mass of the liver reserve of the subject. In some embodiments, the engineered tissue construct includes a population of hepatocytes in an amount that is equivalent to 17% of the mass of the liver reserve of the subject. In some embodiments, the engineered tissue construct includes a population of hepatocytes in an amount that is equivalent to 18% of the mass of the liver reserve of the subject. In some embodiments, the engineered tissue construct includes a population of hepatocytes in an amount that is equivalent to 19% of the mass of the liver reserve of the subject. In some embodiments, the engineered tissue construct includes a population of hepatocytes in an amount that is equivalent to 20% of the mass of the liver reserve of the subject.

III. Number of hepatocytes in an engineered tissue construct

In some embodiments, the engineered tissue construct includes a population of hepatocytes in an amount of from about 3×10⁵ to about 1.8×10¹¹ (e.g., from about 4×10⁵ to about 1.8×10¹¹, from about 5× 10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes. For example, in some embodiments, the engineered tissue construct includes a population of hepatocytes in an amount of from about 3×10⁵ to about 1.8×10¹¹ hepatocytes. In some embodiments, the engineered tissue construct includes a population of hepatocytes in an amount of from about 4×10⁵ to about 1.8×10¹¹ hepatocytes. In some embodiments, the engineered tissue construct includes a population of hepatocytes in an amount of from about 5×10⁵ to about 1.8×10¹¹ hepatocytes. In some embodiments, the engineered tissue construct includes a population of hepatocytes in an amount of from about 6×10⁵ to about 1.8×10¹¹ hepatocytes. In some embodiments, the engineered tissue construct includes a population of hepatocytes in an amount of from about 7×10⁵ to about 1.8×10¹¹ hepatocytes. In some embodiments, the engineered tissue construct includes a population of hepatocytes in an amount of from about 8×10⁵ to about 1.8×10¹¹ hepatocytes. In some embodiments, the engineered tissue construct includes a population of hepatocytes in an amount of from about 9×10⁵ to about 1.8×10¹¹ hepatocytes. In some embodiments, the engineered tissue construct includes a population of hepatocytes in an amount of from about 1×10⁶ to about 1.8×10¹¹ hepatocytes. In some embodiments, the engineered tissue construct includes a population of hepatocytes in an amount of from about 2×10⁶ to about 1.8×10¹¹ hepatocytes. In some embodiments, the engineered tissue construct includes a population of hepatocytes in an amount of from about 3×10⁶ to about 1.8×10¹¹ hepatocytes. In some embodiments, the engineered tissue construct includes a population of hepatocytes in an amount of from about 4×10⁶ to about 1.8×10¹¹ hepatocytes. In some embodiments, the engineered tissue construct includes a population of hepatocytes in an amount of from about 5×10⁶ to about 1.8×10¹¹ hepatocytes. In some embodiments, the engineered tissue construct includes a population of hepatocytes in an amount of from about 6×10⁶ to about 1.8×10¹¹ hepatocytes. In some embodiments, the engineered tissue construct includes a population of hepatocytes in an amount of from about 7×10⁶ to about 1.8×10¹¹ hepatocytes. In some embodiments, the engineered tissue construct includes a population of hepatocytes in an amount of from about 8×10⁶ to about 1.8×10¹¹ hepatocytes. In some embodiments, the engineered tissue construct includes a population of hepatocytes in an amount of from about 9×10⁶ to about 1.8×10¹¹ hepatocytes. In some embodiments, the engineered tissue construct includes a population of hepatocytes in an amount of from about 1×10⁷ to about 1.8×10¹¹ hepatocytes. In some embodiments, the engineered tissue construct includes a population of hepatocytes in an amount of from about 2×10⁷ to about 1.8×10¹¹ hepatocytes. In some embodiments, the engineered tissue construct includes a population of hepatocytes in an amount of from about 3×10⁷ to about 1.8×10¹¹ hepatocytes. In some embodiments, the engineered tissue construct includes a population of hepatocytes in an amount of from about 4×10⁷ to about 1.8×10¹¹ hepatocytes. In some embodiments, the engineered tissue construct includes a population of hepatocytes in an amount of from about 5×10⁷ to about 1.8×10¹¹ hepatocytes. In some embodiments, the engineered tissue construct includes a population of hepatocytes in an amount of from about 6×10⁷ to about 1.8×10¹¹ hepatocytes. In some embodiments, the engineered tissue construct includes a population of hepatocytes in an amount of from about 7×10⁷ to about 1.8×10¹¹ hepatocytes. In some embodiments, the engineered tissue construct includes a population of hepatocytes in an amount of from about 8×10⁷ to about 1.8×10¹¹ hepatocytes. In some embodiments, the engineered tissue construct includes a population of hepatocytes in an amount of from about 9×10⁷ to about 1.8×10¹¹ hepatocytes. In some embodiments, the engineered tissue construct includes a population of hepatocytes in an amount of from about 1×10⁸ to about 1.8×10¹¹ hepatocytes. In some embodiments, the engineered tissue construct includes a population of hepatocytes in an amount of from about 2×10⁸ to about 1.8×10¹¹ hepatocytes. In some embodiments, the engineered tissue construct includes a population of hepatocytes in an amount of from about 3×10⁸ to about 1.8×10¹¹ hepatocytes. In some embodiments, the engineered tissue construct includes a population of hepatocytes in an amount of from about 4×10⁸ to about 1.8×10¹¹ hepatocytes. In some embodiments, the engineered tissue construct includes a population of hepatocytes in an amount of from about 5×10⁸ to about 1.8×10¹¹ hepatocytes. In some embodiments, the engineered tissue construct includes a population of hepatocytes in an amount of from about 6×10⁸ to about 1.8×10¹¹ hepatocytes. In some embodiments, the engineered tissue construct includes a population of hepatocytes in an amount of from about 7×10⁸ to about 1.8×10¹¹ hepatocytes. In some embodiments, the engineered tissue construct includes a population of hepatocytes in an amount of from about 8×10⁸ to about 1.8×10¹¹ hepatocytes. In some embodiments, the engineered tissue construct includes a population of hepatocytes in an amount of from about 9×10⁸ to about 1.8×10¹¹ hepatocytes. In some embodiments, the engineered tissue construct includes a population of hepatocytes in an amount of from about 1×10⁹ to about 1.8×10¹¹ hepatocytes. In some embodiments, the engineered tissue construct includes a population of hepatocytes in an amount of from about 2×10⁹ to about 1.8×10¹¹ hepatocytes. In some embodiments, the engineered tissue construct includes a population of hepatocytes in an amount of from about 3×10⁹ to about 1.8×10¹¹ hepatocytes. In some embodiments, the engineered tissue construct includes a population of hepatocytes in an amount of from about 4×10⁹ to about 1.8×10¹¹ hepatocytes. In some embodiments, the engineered tissue construct includes a population of hepatocytes in an amount of from about 5×10⁹ to about 1.8×10¹¹ hepatocytes. In some embodiments, the engineered tissue construct includes a population of hepatocytes in an amount of from about 6×10⁹ to about 1.8×10¹¹ hepatocytes. In some embodiments, the engineered tissue construct includes a population of hepatocytes in an amount of from about 7×10⁹ to about 1.8×10¹¹ hepatocytes. In some embodiments, the engineered tissue construct includes a population of hepatocytes in an amount of from about 8×10⁹ to about 1.8×10¹¹ hepatocytes. In some embodiments, the engineered tissue construct includes a population of hepatocytes in an amount of from about 9×10⁹ to about 1.8×10¹¹ hepatocytes. In some embodiments, the engineered tissue construct includes a population of hepatocytes in an amount of from about 1×10¹⁰ to about 1.8×10¹¹ hepatocytes. In some embodiments, the engineered tissue construct includes a population of hepatocytes in an amount of from about 2×10¹⁰ to about 1.8×10¹¹ hepatocytes. In some embodiments, the engineered tissue construct includes a population of hepatocytes in an amount of from about 3×10¹⁰ to about 1.8×10¹¹ hepatocytes. In some embodiments, the engineered tissue construct includes a population of hepatocytes in an amount of from about 4×10¹⁰ to about 1.8×10¹¹ hepatocytes. In some embodiments, the engineered tissue construct includes a population of hepatocytes in an amount of from about 5×10¹⁰ to about 1.8×10¹¹ hepatocytes. In some embodiments, the engineered tissue construct includes a population of hepatocytes in an amount of from about 6×10¹⁰ to about 1.8×10¹¹ hepatocytes. In some embodiments, the engineered tissue construct includes a population of hepatocytes in an amount of from about 7×10¹⁰ to about 1.8×10¹¹ hepatocytes. In some embodiments, the engineered tissue construct includes a population of hepatocytes in an amount of from about 8×10¹⁰ to about 1.8×10¹¹ hepatocytes. In some embodiments, the engineered tissue construct includes a population of hepatocytes in an amount of from about 9×10¹⁰ to about 1.8×10¹¹ hepatocytes. In some embodiments, the engineered tissue construct includes a population of hepatocytes in an amount of from about 1×10¹¹ to about 1.8×10¹¹ hepatocytes.

In some embodiments, the engineered tissue construct includes a population of hepatocytes in an amount of about 3×10⁵ hepatocytes. In some embodiments, the engineered tissue construct includes a population of hepatocytes in an amount of about 4×10⁵ hepatocytes. In some embodiments, the engineered tissue construct includes a population of hepatocytes in an amount of about 5×10⁵ hepatocytes. In some embodiments, the engineered tissue construct includes a population of hepatocytes in an amount of about 6×10⁵ hepatocytes. In some embodiments, the engineered tissue construct includes a population of hepatocytes in an amount of about 7×10⁵ hepatocytes. In some embodiments, the engineered tissue construct includes a population of hepatocytes in an amount of about 8×10⁵ hepatocytes. In some embodiments, the engineered tissue construct includes a population of hepatocytes in an amount of about 9×10⁵ hepatocytes. In some embodiments, the engineered tissue construct includes a population of hepatocytes in an amount of about 1×10⁶ hepatocytes. In some embodiments, the engineered tissue construct includes a population of hepatocytes in an amount of about 2×10⁶ hepatocytes. In some embodiments, the engineered tissue construct includes a population of hepatocytes in an amount of about 3×10⁶ hepatocytes. In some embodiments, the engineered tissue construct includes a population of hepatocytes in an amount of about 4×10⁶ hepatocytes. In some embodiments, the engineered tissue construct includes a population of hepatocytes in an amount of about 5×10⁶ hepatocytes. In some embodiments, the engineered tissue construct includes a population of hepatocytes in an amount of about 6×10⁶ hepatocytes. In some embodiments, the engineered tissue construct includes a population of hepatocytes in an amount of about 7×10⁶ hepatocytes. In some embodiments, the engineered tissue construct includes a population of hepatocytes in an amount of about 8×10⁶ hepatocytes. In some embodiments, the engineered tissue construct includes a population of hepatocytes in an amount of about 9×10⁶ hepatocytes. In some embodiments, the engineered tissue construct includes a population of hepatocytes in an amount of about 1×10⁷ hepatocytes. In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.8×10¹¹ hepatocytes.

Illa. Age-dependent number of hepatocytes in an engineered tissue construct Illai. Neonate (e.g., 0-30 days of age) In some embodiments, the engineered tissue construct is implanted in a neonate and includes a population of hepatocytes in an amount of from about 3×10⁵ to about 3×10¹⁰ (e.g., from about 4×10⁵ to about 3×10¹⁰, from about 5×10⁵ to about 3×10¹⁰, from about 6×10⁵ to about 3×10¹⁰, from about 7×10⁵ to about 3×10¹⁰, from about 8×10⁵ to about 3×10¹⁰, from about 9×10⁵ to about 3×10¹⁰, from about 1×10⁶ to about 3×10¹⁰, from about 2×10⁶ to about 3×10¹⁰, from about 3×10⁶ to about 3×10¹⁰, from about 4×10⁶ to about 3×10¹⁰, from about 5×10⁶ to about 3×10¹⁰, from about 6×10⁶ to about 3×10¹⁰, from about 7×10⁶ to about 3×10¹⁰, from about 8×10⁶ to about 3×10¹⁰, from about 9×10⁶ to about 3×10¹⁰, from about 1×10⁷ to about 3×10¹⁰, from about 2×10⁷ to about 3×10¹⁰, from about 3×10⁷ to about 3×10¹⁰, from about 4×10⁷ to about 3×10¹⁰, from about 5×10⁷ to about 3×10¹⁰, from about 6×10⁷ to about 3×10¹⁰, from about 7×10⁷ to about 3×10¹⁰, from about 8×10⁷ to about 3×10¹⁰, from about 9×10⁷ to about 3×10¹⁰, from about 1×10⁸ to about 3×10¹⁰, from about 2×10⁸ to about 3×10¹⁰, from about 3×10⁸ to about 3×10¹⁰, from about 4×10⁸ to about 3×10¹⁰, from about 5×10⁸ to about 3×10¹⁰, from about 6×10⁸ to about 3×10¹⁰, from about 7×10⁸ to about 3×10¹⁰, from about 8×10⁸ to about 3×10¹⁰, from about 9×10⁸ to about 3×10¹⁰, from about 1×10⁹ to about 3×10¹⁰, from about 2×10⁹ to about 3×10¹⁰, from about 3×10⁹ to about 3×10¹⁰, from about 4×10⁹ to about 3×10¹⁰, from about 5×10⁹ to about 3×10¹⁰, from about 6×10⁹ to about 3×10¹⁰, from about 7×10⁹ to about 3×10¹⁰, from about 8×10⁹ to about 3×10¹⁰, from about 9×10⁹ to about 3×10¹⁰, from about 1×10¹⁰ to about 3×10¹⁰, or from about 2×10¹⁰ to about 3×10¹⁰) hepatocytes. For example, in some embodiments, the engineered tissue construct includes a population of hepatocytes in an amount of from about 4×10⁵ to about 3×10¹⁰ hepatocytes. In some embodiments, the engineered tissue construct includes a population of hepatocytes in an amount of from about 5×10⁵ to about 3×10¹⁰ hepatocytes. In some embodiments, the engineered tissue construct includes a population of hepatocytes in an amount of from about 6×10⁵ to about 3×10¹⁰ hepatocytes. In some embodiments, the engineered tissue construct includes a population of hepatocytes in an amount of from about 7×10⁵ to about 3×10¹⁰ hepatocytes. In some embodiments, the engineered tissue construct includes a population of hepatocytes in an amount of from about 8×10⁵ to about 3×10¹⁰ hepatocytes. In some embodiments, the engineered tissue construct includes a population of hepatocytes in an amount of from about 9×10⁵ to about 3×10¹⁰ hepatocytes. In some embodiments, the engineered tissue construct includes a population of hepatocytes in an amount of from about 1×10⁶ to about 3×10¹⁰ hepatocytes. In some embodiments, the engineered tissue construct includes a population of hepatocytes in an amount of from about 2×10⁶ to about 3×10¹⁰ hepatocytes. In some embodiments, the engineered tissue construct includes a population of hepatocytes in an amount of from about 3×10⁶ to about 3×10¹⁰, from about 4×10⁶ to about 3×10¹⁰ hepatocytes. In some embodiments, the engineered tissue construct includes a population of hepatocytes in an amount of from about 5×10⁶ to about 3×10¹⁰ hepatocytes. In some embodiments, the engineered tissue construct includes a population of hepatocytes in an amount of from about 6×10⁶ to about 3×10¹⁰ hepatocytes. In some embodiments, the engineered tissue construct includes a population of hepatocytes in an amount of from about 7×10⁶ to about 3×10¹⁰ hepatocytes. In some embodiments, the engineered tissue construct includes a population of hepatocytes in an amount of from about 8×10⁶ to about 3×10¹⁰ hepatocytes. In some embodiments, the engineered tissue construct includes a population of hepatocytes in an amount of from about 9×10⁶ to about 3×10¹⁰ hepatocytes. In some embodiments, the engineered tissue construct includes a population of hepatocytes in an amount of 1×10⁷ to about 3×10¹⁰ hepatocytes. In some embodiments, the engineered tissue construct includes a population of hepatocytes in an amount of from about 2×10⁷ to about 3×10¹⁰ hepatocytes. In some embodiments, the engineered tissue construct includes a population of hepatocytes in an amount of from about 3×10⁷ to about 3×10¹⁰ hepatocytes. In some embodiments, the engineered tissue construct includes a population of hepatocytes in an amount of from about 4×10⁷ to about 3×10¹⁰ hepatocytes. In some embodiments, the engineered tissue construct includes a population of hepatocytes in an amount of from about 5×10⁷ to about 3×10¹⁰ hepatocytes. In some embodiments, the engineered tissue construct includes a population of hepatocytes in an amount of from about 6×10⁷ to about 3×10¹⁰ hepatocytes. In some embodiments, the engineered tissue construct includes a population of hepatocytes in an amount of from about 7×10⁷ to about 3×10¹⁰ hepatocytes. In some embodiments, the engineered tissue construct includes a population of hepatocytes in an amount of from about 8×10⁷ to about 3×10¹⁰ hepatocytes. In some embodiments, the engineered tissue construct includes a population of hepatocytes in an amount of from about 9×10⁷ to about 3×10¹⁰ hepatocytes. In some embodiments, the engineered tissue construct includes a population of hepatocytes in an amount of from about 1×10⁸ to about 3×10¹⁰ hepatocytes. In some embodiments, the engineered tissue construct includes a population of hepatocytes in an amount of from about 2×10⁸ to about 3×10¹⁰ hepatocytes. In some embodiments, the engineered tissue construct includes a population of hepatocytes in an amount of from about 3×10⁸ to about 3×10¹⁰ hepatocytes. In some embodiments, the engineered tissue construct includes a population of hepatocytes in an amount of from about 4×10⁸ to about 3×10¹⁰ hepatocytes. In some embodiments, the engineered tissue construct includes a population of hepatocytes in an amount of from about 5×10⁸ to about 3×10¹⁰ hepatocytes. In some embodiments, the engineered tissue construct includes a population of hepatocytes in an amount of from about 6×10⁸ to about 3×10¹⁰ hepatocytes. In some embodiments, the engineered tissue construct includes a population of hepatocytes in an amount of from about 7×10⁸ to about 3×10¹⁰ hepatocytes. In some embodiments, the engineered tissue construct includes a population of hepatocytes in an amount of from about 8×10⁸ to about 3×10¹⁰ hepatocytes. In some embodiments, the engineered tissue construct includes a population of hepatocytes in an amount of from about 9×10⁸ to about 3×10¹⁰ hepatocytes. In some embodiments, the engineered tissue construct includes a population of hepatocytes in an amount of from about 1×10⁹ to about 3×10¹⁰ hepatocytes. In some embodiments, the engineered tissue construct includes a population of hepatocytes in an amount of from about 2×10⁹ to about 3×10¹⁰ hepatocytes. In some embodiments, the engineered tissue construct includes a population of hepatocytes in an amount of from about 3×10⁹ to about 3×10¹⁰ hepatocytes. In some embodiments, the engineered tissue construct includes a population of hepatocytes in an amount of from about 4×10⁹ to about 3×10¹⁰ hepatocytes. In some embodiments, the engineered tissue construct includes a population of hepatocytes in an amount of from about 5×10⁹ to about 3×10¹⁰ hepatocytes. In some embodiments, the engineered tissue construct includes a population of hepatocytes in an amount of from about 6×10⁹ to about 3×10¹⁰ hepatocytes. In some embodiments, the engineered tissue construct includes a population of hepatocytes in an amount of from about 7×10⁹ to about 3×10¹⁰ hepatocytes. In some embodiments, the engineered tissue construct includes a population of hepatocytes in an amount of from about 8×10⁹ to about 3×10¹⁰ hepatocytes. In some embodiments, the engineered tissue construct includes a population of hepatocytes in an amount of from about 9×10⁹ to about 3×10¹⁰ hepatocytes. In some embodiments, the engineered tissue construct includes a population of hepatocytes in an amount of from about 1×10¹⁰ to about 3×10¹⁰ hepatocytes. In some embodiments, the engineered tissue construct includes a population of hepatocytes in an amount of from about 2×10¹⁰ to about 3×10¹⁰ hepatocytes. In some embodiments, the engineered tissue construct includes a population of hepatocytes in an amount of about 3×10⁵ hepatocytes. In some embodiments, the engineered tissue construct includes a population of hepatocytes in an amount of about 4×10⁵ hepatocytes. In some embodiments, the engineered tissue construct includes a population of hepatocytes in an amount of about 5×10⁵ hepatocytes. In some embodiments, the engineered tissue construct includes a population of hepatocytes in an amount of about 6×10⁵ hepatocytes. In some embodiments, the engineered tissue construct includes a population of hepatocytes in an amount of about 7×10⁵ hepatocytes. In some embodiments, the engineered tissue construct includes a population of hepatocytes in an amount of about 8×10⁵ hepatocytes. In some embodiments, the engineered tissue construct includes a population of hepatocytes in an amount of about 9×10⁵ hepatocytes. In some embodiments, the engineered tissue construct includes a population of hepatocytes in an amount of about 1×10⁶ hepatocytes. In some embodiments, the engineered tissue construct includes a population of hepatocytes in an amount of about 2×10⁶ hepatocytes. In some embodiments, the engineered tissue construct includes a population of hepatocytes in an amount of about 3×10⁶ hepatocytes. In some embodiments, the engineered tissue construct includes a population of hepatocytes in an amount of about 4×10⁶ hepatocytes. In some embodiments, the engineered tissue construct includes a population of hepatocytes in an amount of about 5×10⁶ hepatocytes. In some embodiments, the engineered tissue construct includes a population of hepatocytes in an amount of about 6×10⁶ hepatocytes. In some embodiments, the engineered tissue construct includes a population of hepatocytes in an amount of about 7×10⁶ hepatocytes. In some embodiments, the engineered tissue construct includes a population of hepatocytes in an amount of about 8×10⁶ hepatocytes. In some embodiments, the engineered tissue construct includes a population of hepatocytes in an amount of about 9×10⁶ hepatocytes. In some embodiments, the engineered tissue construct includes a population of hepatocytes in an amount of about 1×10⁷ hepatocytes. In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes.

Illaii. Infant (e.g., 1 year of age)

In some embodiments, the engineered tissue construct is implanted in an infant and includes a population of hepatocytes in an amount of from about 2×10⁷ to about 6×10¹⁰ (e.g., from about 3×10⁷ to about 6×10¹⁰, from about 4×10⁷ to about 6×10¹⁰, from about 5×10⁷ to about 6×10¹⁰, from about 6×10⁷ to about 6×10¹⁰, from about 7×10⁷ to about 6×10¹⁰, from about 8×10⁷ to about 6×10¹⁰, from about 9×10⁷ to about 6×10¹⁰, from about 1×10⁸ to about 6×10¹⁰, from about 2×10⁸ to about 6×10¹⁰, from about 3×10⁸ to about 6×10¹⁰, from about 4×10⁸ to about 6×10¹⁰, from about 5×10⁸ to about 6×10¹⁰, from about 6×10⁸ to about 6×10¹⁰, from about 7×10⁸ to about 6×10¹⁰, from about 8×10⁸ to about 6×10¹⁰, from about 9×10⁸ to about 6×10¹⁰, from about 1×10⁹ to about 6×10¹⁰, from about 2×10⁹ to about 6×10¹⁰, from about 3×10⁹ to about 6×10¹⁰, from about 4×10⁹ to about 6×10¹⁰, from about 5×10⁹ to about 6×10¹⁰, from about 6×10⁹ to about 6×10¹⁰, from about 7×10⁹ to about 6×10¹⁰, from about 8×10⁹ to about 6×10¹⁰, from about 9×10⁹ to about 6×10¹⁰, from about 1×10¹⁰ to about 6×10¹⁰, from about 2×10¹⁰ to about 6×10¹⁰. from about 3×10¹⁰ to about 6×10¹⁰, from about 4×10¹⁰ to about 6×10¹⁰, or from about 5×10¹⁰ to about 6×10¹⁰) hepatocytes. For example, in some embodiments, the engineered tissue construct includes a population of hepatocytes in an amount of from about 3×10⁷ to about 6×10¹⁰ hepatocytes. In some embodiments, the engineered tissue construct includes a population of hepatocytes in an amount of from about 4×10⁷ to about 6×10¹⁰ hepatocytes. In some embodiments, the engineered tissue construct includes a population of hepatocytes in an amount of from about 5×10⁷ to about 6×10¹⁰ hepatocytes. In some embodiments, the engineered tissue construct includes a population of hepatocytes in an amount of from about 6×10⁷ to about 6×10¹⁰ hepatocytes. In some embodiments, the engineered tissue construct includes a population of hepatocytes in an amount of from about 7×10⁷ to about 6×10¹⁰ hepatocytes. In some embodiments, the engineered tissue construct includes a population of hepatocytes in an amount of from about 8×10⁷ to about 6×10¹⁰ hepatocytes. In some embodiments, the engineered tissue construct includes a population of hepatocytes in an amount of from about 9×10⁷ to about 6×10¹⁰ hepatocytes. In some embodiments, the engineered tissue construct includes a population of hepatocytes in an amount of from about 1×10⁸ to about 6×10¹⁰ hepatocytes. In some embodiments, the engineered tissue construct includes a population of hepatocytes in an amount of from about 2×10⁸ to about 6×10¹⁰ hepatocytes. In some embodiments, the engineered tissue construct includes a population of hepatocytes in an amount of from about 3×10⁸ to about 6×10¹⁰ hepatocytes. In some embodiments, the engineered tissue construct includes a population of hepatocytes in an amount of from about 4×10⁸ to about 6×10¹⁰ hepatocytes. In some embodiments, the engineered tissue construct includes a population of hepatocytes in an amount of from about 5×10⁸ to about 6×10¹⁰ hepatocytes. In some embodiments, the engineered tissue construct includes a population of hepatocytes in an amount of from about 6×10⁸ to about 6×10¹⁰ hepatocytes. In some embodiments, the engineered tissue construct includes a population of hepatocytes in an amount of from about 7×10⁸ to about 6×10¹⁰ hepatocytes. In some embodiments, the engineered tissue construct includes a population of hepatocytes in an amount of from about 8×10⁸ to about 6×10¹⁰ hepatocytes. In some embodiments, the engineered tissue construct includes a population of hepatocytes in an amount of from about 9×10⁸ to about 6×10¹⁰ hepatocytes. In some embodiments, the engineered tissue construct includes a population of hepatocytes in an amount of from about 1×10⁹ to about 6×10¹⁰ hepatocytes. In some embodiments, the engineered tissue construct includes a population of hepatocytes in an amount of from about 2×10⁹ to about 6×10¹⁰ hepatocytes. In some embodiments, the engineered tissue construct includes a population of hepatocytes in an amount of from about 3×10⁹ to about 6×10¹⁰ hepatocytes. In some embodiments, the engineered tissue construct includes a population of hepatocytes in an amount of from about 4×10⁹ to about 6×10¹⁰ hepatocytes. In some embodiments, the engineered tissue construct includes a population of hepatocytes in an amount of from about 5×10⁹ to about 6×10¹⁰ hepatocytes. In some embodiments, the engineered tissue construct includes a population of hepatocytes in an amount of from about 6×10⁹ to about 6×10¹⁰ hepatocytes. In some embodiments, the engineered tissue construct includes a population of hepatocytes in an amount of from about 7×10⁹ to about 6×10¹⁰ hepatocytes. In some embodiments, the engineered tissue construct includes a population of hepatocytes in an amount of from about 8×10⁹ to about 6×10¹⁰ hepatocytes. In some embodiments, the engineered tissue construct includes a population of hepatocytes in an amount of from about 9×10⁹ to about 6×10¹⁰ hepatocytes. In some embodiments, the engineered tissue construct includes a population of hepatocytes in an amount of from about 1×10¹⁰ to about 6×10¹⁰ hepatocytes. In some embodiments, the engineered tissue construct includes a population of hepatocytes in an amount of from about 2×10¹⁰ to about 6×10¹⁰ hepatocytes. In some embodiments, the engineered tissue construct includes a population of hepatocytes in an amount of from about 3×10¹⁰ to about 6×10¹⁰ hepatocytes. In some embodiments, the engineered tissue construct includes a population of hepatocytes in an amount of from about 4×10¹⁰ to about 6×10¹⁰ hepatocytes. In some embodiments, the engineered tissue construct includes a population of hepatocytes in an amount of from about 5×10¹⁰ to about 6×10¹⁰ hepatocytes.

In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes.

Illaiii. Child (e.g., 5 years of age)

In some embodiments, the engineered tissue construct is implanted in a child and includes a population of hepatocytes in an amount of from about 3.5×10⁷ to about 1.05×10¹¹ (e.g., from about 4×10⁷ to about 1.05×10¹¹, from about 5×10⁷ to about 1.05×10¹¹, from about 6×10⁷ to about 1.05×10¹¹, from about 7×10⁷ to about 1.05×10¹¹, from about 8×10⁷ to about 1.05×10¹¹, from about 9×10⁷ to about 1.05×10¹¹, from about 1×10⁸ to about 1.05×10¹¹, from about 2×10⁸ to about 1.05×10¹¹, from about 3×10⁸ to about 1.05×10¹¹, from about 4×10⁸ to about 1.05×10¹¹, from about 5×10⁸ to about 1.05×10¹¹, from about 6×10⁸ to about 1.05×10¹¹, from about 7×10⁸ to about 1.05×10¹¹, from about 8×10⁸ to about 1.05×10¹¹, from about 9×10⁸ to about 1.05×10¹¹, from about 1×10⁹ to about 1.05×10¹¹, from about 2×10⁹ to about 1.05×10¹¹, from about 3×10⁹ to about 1.05×10¹¹, from about 4×10⁹ to about 1.05×10¹¹, from about 5×10⁹ to about 1.05×10¹¹, from about 6×10⁹ to about 1.05×10¹¹, from about 7×10⁹ to about 1.05×10¹¹, from about 8×10⁹ to about 1.05×10¹¹, from about 9×10⁹ to about 1.05×10¹¹, from about 1×10¹⁰ to about 1.05×10¹¹, from about 2×10¹⁰ to about 1.05×10¹¹, from about 3×10¹⁰ to about 1.05×10¹¹, from about 4×10¹⁰ to about 1.05×10¹¹, from about 5×10¹⁰ to about 1.05×10¹¹, from about 6×10¹⁰ to about 1.05×10¹¹, from about 7×10¹⁰ to about 1.05×10¹¹, from about 8×10¹⁰ to about 1.05×10¹¹, from about 9×10¹⁰ to about 1.05×10¹¹, from about 1×10¹¹ to about 1.05×10¹¹) hepatocytes. For example, in some embodiments, the engineered tissue construct includes a population of hepatocytes in an amount of from about 4×10⁷ to about 1.05×10¹¹ hepatocytes. In some embodiments, the engineered tissue construct includes a population of hepatocytes in an amount of from about 5×10⁷ to about 1.05×10¹¹ hepatocytes. In some embodiments, the engineered tissue construct includes a population of hepatocytes in an amount of from about 6×10⁷ to about 1.05×10¹¹ hepatocytes. In some embodiments, the engineered tissue construct includes a population of hepatocytes in an amount of from about 7×10⁷ to about 1.05×10¹¹. In some embodiments, the engineered tissue construct includes a population of hepatocytes in an amount of from about 8×10⁷ to about 1.05×10¹¹ hepatocytes. In some embodiments, the engineered tissue construct includes a population of hepatocytes in an amount of from about 9×10⁷ to about 1.05×10¹¹ hepatocytes. In some embodiments, the engineered tissue construct includes a population of hepatocytes in an amount of from about 1×10⁸ to about 1.05×10¹¹ hepatocytes. In some embodiments, the engineered tissue construct includes a population of hepatocytes in an amount of from about 2×10⁸ to about 1.05×10¹¹ hepatocytes. In some embodiments, the engineered tissue construct includes a population of hepatocytes in an amount of from about 3×10⁸ to about 1.05×10¹¹ hepatocytes. In some embodiments, the engineered tissue construct includes a population of hepatocytes in an amount of from about 4×10⁸ to about 1.05×10¹¹ hepatocytes. In some embodiments, the engineered tissue construct includes a population of hepatocytes in an amount of from about 5×10⁸ to about 1.05×10¹¹ hepatocytes. In some embodiments, the engineered tissue construct includes a population of hepatocytes in an amount of from about 6×10⁸ to about 1.05×10¹¹ hepatocytes. In some embodiments, the engineered tissue construct includes a population of hepatocytes in an amount of from about 7×10⁸ to about 1.05×10¹¹. In some embodiments, the engineered tissue construct includes a population of hepatocytes in an amount of from about 8×10⁸ to about 1.05×10¹¹ hepatocytes. In some embodiments, the engineered tissue construct includes a population of hepatocytes in an amount of from about 9×10⁸ to about 1.05×10¹¹ hepatocytes. In some embodiments, the engineered tissue construct includes a population of hepatocytes in an amount of from about 1×10⁹ to about 1.05×10¹¹ hepatocytes. In some embodiments, the engineered tissue construct includes a population of hepatocytes in an amount of from about 2×10⁹ to about 1.05×10¹¹ hepatocytes. In some embodiments, the engineered tissue construct includes a population of hepatocytes in an amount of from about 3×10⁹ to about 1.05×10¹¹ hepatocytes. In some embodiments, the engineered tissue construct includes a population of hepatocytes in an amount of from about 4×10⁹ to about 1.05×10¹¹ hepatocytes. In some embodiments, the engineered tissue construct includes a population of hepatocytes in an amount of from about 5×10⁹ to about 1.05×10¹¹ hepatocytes. In some embodiments, the engineered tissue construct includes a population of hepatocytes in an amount of from about 6×10⁹ to about 1.05×10¹¹ hepatocytes. In some embodiments, the engineered tissue construct includes a population of hepatocytes in an amount of from about 7×10⁹ to about 1.05×10¹¹. In some embodiments, the engineered tissue construct includes a population of hepatocytes in an amount of from about 8×10⁹ to about 1.05×10¹¹ hepatocytes. In some embodiments, the engineered tissue construct includes a population of hepatocytes in an amount of from about 9×10⁹ to about 1.05×10¹¹ hepatocytes. In some embodiments, the engineered tissue construct includes a population of hepatocytes in an amount of from about 1×10¹⁰ to about 1.05×10¹¹ hepatocytes. In some embodiments, the engineered tissue construct includes a population of hepatocytes in an amount of from about 2×10¹⁰ to about 1.05×10¹¹ hepatocytes. In some embodiments, the engineered tissue construct includes a population of hepatocytes in an amount of from about 3×10¹⁰ to about 1.05×10¹¹ hepatocytes. In some embodiments, the engineered tissue construct includes a population of hepatocytes in an amount of from about 4×10¹⁰ to about 1.05×10¹¹ hepatocytes. In some embodiments, the engineered tissue construct includes a population of hepatocytes in an amount of from about 5×10¹⁰ to about 1.05×10¹¹ hepatocytes. In some embodiments, the engineered tissue construct includes a population of hepatocytes in an amount of from about 6×10¹⁰ to about 1.05×10¹¹ hepatocytes. In some embodiments, the engineered tissue construct includes a population of hepatocytes in an amount of from about 7×10¹⁰ to about 1.05×10¹¹. In some embodiments, the engineered tissue construct includes a population of hepatocytes in an amount of from about 8×10¹⁰ to about 1.05×10¹¹ hepatocytes. In some embodiments, the engineered tissue construct includes a population of hepatocytes in an amount of from about 9×10¹⁰ to about 1.05×10¹¹ hepatocytes. In some embodiments, the engineered tissue construct includes a population of hepatocytes in an amount of from about 1×10¹¹ to about 1.05×10¹¹ hepatocytes.

In some embodiments, the engineered tissue construct includes a population of hepatocytes in an amount of about 3.5×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.05×10¹¹ hepatocytes.

Illaiv. Child (e.g., 9 Years of Age)

In some embodiments, the engineered tissue construct is implanted in a child and includes a population of hepatocytes in an amount of from about 4.5×10⁷ to about 1.35×10¹¹ (e.g., from about 5×10⁷ to about 1.35×10¹¹, from about 6×10⁷ to about 1.35×10¹¹, from about 7×10⁷ to about 1.35×10¹¹, from about 8×10⁷ to about 1.35×10¹¹, from about 9×10⁷ to about 1.35×10¹¹, from about 1×10⁸ to about 1.35×10¹¹, from about 2×10⁸ to about 1.35×10¹¹, from about 3×10⁸ to about 1.35×10¹¹, from about 4×10⁸ to about 1.35×10¹¹, from about 5×10⁸ to about 1.35×10¹¹, from about 6×10⁸ to about 1.35×10¹¹, from about 7×10⁸ to about 1.35×10¹¹, from about 8×10⁸ to about 1.35×10¹¹, from about 9×10⁸ to about 1.35×10¹¹, from about 1×10⁹ to about 1.35×10¹¹, from about 2×10⁹ to about 1.35×10¹¹, from about 3×10⁹ to about 1.35×10¹¹, from about 4×10⁹ to about 1.35×10¹¹, from about 5×10⁹ to about 1.35×10¹¹, from about 6×10⁹ to about 1.35×10¹¹, from about 7×10⁹ to about 1.35×10¹¹, from about 8×10⁹ to about 1.35×10¹¹, from about 9×10⁹ to about 1.35×10¹¹, from about 1×10¹⁰ to about 1.35×10¹¹, from about 2×10¹⁰ to about 1.35×10¹¹, from about 3×10¹⁰ to about 1.35×10¹¹, from about 4×10¹⁰ to about 1.35×10¹¹, from about 5×10¹⁰ to about 1.35×10¹¹, from about 6×10¹⁰ to about 1.35×10¹¹, from about 7×10¹⁰ to about 1.35×10¹¹, from about 8×10¹⁰ to about 1.35×10¹¹, from about 9×10¹⁰ to about 1.35×10¹¹, from about 1×10¹¹ to about 1.35×10¹¹) hepatocytes. For example, in some embodiments, the engineered tissue construct includes a population of hepatocytes in an amount of from about 4×10⁷ to about 1.35×10¹¹ hepatocytes. In some embodiments, the engineered tissue construct includes a population of hepatocytes in an amount of from about 5×10⁷ to about 1.35×10¹¹ hepatocytes. In some embodiments, the engineered tissue construct includes a population of hepatocytes in an amount of from about 6×10⁷ to about 1.35×10¹¹ hepatocytes. In some embodiments, the engineered tissue construct includes a population of hepatocytes in an amount of from about 7×10⁷ to about 1.35×10¹¹. In some embodiments, the engineered tissue construct includes a population of hepatocytes in an amount of from about 8×10⁷ to about 1.35×10¹¹ hepatocytes. In some embodiments, the engineered tissue construct includes a population of hepatocytes in an amount of from about 9×10⁷ to about 1.35×10¹¹ hepatocytes. In some embodiments, the engineered tissue construct includes a population of hepatocytes in an amount of from about 1×10⁸ to about 1.35×10¹¹ hepatocytes. In some embodiments, the engineered tissue construct includes a population of hepatocytes in an amount of from about 2×10⁸ to about 1.35×10¹¹ hepatocytes. In some embodiments, the engineered tissue construct includes a population of hepatocytes in an amount of from about 3×10⁸ to about 1.35×10¹¹ hepatocytes. In some embodiments, the engineered tissue construct includes a population of hepatocytes in an amount of from about 4×10⁸ to about 1.35×10¹¹ hepatocytes. In some embodiments, the engineered tissue construct includes a population of hepatocytes in an amount of from about 5×10⁸ to about 1.35×10¹¹ hepatocytes. In some embodiments, the engineered tissue construct includes a population of hepatocytes in an amount of from about 6×10⁸ to about 1.35×10¹¹ hepatocytes. In some embodiments, the engineered tissue construct includes a population of hepatocytes in an amount of from about 7×10⁸ to about 1.35×10¹¹. In some embodiments, the engineered tissue construct includes a population of hepatocytes in an amount of from about 8×10⁸ to about 1.35×10¹¹ hepatocytes. In some embodiments, the engineered tissue construct includes a population of hepatocytes in an amount of from about 9×10⁸ to about 1.35×10¹¹ hepatocytes. In some embodiments, the engineered tissue construct includes a population of hepatocytes in an amount of from about 1×10⁹ to about 1.35×10¹¹ hepatocytes. In some embodiments, the engineered tissue construct includes a population of hepatocytes in an amount of from about 2×10⁹ to about 1.35×10¹¹ hepatocytes. In some embodiments, the engineered tissue construct includes a population of hepatocytes in an amount of from about 3×10⁹ to about 1.35×10¹¹ hepatocytes. In some embodiments, the engineered tissue construct includes a population of hepatocytes in an amount of from about 4×10⁹ to about 1.35×10¹¹ hepatocytes. In some embodiments, the engineered tissue construct includes a population of hepatocytes in an amount of from about 5×10⁹ to about 1.35×10¹¹ hepatocytes. In some embodiments, the engineered tissue construct includes a population of hepatocytes in an amount of from about 6×10⁹ to about 1.35×10¹¹ hepatocytes. In some embodiments, the engineered tissue construct includes a population of hepatocytes in an amount of from about 7×10⁹ to about 1.35×10¹¹. In some embodiments, the engineered tissue construct includes a population of hepatocytes in an amount of from about 8×10⁹ to about 1.35×10¹¹ hepatocytes. In some embodiments, the engineered tissue construct includes a population of hepatocytes in an amount of from about 9×10⁹ to about 1.35×10¹¹ hepatocytes. In some embodiments, the engineered tissue construct includes a population of hepatocytes in an amount of from about 1×10¹⁰ to about 1.35×10¹¹ hepatocytes. In some embodiments, the engineered tissue construct includes a population of hepatocytes in an amount of from about 2×10¹⁰ to about 1.35×10¹¹ hepatocytes. In some embodiments, the engineered tissue construct includes a population of hepatocytes in an amount of from about 3×10¹⁰ to about 1.35×10¹¹ hepatocytes. In some embodiments, the engineered tissue construct includes a population of hepatocytes in an amount of from about 4×10¹⁰ to about 1.35×10¹¹ hepatocytes. In some embodiments, the engineered tissue construct includes a population of hepatocytes in an amount of from about 5×10¹⁰ to about 1.35×10¹¹ hepatocytes. In some embodiments, the engineered tissue construct includes a population of hepatocytes in an amount of from about 6×10¹⁰ to about 1.35×10¹¹ hepatocytes. In some embodiments, the engineered tissue construct includes a population of hepatocytes in an amount of from about 7×10¹⁰ to about 1.35×10¹¹. In some embodiments, the engineered tissue construct includes a population of hepatocytes in an amount of from about 8×10¹⁰ to about 1.35×10¹¹ hepatocytes. In some embodiments, the engineered tissue construct includes a population of hepatocytes in an amount of from about 9×10¹⁰ to about 1.35×10¹¹ hepatocytes. In some embodiments, the engineered tissue construct includes a population of hepatocytes in an amount of from about 1×10¹¹ to about 1.35×10¹¹ hepatocytes.

In some embodiments, the engineered tissue construct includes a population of hepatocytes in an amount of about 3.5×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.35×10¹¹ hepatocytes.

Illav. Adult (e.g., 18 Years of Age and Older)

In some embodiments, the engineered tissue construct is implanted in an adult and includes a population of hepatocytes in an amount of from about 9×10⁷ to about 1.8×10¹¹ (e.g., from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes. For example, in some embodiments, the engineered tissue construct includes a population of hepatocytes in an amount of from about 4×10⁷ to about 1.8×10¹¹ hepatocytes. In some embodiments, the engineered tissue construct includes a population of hepatocytes in an amount of from about 5×10⁷ to about 1.8×10¹¹ hepatocytes. In some embodiments, the engineered tissue construct includes a population of hepatocytes in an amount of from about 6×10⁷ to about 1.8×10¹¹ hepatocytes. In some embodiments, the engineered tissue construct includes a population of hepatocytes in an amount of from about 7×10⁷ to about 1.8×10¹¹. In some embodiments, the engineered tissue construct includes a population of hepatocytes in an amount of from about 8×10⁷ to about 1.8×10¹¹ hepatocytes. In some embodiments, the engineered tissue construct includes a population of hepatocytes in an amount of from about 9×10⁷ to about 1.8×10¹¹ hepatocytes. In some embodiments, the engineered tissue construct includes a population of hepatocytes in an amount of from about 1×10⁸ to about 1.8×10¹¹ hepatocytes. In some embodiments, the engineered tissue construct includes a population of hepatocytes in an amount of from about 2×10⁸ to about 1.8×10¹¹ hepatocytes. In some embodiments, the engineered tissue construct includes a population of hepatocytes in an amount of from about 3×10⁸ to about 1.8×10¹¹ hepatocytes. In some embodiments, the engineered tissue construct includes a population of hepatocytes in an amount of from about 4×10⁸ to about 1.8×10¹¹ hepatocytes. In some embodiments, the engineered tissue construct includes a population of hepatocytes in an amount of from about 5×10⁸ to about 1.8×10¹¹ hepatocytes. In some embodiments, the engineered tissue construct includes a population of hepatocytes in an amount of from about 6×10⁸ to about 1.8×10¹¹ hepatocytes. In some embodiments, the engineered tissue construct includes a population of hepatocytes in an amount of from about 7×10⁸ to about 1.8×10¹¹. In some embodiments, the engineered tissue construct includes a population of hepatocytes in an amount of from about 8×10⁸ to about 1.8×10¹¹ hepatocytes. In some embodiments, the engineered tissue construct includes a population of hepatocytes in an amount of from about 9×10⁸ to about 1.8×10¹¹ hepatocytes. In some embodiments, the engineered tissue construct includes a population of hepatocytes in an amount of from about 1×10⁹ to about 1.8×10¹¹ hepatocytes. In some embodiments, the engineered tissue construct includes a population of hepatocytes in an amount of from about 2×10⁹ to about 1.8×10¹¹ hepatocytes. In some embodiments, the engineered tissue construct includes a population of hepatocytes in an amount of from about 3×10⁹ to about 1.8×10¹¹ hepatocytes. In some embodiments, the engineered tissue construct includes a population of hepatocytes in an amount of from about 4×10⁹ to about 1.8×10¹¹ hepatocytes. In some embodiments, the engineered tissue construct includes a population of hepatocytes in an amount of from about 5×10⁹ to about 1.8×10¹¹ hepatocytes. In some embodiments, the engineered tissue construct includes a population of hepatocytes in an amount of from about 6×10⁹ to about 1.8×10¹¹ hepatocytes. In some embodiments, the engineered tissue construct includes a population of hepatocytes in an amount of from about 7×10⁹ to about 1.8×10¹¹. In some embodiments, the engineered tissue construct includes a population of hepatocytes in an amount of from about 8×10⁹ to about 1.8×10¹¹ hepatocytes. In some embodiments, the engineered tissue construct includes a population of hepatocytes in an amount of from about 9×10⁹ to about 1.8×10¹¹ hepatocytes. In some embodiments, the engineered tissue construct includes a population of hepatocytes in an amount of from about 1×10¹⁰ to about 1.8×10¹¹ hepatocytes. In some embodiments, the engineered tissue construct includes a population of hepatocytes in an amount of from about 2×10¹⁰ to about 1.8×10¹¹ hepatocytes. In some embodiments, the engineered tissue construct includes a population of hepatocytes in an amount of from about 3×10¹⁰ to about 1.8×10¹¹ hepatocytes. In some embodiments, the engineered tissue construct includes a population of hepatocytes in an amount of from about 4×10¹⁰ to about 1.8×10¹¹ hepatocytes. In some embodiments, the engineered tissue construct includes a population of hepatocytes in an amount of from about 5×10¹⁰ to about 1.8×10¹¹ hepatocytes. In some embodiments, the engineered tissue construct includes a population of hepatocytes in an amount of from about 6×10¹⁰ to about 1.8×10¹¹ hepatocytes. In some embodiments, the engineered tissue construct includes a population of hepatocytes in an amount of from about 7×10¹⁰ to about 1.8×10¹¹. In some embodiments, the engineered tissue construct includes a population of hepatocytes in an amount of from about 8×10¹⁰ to about 1.8×10¹¹ hepatocytes. In some embodiments, the engineered tissue construct includes a population of hepatocytes in an amount of from about 9×10¹⁰ to about 1.8×10¹¹ hepatocytes. In some embodiments, the engineered tissue construct includes a population of hepatocytes in an amount of from about 1×10¹¹ to about 1.8×10¹¹ hepatocytes.

In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.8×10¹¹ hepatocytes.

### Stromal Cells

The engineered tissue constructs described herein optionally include stromal cells. In some embodiments, the stromal cells are fibroblasts. In some embodiments, the fibroblasts are human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). In some embodiments, the fibroblasts are human dermal fibroblasts. In some embodiments, the fibroblasts are normal human dermal fibroblasts. In some embodiments, the fibroblasts are neonatal foreskin fibroblasts. In some embodiments, the fibroblasts human lung fibroblasts. In some embodiments, the fibroblasts are human ventricular cardiac fibroblasts. In some embodiments, the fibroblasts are human atrial cardiac fibroblasts. In some embodiments, the fibroblasts are human uterine fibroblasts. In some embodiments, the fibroblasts are human bladder fibroblasts. In some embodiments, the fibroblasts are human gingival fibroblasts. In some embodiments, the fibroblasts are human pericardial fibroblasts. In some embodiments, the fibroblasts are human gall bladder fibroblasts. In some embodiments, the fibroblasts are human portal vein fibroblasts. In some embodiments, the fibroblasts are vas deferens fibroblasts. In some embodiments the engineered tissue construct includes an optional population of stromal cells (e.g., fibroblasts) in an amount that is effective to treat ALF in a subject.

In some embodiments the engineered tissue construct optionally includes a population of stromal cells (e.g., fibroblasts) in an amount that is effective to treat hyperbilirubinemia in a subject.

In some embodiments the engineered tissue construct includes an optional population of stromal cells (e.g., fibroblasts) in an amount that is effective to reduce bilirubin levels in the subject.

In some embodiments, the population of optional stromal cells (e.g., fibroblasts) is up to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², 3×10⁶ to about 1.8×10¹², 4×10⁶ to about 1.8×10¹², 5×10⁶ to about 1.8×10¹², 6×10⁶ to about 1.8×10¹², 7×10⁶ to about 1.8×10¹², 8×10⁶ to about 1.8×10¹², 9×10⁶ to about 1.8×10¹², from about 1×10⁷ to about 1.8×10¹², from about 2×10⁷ to about 1.8×10¹², from about 18×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7× 10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts). For example, in some embodiments, the engineered tissue construct includes a population of stromal cells (e.g., fibroblasts) in an amount of from about 1 to about 1.8×10¹² stromal cells (e.g., fibroblasts). In some embodiments, the engineered tissue construct includes a population of stromal cells (e.g., fibroblasts) in an amount of from about 10 to about 1.8×10¹² stromal cells (e.g., fibroblasts). In some embodiments, the engineered tissue construct includes a population of stromal cells (e.g., fibroblasts) in an amount of from about 100 to about 1.8×10¹² stromal cells (e.g., fibroblasts). In some embodiments, the engineered tissue construct includes a population of stromal cells (e.g., fibroblasts) in an amount of from about 1×10³ to about 1.8×10¹² stromal cells (e.g., fibroblasts). In some embodiments, the engineered tissue construct includes a population of stromal cells (e.g., fibroblasts) in an amount from about 2×10³ to about 1.8×10¹² stromal cells (e.g., fibroblasts). In some embodiments, the engineered tissue construct includes a population of stromal cells (e.g., fibroblasts) in an amount of from about 3×10³ to about 1.8×10¹² stromal cells (e.g., fibroblasts). In some embodiments, the engineered tissue construct includes a population of stromal cells (e.g., fibroblasts) in an amount of from about 4×10³ to about 1.8×10¹² stromal cells (e.g., fibroblasts). In some embodiments, the engineered tissue construct includes a population of stromal cells (e.g., fibroblasts) in an amount of from about 5×10³ to about 1.8×10¹² stromal cells (e.g., fibroblasts). In some embodiments, the engineered tissue construct includes a population of stromal cells (e.g., fibroblasts) in an amount of from about 6×10³ to about 1.8×10¹² stromal cells (e.g., fibroblasts). In some embodiments, the engineered tissue construct includes a population of stromal cells (e.g., fibroblasts) in an amount of from about 7×10³ to about 1.8×10¹² stromal cells (e.g., fibroblasts). In some embodiments, the engineered tissue construct includes a population of stromal cells (e.g., fibroblasts) in an amount of from about 8×10³ to about 1.8×10¹² stromal cells (e.g., fibroblasts). In some embodiments, the engineered tissue construct includes a population of stromal cells (e.g., fibroblasts) in an amount of from about 9×10³ to about 1.8×10¹² stromal cells (e.g., fibroblasts). In some embodiments, the engineered tissue construct includes a population of stromal cells (e.g., fibroblasts) in an amount of from about 1×10⁴ to about 1.8×10¹² stromal cells (e.g., fibroblasts). In some embodiments, the engineered tissue construct includes a population of stromal cells (e.g., fibroblasts) in an amount of from about 2×10⁴ to about 1.8×10¹² stromal cells (e.g., fibroblasts). In some embodiments, the engineered tissue construct includes a population of stromal cells (e.g., fibroblasts) in an amount of from about 3×10⁴ to about 1.8×10¹² stromal cells (e.g., fibroblasts). In some embodiments, the engineered tissue construct includes a population of stromal cells (e.g., fibroblasts) in an amount of from about 4×10⁴ to about 1.8×10¹² stromal cells (e.g., fibroblasts). In some embodiments, the engineered tissue construct includes a population of stromal cells (e.g., fibroblasts) in an amount of from about 5×10⁴ to about 1.8×10¹² stromal cells (e.g., fibroblasts). In some embodiments, the engineered tissue construct includes a population of stromal cells (e.g., fibroblasts) in an amount of from about 6×10⁴ to about 1.8×10¹² stromal cells (e.g., fibroblasts). In some embodiments, the engineered tissue construct includes a population of stromal cells (e.g., fibroblasts) in an amount of from about 7×10⁴ to about 1.8×10¹² stromal cells (e.g., fibroblasts). In some embodiments, the engineered tissue construct includes a population of stromal cells (e.g., fibroblasts) in an amount of from about 8×10⁴ to about 1.8×10¹² stromal cells (e.g., fibroblasts). In some embodiments, the engineered tissue construct includes a population of stromal cells (e.g., fibroblasts) in an amount of from about 9×10⁴ to about 1.8×10¹² stromal cells (e.g., fibroblasts). In some embodiments, the engineered tissue construct includes a population of stromal cells (e.g., fibroblasts) in an amount of from about 1×10⁵ to about 1.8×10¹² stromal cells (e.g., fibroblasts). In some embodiments, the engineered tissue construct includes a population of stromal cells (e.g., fibroblasts) in an amount of from about 2×10⁵ to about 1.8×10¹² stromal cells (e.g., fibroblasts). In some embodiments, the engineered tissue construct includes a population of stromal cells (e.g., fibroblasts) in an amount of from about 3×10⁵ to about 1.8×10¹² stromal cells (e.g., fibroblasts). In some embodiments, the engineered tissue construct includes a population of stromal cells (e.g., fibroblasts) in an amount of from about 4×10⁵ to about 1.8×10¹² stromal cells (e.g., fibroblasts). In some embodiments, the engineered tissue construct includes a population of stromal cells (e.g., fibroblasts) in an amount of from about 5×10⁵ to about 1.8×10¹² stromal cells (e.g., fibroblasts). In some embodiments, the engineered tissue construct includes a population of stromal cells (e.g., fibroblasts) in an amount of from about 6×10⁵ to about 1.8×10¹² stromal cells (e.g., fibroblasts). In some embodiments, the engineered tissue construct includes a population of stromal cells (e.g., fibroblasts) in an amount of from about 7×10⁵ to about 1.8×10¹² stromal cells (e.g., fibroblasts). In some embodiments, the engineered tissue construct includes a population of stromal cells (e.g., fibroblasts) in an amount of from about 8×10⁵ to about 1.8×10¹² stromal cells (e.g., fibroblasts). In some embodiments, the engineered tissue construct includes a population of stromal cells (e.g., fibroblasts) in an amount of from about 9×10⁵ to about 1.8×10¹² stromal cells (e.g., fibroblasts). In some embodiments, the engineered tissue construct includes a population of stromal cells (e.g., fibroblasts) in an amount of from about 1×10⁶ to about 1.8×10¹² stromal cells (e.g., fibroblasts). In some embodiments, the engineered tissue construct includes a population of stromal cells (e.g., fibroblasts) in an amount of from about 2×10⁶ to about 1.8×10¹² stromal cells (e.g., fibroblasts). In some embodiments, the engineered tissue construct includes a population of stromal cells (e.g., fibroblasts) in an amount of from about 3×10⁶ to about 1.8×10¹² stromal cells (e.g., fibroblasts). In some embodiments, the engineered tissue construct includes a population of stromal cells (e.g., fibroblasts) in an amount of from about 4×10⁶ to about 1.8×10¹² stromal cells (e.g., fibroblasts). In some embodiments, the engineered tissue construct includes a population of stromal cells (e.g., fibroblasts) in an amount of from about 5×10⁶ to about 1.8×10¹² stromal cells (e.g., fibroblasts). In some embodiments, the engineered tissue construct includes a population of stromal cells (e.g., fibroblasts) in an amount of from about 6×10⁶ to about 1.8×10¹² stromal cells (e.g., fibroblasts). In some embodiments, the engineered tissue construct includes a population of stromal cells (e.g., fibroblasts) in an amount of from about 7×10⁶ to about 1.8×10¹² stromal cells (e.g., fibroblasts). In some embodiments, the engineered tissue construct includes a population of stromal cells (e.g., fibroblasts) in an amount of from about 8×10⁶ to about 1.8×10¹² stromal cells (e.g., fibroblasts). In some embodiments, the engineered tissue construct includes a population of stromal cells (e.g., fibroblasts) in an amount of from about 9×10⁶ to about 1.8×10¹² stromal cells (e.g., fibroblasts). In some embodiments, the engineered tissue construct includes a population of stromal cells (e.g., fibroblasts) in an amount of from about 1×10⁷ to about 1.8×10¹² stromal cells (e.g., fibroblasts). In some embodiments, the engineered tissue construct includes a population of stromal cells (e.g., fibroblasts) in an amount of from about 2×10⁷ to about 1.8×10¹² stromal cells (e.g., fibroblasts). In some embodiments, the engineered tissue construct includes a population of stromal cells (e.g., fibroblasts) in an amount of from about 3×10⁷ to about 1.8×10¹² stromal cells (e.g., fibroblasts). In some embodiments, the engineered tissue construct includes a population of stromal cells (e.g., fibroblasts) in an amount of from about 4×10⁷ to about 1.8×10¹² stromal cells (e.g., fibroblasts). In some embodiments, the engineered tissue construct includes a population of stromal cells (e.g., fibroblasts) in an amount of from about 5×10⁷ to about 1.8×10¹² stromal cells (e.g., fibroblasts). In some embodiments, the engineered tissue construct includes a population of stromal cells (e.g., fibroblasts) in an amount of from about 6×10⁷ to about 1.8×10¹² stromal cells (e.g., fibroblasts). In some embodiments, the engineered tissue construct includes a population of stromal cells (e.g., fibroblasts) in an amount of from about 7×10⁷ to about 1.8×10¹² stromal cells (e.g., fibroblasts). In some embodiments, the engineered tissue construct includes a population of stromal cells (e.g., fibroblasts) in an amount of from about 8×10⁷ to about 1.8×10¹² stromal cells (e.g., fibroblasts). In some embodiments, the engineered tissue construct includes a population of stromal cells (e.g., fibroblasts) in an amount of from about 9×10⁷ to about 1.8×10¹² stromal cells (e.g., fibroblasts). In some embodiments, the engineered tissue construct includes a population of stromal cells (e.g., fibroblasts) in an amount of from about 1×10⁸ to about 1.8×10¹² stromal cells (e.g., fibroblasts). In some embodiments, the engineered tissue construct includes a population of stromal cells (e.g., fibroblasts) in an amount of from about 2×10⁸ to about 1.8×10¹² stromal cells (e.g., fibroblasts). In some embodiments, the engineered tissue construct includes a population of stromal cells (e.g., fibroblasts) in an amount of from about 3×10⁸ to about 1.8×10¹² stromal cells (e.g., fibroblasts). In some embodiments, the engineered tissue construct includes a population of stromal cells (e.g., fibroblasts) in an amount of from about 4×10⁸ to about 1.8×10¹² stromal cells (e.g., fibroblasts). In some embodiments, the engineered tissue construct includes a population of stromal cells (e.g., fibroblasts) in an amount of from about 5×10⁸ to about 1.8×10¹² stromal cells (e.g., fibroblasts). In some embodiments, the engineered tissue construct includes a population of stromal cells (e.g., fibroblasts) in an amount of from about 6×10⁸ to about 1.8×10¹² stromal cells (e.g., fibroblasts). In some embodiments, the engineered tissue construct includes a population of stromal cells (e.g., fibroblasts) in an amount of from about 7×10⁸ to about 1.8×10¹² stromal cells (e.g., fibroblasts). In some embodiments, the engineered tissue construct includes a population of stromal cells (e.g., fibroblasts) in an amount of from about 8×10⁸ to about 1.8×10¹² stromal cells (e.g., fibroblasts). In some embodiments, the engineered tissue construct includes a population of stromal cells (e.g., fibroblasts) in an amount of from about 9×10⁸ to about 1.8×10¹² stromal cells (e.g., fibroblasts). In some embodiments, the engineered tissue construct includes a population of stromal cells (e.g., fibroblasts) in an amount of from about 1×10⁹ to about 1.8×10¹² stromal cells (e.g., fibroblasts). In some embodiments, the engineered tissue construct includes a population of stromal cells (e.g., fibroblasts) in an amount of from about 2×10⁹ to about 1.8×10¹² stromal cells (e.g., fibroblasts). In some embodiments, the engineered tissue construct includes a population of stromal cells (e.g., fibroblasts) in an amount of from about 3×10⁹ to about 1.8×10¹² stromal cells (e.g., fibroblasts). In some embodiments, the engineered tissue construct includes a population of stromal cells (e.g., fibroblasts) in an amount of from about 4×10⁹ to about 1.8×10¹² stromal cells (e.g., fibroblasts). In some embodiments, the engineered tissue construct includes a population of stromal cells (e.g., fibroblasts) in an amount of from about 5×10⁹ to about 1.8×10¹² stromal cells (e.g., fibroblasts). In some embodiments, the engineered tissue construct includes a population of stromal cells (e.g., fibroblasts) in an amount of from about 6×10⁹ to about 1.8×10¹² stromal cells (e.g., fibroblasts). In some embodiments, the engineered tissue construct includes a population of stromal cells (e.g., fibroblasts) in an amount of from about 7×10⁹ to about 1.8×10¹² stromal cells (e.g., fibroblasts). In some embodiments, the engineered tissue construct includes a population of stromal cells (e.g., fibroblasts) in an amount of from about 8×10⁹ to about 1.8×10¹² stromal cells (e.g., fibroblasts). In some embodiments, the engineered tissue construct includes a population of stromal cells (e.g., fibroblasts) in an amount of from about 9×10⁹ to about 1.8×10¹² stromal cells (e.g., fibroblasts). In some embodiments, the engineered tissue construct includes a population of stromal cells (e.g., fibroblasts) in an amount of from about 1×10¹⁰ to about 1.8×10¹² stromal cells (e.g., fibroblasts). In some embodiments, the engineered tissue construct includes a population of stromal cells (e.g., fibroblasts) in an amount of from about 2×10¹⁰ to about 1.8×10¹² stromal cells (e.g., fibroblasts). In some embodiments, the engineered tissue construct includes a population of stromal cells (e.g., fibroblasts) in an amount of from about 3×10¹⁰ to about 1.8×10¹² stromal cells (e.g., fibroblasts). In some embodiments, the engineered tissue construct includes a population of stromal cells (e.g., fibroblasts) in an amount of from about 4×10¹⁰ to about 1.8×10¹² stromal cells (e.g., fibroblasts). In some embodiments, the engineered tissue construct includes a population of stromal cells (e.g., fibroblasts) in an amount of from about 5×10¹⁰ to about 1.8×10¹² stromal cells (e.g., fibroblasts). In some embodiments, the engineered tissue construct includes a population of stromal cells (e.g., fibroblasts) in an amount of from about 6×10¹⁰ to about 1.8×10¹² stromal cells (e.g., fibroblasts). In some embodiments, the engineered tissue construct includes a population of stromal cells (e.g., fibroblasts) in an amount of from about 7×10¹⁰ to about 1.8×10¹² stromal cells (e.g., fibroblasts). In some embodiments, the engineered tissue construct includes a population of stromal cells (e.g., fibroblasts) in an amount of from about 8×10¹⁰ to about 1.8×10¹² stromal cells (e.g., fibroblasts). In some embodiments, the engineered tissue construct includes a population of stromal cells (e.g., fibroblasts) in an amount of from about 9×10¹⁰ to about 1.8×10¹² stromal cells (e.g., fibroblasts). In some embodiments, the engineered tissue construct includes a population of stromal cells (e.g., fibroblasts) in an amount of from about 1×10¹¹ to about 1.8×10¹² stromal cells (e.g., fibroblasts). In some embodiments, the engineered tissue construct includes a population of stromal cells (e.g., fibroblasts) in an amount of from about 2×10¹¹ to about 1.8×10¹² stromal cells (e.g., fibroblasts). In some embodiments, the engineered tissue construct includes a population of stromal cells (e.g., fibroblasts) in an amount of from about 3×10¹¹ to about 1.8×10¹² stromal cells (e.g., fibroblasts). In some embodiments, the engineered tissue construct includes a population of stromal cells (e.g., fibroblasts) in an amount of from about 4×10¹¹ to about 1.8×10¹² stromal cells (e.g., fibroblasts). In some embodiments, the engineered tissue construct includes a population of stromal cells (e.g., fibroblasts) in an amount of from about 5×10¹¹ to about 1.8×10¹² stromal cells (e.g., fibroblasts). In some embodiments, the engineered tissue construct includes a population of stromal cells (e.g., fibroblasts) in an amount of from about 6×10¹¹ to about 1.8×10¹² stromal cells (e.g., fibroblasts). In some embodiments, the engineered tissue construct includes a population of stromal cells (e.g., fibroblasts) in an amount of from about 7×10¹¹ to about 1.8×10¹² stromal cells (e.g., fibroblasts). In some embodiments, the engineered tissue construct includes a population of stromal cells (e.g., fibroblasts) in an amount of from about 8×10¹¹ to about 1.8×10¹² stromal cells (e.g., fibroblasts). In some embodiments, the engineered tissue construct includes a population of stromal cells (e.g., fibroblasts) in an amount of from about 9×10¹¹ to about 1.8×10¹² stromal cells (e.g., fibroblasts). In some embodiments, the engineered tissue construct includes a population of stromal cells (e.g., fibroblasts) in an amount of from about 1×10¹² to about 1.8×10¹² stromal cells (e.g., fibroblasts).

In some embodiments, the engineered tissue construct includes 0 stromal cells (e.g., fibroblasts). In some embodiments, the engineered tissue construct includes a population of stromal cells (e.g., fibroblasts) in an amount of about 1 stromal cell (e.g., fibroblast). In some embodiments, the engineered tissue construct includes a population of stromal cells (e.g., fibroblasts) in an amount of about 10 stromal cells (e.g., fibroblasts). In some embodiments, the engineered tissue construct includes a population of stromal cells (e.g., fibroblasts) in an amount of about 100 stromal cells (e.g., fibroblasts). In some embodiments, the engineered tissue construct includes a population of stromal cells (e.g., fibroblasts) in an amount of about 1×10³ stromal cells (e.g., fibroblasts). In some embodiments, the engineered tissue construct includes a population of stromal cells (e.g., fibroblasts) in an amount about 2×10³ stromal cells (e.g., fibroblasts). In some embodiments, the engineered tissue construct includes a population of stromal cells (e.g., fibroblasts) in an amount of about 3×10³ stromal cells (e.g., fibroblasts). In some embodiments, the engineered tissue construct includes a population of stromal cells (e.g., fibroblasts) in an amount of about 4×10³ stromal cells (e.g., fibroblasts). In some embodiments, the engineered tissue construct includes a population of stromal cells (e.g., fibroblasts) in an amount of about 5×10³ stromal cells (e.g., fibroblasts). In some embodiments, the engineered tissue construct includes a population of stromal cells (e.g., fibroblasts) in an amount of about 6×10³ stromal cells (e.g., fibroblasts). In some embodiments, the engineered tissue construct includes a population of stromal cells (e.g., fibroblasts) in an amount of about 7×10³ stromal cells (e.g., fibroblasts). In some embodiments, the engineered tissue construct includes a population of stromal cells (e.g., fibroblasts) in an amount of about 8×10³ stromal cells (e.g., fibroblasts). In some embodiments, the engineered tissue construct includes a population of stromal cells (e.g., fibroblasts) in an amount of about 9×10³ stromal cells (e.g., fibroblasts). In some embodiments, the engineered tissue construct includes a population of stromal cells (e.g., fibroblasts) in an amount of about 1×10⁴ stromal cells (e.g., fibroblasts). In some embodiments, the engineered tissue construct includes a population of stromal cells (e.g., fibroblasts) in an amount of about 2×10⁴ stromal cells (e.g., fibroblasts). In some embodiments, the engineered tissue construct includes a population of stromal cells (e.g., fibroblasts) in an amount of about 3×10⁴ stromal cells (e.g., fibroblasts). In some embodiments, the engineered tissue construct includes a population of stromal cells (e.g., fibroblasts) in an amount of about 4×10⁴ stromal cells (e.g., fibroblasts). In some embodiments, the engineered tissue construct includes a population of stromal cells (e.g., fibroblasts) in an amount of about 5×10⁴ stromal cells (e.g., fibroblasts). In some embodiments, the engineered tissue construct includes a population of stromal cells (e.g., fibroblasts) in an amount of about 6×10⁴ stromal cells (e.g., fibroblasts). In some embodiments, the engineered tissue construct includes a population of stromal cells (e.g., fibroblasts) in an amount of about 7×10⁴ stromal cells (e.g., fibroblasts). In some embodiments, the engineered tissue construct includes a population of stromal cells (e.g., fibroblasts) in an amount of about 8×10⁴ stromal cells (e.g., fibroblasts). In some embodiments, the engineered tissue construct includes a population of stromal cells (e.g., fibroblasts) in an amount of about 9×10⁴ stromal cells (e.g., fibroblasts). In some embodiments, the engineered tissue construct includes a population of stromal cells (e.g., fibroblasts) in an amount of about 1×10⁵ stromal cells (e.g., fibroblasts). In some embodiments, the engineered tissue construct includes a population of stromal cells (e.g., fibroblasts) in an amount of about 2×10⁵ stromal cells (e.g., fibroblasts). In some embodiments, the engineered tissue construct includes a population of stromal cells (e.g., fibroblasts) in an amount of about 3×10⁵ stromal cells (e.g., fibroblasts). In some embodiments, the engineered tissue construct includes a population of stromal cells (e.g., fibroblasts) in an amount of about 4×10⁵ stromal cells (e.g., fibroblasts). In some embodiments, the engineered tissue construct includes a population of stromal cells (e.g., fibroblasts) in an amount of about 5×10⁵ stromal cells (e.g., fibroblasts). In some embodiments, the engineered tissue construct includes a population of stromal cells (e.g., fibroblasts) in an amount of about 6×10⁵ stromal cells (e.g., fibroblasts). In some embodiments, the engineered tissue construct includes a population of stromal cells (e.g., fibroblasts) in an amount of about 7×10⁵ stromal cells (e.g., fibroblasts). In some embodiments, the engineered tissue construct includes a population of stromal cells (e.g., fibroblasts) in an amount of about 8×10⁵ stromal cells (e.g., fibroblasts). In some embodiments, the engineered tissue construct includes a population of stromal cells (e.g., fibroblasts) in an amount of about 9×10⁵ stromal cells (e.g., fibroblasts). In some embodiments, the engineered tissue construct includes a population of stromal cells (e.g., fibroblasts) in an amount of about 1×10⁶ stromal cells (e.g., fibroblasts). In some embodiments, the engineered tissue construct includes a population of stromal cells (e.g., fibroblasts) in an amount of about 2×10⁶ stromal cells (e.g., fibroblasts). In some embodiments, the engineered tissue construct includes a population of stromal cells (e.g., fibroblasts) in an amount of about 3×10⁶ stromal cells (e.g., fibroblasts). In some embodiments, the engineered tissue construct includes a population of stromal cells (e.g., fibroblasts) in an amount of about 4×10⁶ stromal cells (e.g., fibroblasts). In some embodiments, the engineered tissue construct includes a population of stromal cells (e.g., fibroblasts) in an amount of about 5×10⁶ stromal cells (e.g., fibroblasts). In some embodiments, the engineered tissue construct includes a population of stromal cells (e.g., fibroblasts) in an amount of about 6×10⁶ stromal cells (e.g., fibroblasts). In some embodiments, the engineered tissue construct includes a population of stromal cells (e.g., fibroblasts) in an amount of about 7×10⁶ stromal cells (e.g., fibroblasts). In some embodiments, the engineered tissue construct includes a population of stromal cells (e.g., fibroblasts) in an amount of about 8×10⁶ stromal cells (e.g., fibroblasts). In some embodiments, the engineered tissue construct includes a population of stromal cells (e.g., fibroblasts) in an amount of about 9×10⁶ stromal cells (e.g., fibroblasts). In some embodiments, the engineered tissue construct includes a population of stromal cells (e.g., fibroblasts) in an amount of about 1×10⁷ stromal cells (e.g., fibroblasts). In some embodiments, the engineered tissue construct includes a population of stromal cells (e.g., fibroblasts) in an amount of about 2×10⁷ stromal cells (e.g., fibroblasts). In some embodiments, the engineered tissue construct includes a population of stromal cells (e.g., fibroblasts) in an amount of about 3×10⁷ stromal cells (e.g., fibroblasts). In some embodiments, the engineered tissue construct includes a population of stromal cells (e.g., fibroblasts) in an amount of about 4×10⁷ stromal cells (e.g., fibroblasts). In some embodiments, the engineered tissue construct includes a population of stromal cells (e.g., fibroblasts) in an amount of about 5×10⁷ stromal cells (e.g., fibroblasts). In some embodiments, the engineered tissue construct includes a population of stromal cells (e.g., fibroblasts) in an amount of about 6×10⁷ stromal cells (e.g., fibroblasts). In some embodiments, the engineered tissue construct includes a population of stromal cells (e.g., fibroblasts) in an amount of about 7×10⁷ stromal cells (e.g., fibroblasts). In some embodiments, the engineered tissue construct includes a population of stromal cells (e.g., fibroblasts) in an amount of about 8×10⁷ stromal cells (e.g., fibroblasts). In some embodiments, the engineered tissue construct includes a population of stromal cells (e.g., fibroblasts) in an amount of about 9×10⁷ stromal cells (e.g., fibroblasts). In some embodiments, the engineered tissue construct includes a population of stromal cells (e.g., fibroblasts) in an amount of about 1×10⁸ stromal cells (e.g., fibroblasts). In some embodiments, the engineered tissue construct includes a population of stromal cells (e.g., fibroblasts) in an amount of about 2×10⁸ stromal cells (e.g., fibroblasts). In some embodiments, the engineered tissue construct includes a population of stromal cells (e.g., fibroblasts) in an amount of about 3×10⁸ stromal cells (e.g., fibroblasts). In some embodiments, the engineered tissue construct includes a population of stromal cells (e.g., fibroblasts) in an amount of about 4×10⁸ stromal cells (e.g., fibroblasts). In some embodiments, the engineered tissue construct includes a population of stromal cells (e.g., fibroblasts) in an amount of about 5×10⁸ stromal cells (e.g., fibroblasts). In some embodiments, the engineered tissue construct includes a population of stromal cells (e.g., fibroblasts) in an amount of about 6×10⁸ stromal cells (e.g., fibroblasts). In some embodiments, the engineered tissue construct includes a population of stromal cells (e.g., fibroblasts) in an amount of about 7×10⁸ stromal cells (e.g., fibroblasts). In some embodiments, the engineered tissue construct includes a population of stromal cells (e.g., fibroblasts) in an amount of about 8×10⁸ stromal cells (e.g., fibroblasts). In some embodiments, the engineered tissue construct includes a population of stromal cells (e.g., fibroblasts) in an amount of about 9×10⁸ stromal cells (e.g., fibroblasts). In some embodiments, the engineered tissue construct includes a population of stromal cells (e.g., fibroblasts) in an amount of about 1×10⁹ stromal cells (e.g., fibroblasts). In some embodiments, the engineered tissue construct includes a population of stromal cells (e.g., fibroblasts) in an amount of about 2×10⁹ stromal cells (e.g., fibroblasts). In some embodiments, the engineered tissue construct includes a population of stromal cells (e.g., fibroblasts) in an amount of about 3×10⁹ stromal cells (e.g., fibroblasts). In some embodiments, the engineered tissue construct includes a population of stromal cells (e.g., fibroblasts) in an amount of about 4×10⁹ stromal cells (e.g., fibroblasts). In some embodiments, the engineered tissue construct includes a population of stromal cells (e.g., fibroblasts) in an amount of about 5×10⁹ stromal cells (e.g., fibroblasts). In some embodiments, the engineered tissue construct includes a population of stromal cells (e.g., fibroblasts) in an amount of about 6×10⁹ stromal cells (e.g., fibroblasts). In some embodiments, the engineered tissue construct includes a population of stromal cells (e.g., fibroblasts) in an amount of about 7×10⁹ stromal cells (e.g., fibroblasts). In some embodiments, the engineered tissue construct includes a population of stromal cells (e.g., fibroblasts) in an amount of about 8×10⁹ stromal cells (e.g., fibroblasts). In some embodiments, the engineered tissue construct includes a population of stromal cells (e.g., fibroblasts) in an amount of about 9×10⁹ stromal cells (e.g., fibroblasts). In some embodiments, the engineered tissue construct includes a population of stromal cells (e.g., fibroblasts) in an amount of about 1×10¹⁰ stromal cells (e.g., fibroblasts). In some embodiments, the engineered tissue construct includes a population of stromal cells (e.g., fibroblasts) in an amount of about 2×10¹⁰ stromal cells (e.g., fibroblasts). In some embodiments, the engineered tissue construct includes a population of stromal cells (e.g., fibroblasts) in an amount of about 3×10¹⁰ stromal cells (e.g., fibroblasts). In some embodiments, the engineered tissue construct includes a population of stromal cells (e.g., fibroblasts) in an amount of about 4×10¹⁰ stromal cells (e.g., fibroblasts). In some embodiments, the engineered tissue construct includes a population of stromal cells (e.g., fibroblasts) in an amount of about 5×10¹⁰ stromal cells (e.g., fibroblasts). In some embodiments, the engineered tissue construct includes a population of stromal cells (e.g., fibroblasts) in an amount of about 6×10¹⁰ stromal cells (e.g., fibroblasts). In some embodiments, the engineered tissue construct includes a population of stromal cells (e.g., fibroblasts) in an amount of about 7×10¹⁰ stromal cells (e.g., fibroblasts). In some embodiments, the engineered tissue construct includes a population of stromal cells (e.g., fibroblasts) in an amount of about 8×10¹⁰ stromal cells (e.g., fibroblasts). In some embodiments, the engineered tissue construct includes a population of stromal cells (e.g., fibroblasts) in an amount of about 9×10¹⁰ stromal cells (e.g., fibroblasts). In some embodiments, the engineered tissue construct includes a population of stromal cells (e.g., fibroblasts) in an amount of about 1×10¹¹ stromal cells (e.g., fibroblasts). In some embodiments, the engineered tissue construct includes a population of stromal cells (e.g., fibroblasts) in an amount of about 2×10¹¹ stromal cells (e.g., fibroblasts). In some embodiments, the engineered tissue construct includes a population of stromal cells (e.g., fibroblasts) in an amount of about 3×10¹¹ stromal cells (e.g., fibroblasts). In some embodiments, the engineered tissue construct includes a population of stromal cells (e.g., fibroblasts) in an amount of about 4×10¹¹ stromal cells (e.g., fibroblasts). In some embodiments, the engineered tissue construct includes a population of stromal cells (e.g., fibroblasts) in an amount of about 5×10¹¹ stromal cells (e.g., fibroblasts). In some embodiments, the engineered tissue construct includes a population of stromal cells (e.g., fibroblasts) in an amount of about 6×10¹¹ stromal cells (e.g., fibroblasts). In some embodiments, the engineered tissue construct includes a population of stromal cells (e.g., fibroblasts) in an amount of about 7×10¹¹ stromal cells (e.g., fibroblasts). In some embodiments, the engineered tissue construct includes a population of stromal cells (e.g., fibroblasts) in an amount of about 8×10¹¹ stromal cells (e.g., fibroblasts). In some embodiments, the engineered tissue construct includes a population of stromal cells (e.g., fibroblasts) in an amount of about 9×10¹¹ stromal cells (e.g., fibroblasts). In some embodiments, the engineered tissue construct includes a population of stromal cells (e.g., fibroblasts) in an amount of about 1×10¹² stromal cells (e.g., fibroblasts). In some embodiments, the engineered tissue construct includes a population of stromal cells (e.g., fibroblasts) in an amount of about 1.8×10¹² stromal cells (e.g., fibroblasts).

In some embodiments, the stromal cells are growth-arrested stromal cells.

Combination of Hepatocytes and Stromal cells

The cellular compositions disclosed herein can be provided as a suspension (e.g., in a biocompatible scaffold) containing the hepatocytes and optionally the stromal cells (e.g., fibroblasts). In some embodiments, the population of hepatocytes and the optional population of stromal cells are aggregated in spheroids. For example, in some embodiments, the population of hepatocytes and the optional population of stromal cells are aggregated in spheroids and the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

In some embodiments, the hepatocytes and optional stromal cells are in a biocompatible scaffold, and the population of hepatocytes and the population of stromal cells together account for at least 70% (e.g., at least 71%, 72%, 73%, 74%, 75%, 80%, 85%, 90%, 95%, or 100%) of the total cells in the engineered tissue construct. For example, in some embodiments, the population of hepatocytes and the population of stromal cells together account for at least 71% of the total cells in the engineered tissue construct. In some embodiments, the population of hepatocytes and the population of stromal cells together account for at least 72% of the total cells in the engineered tissue construct. In some embodiments, the population of hepatocytes and the population of stromal cells together account for at least 73% of the total cells in the engineered tissue construct. In some embodiments, the population of hepatocytes and the population of stromal cells together account for at least 74% of the total cells in the engineered tissue construct. In some embodiments, the population of hepatocytes and the population of stromal cells together account for at least 75% of the total cells in the engineered tissue construct. In some embodiments, the population of hepatocytes and the population of stromal cells together account for at least 80% of the total cells in the engineered tissue construct. In some embodiments, the population of hepatocytes and the population of stromal cells together account for at least 85% of the total cells in the engineered tissue construct. In some embodiments, the population of hepatocytes and the population of stromal cells together account for at least 90% of the total cells in the engineered tissue construct. In some embodiments, the population of hepatocytes and the population of stromal cells together account for at least 95% of the total cells in the engineered tissue construct. In some embodiments, the population of hepatocytes and the population of stromal cells together account for 100% of the total cells in the engineered tissue construct.

In some embodiments, the hepatocytes and optional stromal cells are distributed non-homogenously along the z-axis of the biocompatible scaffold.

In some embodiments, the hepatocytes and optional stromal cells are distributed homogenously along the x-axis of the biocompatible scaffold.

In some embodiments, the hepatocytes and optional stromal cells are distributed homogenously along the 7-axis of the biocompatible scaffold.

In some embodiments, the ratio of hepatocytes to stromal cells (e.g., fibroblasts) is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

For example, in some embodiments, the ratio of hepatocytes to stromal cells (e.g., fibroblasts) is between 1:9 and 4:1. In some embodiments, the ratio of hepatocytes to stromal cells (e.g., fibroblasts) is between 1:8 and 4:1. In some embodiments, the ratio of hepatocytes to stromal cells (e.g., fibroblasts) is between 1:7 and 4:1. In some embodiments, the ratio of hepatocytes to stromal cells (e.g., fibroblasts) is between 1:6 and 4:1. In some embodiments, the ratio of hepatocytes to stromal cells (e.g., fibroblasts) is between 1:5 and 4:1. In some embodiments, the ratio of hepatocytes to stromal cells (e.g., fibroblasts) is between 1:4 and 4:1. In some embodiments, the ratio of hepatocytes to stromal cells (e.g., fibroblasts) is between 1:3 and 4:1. In some embodiments, the ratio of hepatocytes to stromal cells (e.g., fibroblasts) is between 1:2 and 4:1. In some embodiments, the ratio of hepatocytes to stromal cells (e.g., fibroblasts) is between 1:1 and 4:1. In some embodiments, the ratio of hepatocytes to stromal cells (e.g., fibroblasts) is between 1:0 and 4:1.

In some embodiments, the ratio of hepatocytes to stromal cells (e.g., fibroblasts) is between 1:10 and 3:1. In some embodiments, the ratio of hepatocytes to stromal cells (e.g., fibroblasts) is between 1:9 and 3:1. In some embodiments, the ratio of hepatocytes to stromal cells (e.g., fibroblasts) is between 1:8 and 3:1. In some embodiments, the ratio of hepatocytes to stromal cells (e.g., fibroblasts) is between 1:7 and 3:1. In some embodiments, the ratio of hepatocytes to stromal cells (e.g., fibroblasts) is between 1:6 and 3:1. In some embodiments, the ratio of hepatocytes to stromal cells (e.g., fibroblasts) is between 1:5 and 3:1. In some embodiments, the ratio of hepatocytes to stromal cells (e.g., fibroblasts) is between 1:4 and 3:1. In some embodiments, the ratio of hepatocytes to stromal cells (e.g., fibroblasts) is between 1:3 and 3:1. In some embodiments, the ratio of hepatocytes to stromal cells (e.g., fibroblasts) is between 1:2 and 3:1. In some embodiments, the ratio of hepatocytes to stromal cells (e.g., fibroblasts) is between 1:1 and 3:1. In some embodiments, the ratio of hepatocytes to stromal cells (e.g., fibroblasts) is between 1:0 and 3:1.

In some embodiments, the ratio of hepatocytes to stromal cells (e.g., fibroblasts) is between 1:10 and 2:1. In some embodiments, the ratio of hepatocytes to stromal cells (e.g., fibroblasts) is between 1:9 and 2:1. In some embodiments, the ratio of hepatocytes to stromal cells (e.g., fibroblasts) is between 1:8 and 2:1. In some embodiments, the ratio of hepatocytes to stromal cells (e.g., fibroblasts) is between 1:7 and 2:1. In some embodiments, the ratio of hepatocytes to stromal cells (e.g., fibroblasts) is between 1:6 and 2:1. In some embodiments, the ratio of hepatocytes to stromal cells (e.g., fibroblasts) is between 1:5 and 2:1. In some embodiments, the ratio of hepatocytes to stromal cells (e.g., fibroblasts) is between 1:4 and 2:1. In some embodiments, the ratio of hepatocytes to stromal cells (e.g., fibroblasts) is between 1:3 and 2:1. In some embodiments, the ratio of hepatocytes to stromal cells (e.g., fibroblasts) is between 1:2 and 2:1. In some embodiments, the ratio of hepatocytes to stromal cells (e.g., fibroblasts) is between 1:1 and 2:1. In some embodiments, the ratio of hepatocytes to stromal cells (e.g., fibroblasts) is between 1:0 and 2:1.

In some embodiments, the ratio of hepatocytes to stromal cells (e.g., fibroblasts) is between 1:10 and 1:1. In some embodiments, the ratio of hepatocytes to stromal cells (e.g., fibroblasts) is between 1:9 and 1:1. In some embodiments, the ratio of hepatocytes to stromal cells (e.g., fibroblasts) is between 1:8 and 1:1. In some embodiments, the ratio of hepatocytes to stromal cells (e.g., fibroblasts) is between 1:7 and 1:1. In some embodiments, the ratio of hepatocytes to stromal cells (e.g., fibroblasts) is between 1:6 and 1:1. In some embodiments, the ratio of hepatocytes to stromal cells (e.g., fibroblasts) is between 1:5 and 1:1. In some embodiments, the ratio of hepatocytes to stromal cells (e.g., fibroblasts) is between 1:4 and 1:1. In some embodiments, the ratio of hepatocytes to stromal cells (e.g., fibroblasts) is between 1:3 and 1:1. In some embodiments, the ratio of hepatocytes to stromal cells (e.g., fibroblasts) is between 1:2 and 1:1. In some embodiments, the ratio of hepatocytes to stromal cells (e.g., fibroblasts) is between 1:1 and 1:1. In some embodiments, the ratio of hepatocytes to stromal cells (e.g., fibroblasts) is between 1:0 and 1:1.

In some embodiments, the ratio of hepatocytes to stromal cells (e.g., fibroblasts) is between 1:10 and 1:0. In some embodiments, the ratio of hepatocytes to stromal cells (e.g., fibroblasts) is between 1:9 and 1:0. In some embodiments, the ratio of hepatocytes to stromal cells (e.g., fibroblasts) is between 1:8 and 1:0. In some embodiments, the ratio of hepatocytes to stromal cells (e.g., fibroblasts) is between 1:7 and 1:0. In some embodiments, the ratio of hepatocytes to stromal cells (e.g., fibroblasts) is between 1:6 and 1:0. In some embodiments, the ratio of hepatocytes to stromal cells (e.g., fibroblasts) is between 1:5 and 1:0. In some embodiments, the ratio of hepatocytes to stromal cells (e.g., fibroblasts) is between 1:4 and 1:0. In some embodiments, the ratio of hepatocytes to stromal cells (e.g., fibroblasts) is between 1:3 and 1:0. In some embodiments, the ratio of hepatocytes to stromal cells (e.g., fibroblasts) is between 1:2 and 1:0. In some embodiments, the ratio of hepatocytes to stromal cells (e.g., fibroblasts) is between 1:1 and 1:0.

In some embodiments, the layer of hepatocyte and optional stromal cell aggregates in the biocompatible scaffold is from 100 μm to 2 mm (e.g., 200 μm to 1900 μm, 300 μm to 1800 μm, 400 μm to 1700 μm, 500 μm to 1600 μm, 600 μm to 1500 μm, 700 μm to 1400 μm, 800 μm to 1300 μm, 900 μm to 1200 μm, or 1000 μm to 1100 μm) thick. For example, in some embodiments, the layer of hepatocyte and optional stromal cell aggregates in the biocompatible scaffold is from 200 μm to 1900 μm thick. In some embodiments, the layer of hepatocyte and optional stromal cell aggregates in the biocompatible scaffold is from 300 μm to 1800 μm thick. In some embodiments, the layer of hepatocyte and optional stromal cell aggregates in the biocompatible scaffold is from 400 μm to 1700 μm thick. In some embodiments, the layer of hepatocyte and optional stromal cell aggregates in the biocompatible scaffold is from 500 μm to 1600 μm thick. In some embodiments, the layer of hepatocyte and optional stromal cell aggregates in the biocompatible scaffold is from 600 μm to 1500 μm thick. In some embodiments, the layer of hepatocyte and optional stromal cell aggregates in the biocompatible scaffold is from 700 μm to 1400 μm thick. In some embodiments, the layer of hepatocyte and optional stromal cell aggregates in the biocompatible scaffold is from 800 μm to 1300 μm thick. In some embodiments, the layer of hepatocyte and optional stromal cell aggregates in the biocompatible scaffold is from 900 μm to 1200 μm thick. In some embodiments, the layer of hepatocyte and optional stromal cell aggregates in the biocompatible scaffold is from 1000 μm to 1100 μm thick. For example, in some embodiments, the layer of hepatocyte and optional stromal cell aggregates in the biocompatible scaffold is from 100 μm to 1 mm (e.g., 200 μm to 900 μm, 300 μm to 800 μm, 400 μm to 700 μm, or 500 μm to 600 μm) thick. For example, in some embodiments, the layer of hepatocyte and stromal cell aggregates in the biocompatible scaffold is from 200 μm to 900 μm thick. In some embodiments, the layer of hepatocyte and stromal cell aggregates in the biocompatible scaffold is from 300 μm to 800 μm thick. In some embodiments, the layer of hepatocyte and stromal cell aggregates in the biocompatible scaffold is from 300 μm to 500 μm thick. In some embodiments, the layer of hepatocyte and stromal cell aggregates in the biocompatible scaffold is from 400 μm to 700 μm thick. In some embodiments, the layer of hepatocyte and stromal cell aggregates in the biocompatible scaffold is from 500 μm to 600 μm thick.

In some embodiments, the layer of hepatocyte and stromal cell aggregates in the biocompatible scaffold is 100 μm thick. In some embodiments, the layer of hepatocyte and stromal cell aggregates in the biocompatible scaffold is 200 μm thick. In some embodiments, the layer of hepatocyte and stromal cell aggregates in the biocompatible scaffold is 300 μm thick. In some embodiments, the layer of hepatocyte and stromal cell aggregates in the biocompatible scaffold is 400 μm thick. In some embodiments, the layer of hepatocyte and stromal cell aggregates in the biocompatible scaffold is 500 μm thick. In some embodiments, the layer of hepatocyte and stromal cell aggregates in the biocompatible scaffold is 600 μm thick. In some embodiments, the layer of hepatocyte and stromal cell aggregates in the biocompatible scaffold is 700 μm thick. In some embodiments, the layer of hepatocyte and stromal cell aggregates in the biocompatible scaffold is 800 μm thick. In some embodiments, the layer of hepatocyte and stromal cell aggregates in the biocompatible scaffold is 900 μm thick. In some embodiments, the layer of hepatocyte and stromal cell aggregates in the biocompatible scaffold is 1 mm thick. In some embodiments, the layer of hepatocyte and stromal cell aggregates in the biocompatible scaffold is 1.5 mm thick. In some embodiments, the layer of hepatocyte and stromal cell aggregates in the biocompatible scaffold is 2 mm thick.

In some embodiments, the density of hepatocytes in the layer of hepatocyte and optional stromal cell aggregates in the biocompatible scaffold is from 0.06 M/cm² to 150 M/cm² (e.g., 0.07 M/cm² to 149 M/cm², 0.08 M/cm² to 148 M/cm², 0.09 M/cm² to 147 M/cm², 0.1 M/cm² to 146 M/cm², 0.2 M/cm² to 145 M/cm², 0.3 M/cm² to 140 M/cm², 0.4 M/cm² to 130 M/cm², 0.5 M/cm² to 120 M/cm², 1 M/cm² to 110 M/cm², 2 M/cm² to 100 M/cm², 3 M/cm² to 50 M/cm², 4 M/cm² to 40 M/cm², 5 M/cm² to 30 M/cm², or 10 M/cm² to 20 M/cm²). For example, in some embodiments, the density of hepatocytes in the layer of hepatocyte and stromal cell aggregates in the biocompatible scaffold is from 0.07 M/cm² to 149 M/cm². In some embodiments, the density of hepatocytes in the layer of hepatocyte and stromal cell aggregates in the biocompatible scaffold is from 0.08 M/cm² to 148 M/cm². In some embodiments, the density of hepatocytes in the layer of hepatocyte and stromal cell aggregates in the biocompatible scaffold is from 0.09 M/cm² to 147 M/cm². In some embodiments, the density of hepatocytes in the layer of hepatocyte and stromal cell aggregates in the biocompatible scaffold is from 0.1 M/cm² to 146 M/cm². In some embodiments, the density of hepatocytes in the layer of hepatocyte and stromal cell aggregates in the biocompatible scaffold is from 0.2 M/cm² to 145 M/cm². In some embodiments, the density of hepatocytes in the layer of hepatocyte and stromal cell aggregates in the biocompatible scaffold is from 0.3 M/cm² to 140 M/cm². In some embodiments, the density of hepatocytes in the layer of hepatocyte and stromal cell aggregates in the biocompatible scaffold is from 0.4 M/cm² to 130 M/cm². In some embodiments, the density of hepatocytes in the layer of hepatocyte and stromal cell aggregates in the biocompatible scaffold is from 0.5 M/cm² to 120 M/cm². In some embodiments, the density of hepatocytes in the layer of hepatocyte and stromal cell aggregates in the biocompatible scaffold is from 1 M/cm² to 110 M/cm². In some embodiments, the density of hepatocytes in the layer of hepatocyte and stromal cell aggregates in the biocompatible scaffold is from 2 M/cm² to 100 M/cm². In some embodiments, the density of hepatocytes in the layer of hepatocyte and stromal cell aggregates in the biocompatible scaffold is from 3 M/cm² to 50 M/cm². In some embodiments, the density of hepatocytes in the layer of hepatocyte and stromal cell aggregates in the biocompatible scaffold is from 4 M/cm² to 40 M/cm². In some embodiments, the density of hepatocytes in the layer of hepatocyte and stromal cell aggregates in the biocompatible scaffold is from 5 M/cm² to 30 M/cm². In some embodiments, the density of hepatocytes in the layer of hepatocyte and stromal cell aggregates in the biocompatible scaffold is from 10 M/cm² to 20 M/cm². In some embodiments, the density of hepatocytes in the layer of hepatocyte and stromal cell aggregates in the biocompatible scaffold is 0.06 M/cm². In some embodiments, the density of hepatocytes in the layer of hepatocyte and stromal cell aggregates in the biocompatible scaffold is 0.07 M/cm². In some embodiments, the density of hepatocytes in the layer of hepatocyte and stromal cell aggregates in the biocompatible scaffold is 0.08 M/cm². In some embodiments, the density of hepatocytes in the layer of hepatocyte and stromal cell aggregates in the biocompatible scaffold is 0.09 M/cm². In some embodiments, the density of hepatocytes in the layer of hepatocyte and stromal cell aggregates in the biocompatible scaffold is 0.1 M/cm². In some embodiments, the density of hepatocytes in the layer of hepatocyte and stromal cell aggregates in the biocompatible scaffold is 0.2 M/cm². In some embodiments, the density of hepatocytes in the layer of hepatocyte and stromal cell aggregates in the biocompatible scaffold is 0.3 M/cm². In some embodiments, the density of hepatocytes in the layer of hepatocyte and stromal cell aggregates in the biocompatible scaffold is 0.4 M/cm². In some embodiments, the density of hepatocytes in the layer of hepatocyte and stromal cell aggregates in the biocompatible scaffold is 0.5 M/cm². In some embodiments, the density of hepatocytes in the layer of hepatocyte and stromal cell aggregates in the biocompatible scaffold is 1 M/cm². In some embodiments, the density of hepatocytes in the layer of hepatocyte and stromal cell aggregates in the biocompatible scaffold is 2 M/cm². In some embodiments, the density of hepatocytes in the layer of hepatocyte and stromal cell aggregates in the biocompatible scaffold is 3 M/cm². In some embodiments, the density of hepatocytes in the layer of hepatocyte and stromal cell aggregates in the biocompatible scaffold is 4 M/cm². In some embodiments, the density of hepatocytes in the layer of hepatocyte and stromal cell aggregates in the biocompatible scaffold is 5 M/cm². In some embodiments, the density of hepatocytes in the layer of hepatocyte and stromal cell aggregates in the biocompatible scaffold is 10 M/cm². In some embodiments, the density of hepatocytes in the layer of hepatocyte and stromal cell aggregates in the biocompatible scaffold is 20 M/cm². In some embodiments, the density of hepatocytes in the layer of hepatocyte and stromal cell aggregates in the biocompatible scaffold is 30 M/cm². In some embodiments, the density of hepatocytes in the layer of hepatocyte and stromal cell aggregates in the biocompatible scaffold is 40 M/cm². In some embodiments, the density of hepatocytes in the layer of hepatocyte and stromal cell aggregates in the biocompatible scaffold is 50 M/cm². In some embodiments, the density of hepatocytes in the layer of hepatocyte and stromal cell aggregates in the biocompatible scaffold is 100 M/cm². In some embodiments, the density of hepatocytes in the layer of hepatocyte and stromal cell aggregates in the biocompatible scaffold is 110 M/cm². In some embodiments, the density of hepatocytes in the layer of hepatocyte and stromal cell aggregates in the biocompatible scaffold is 120 M/cm². In some embodiments, the density of hepatocytes in the layer of hepatocyte and stromal cell aggregates in the biocompatible scaffold is 130 M/cm². In some embodiments, the density of hepatocytes in the layer of hepatocyte and stromal cell aggregates in the biocompatible scaffold is 140 M/cm². In some embodiments, the density of hepatocytes in the layer of hepatocyte and stromal cell aggregates in the biocompatible scaffold is 145 M/cm². In some embodiments, the density of hepatocytes in the layer of hepatocyte and stromal cell aggregates in the biocompatible scaffold is 146 M/cm². In some embodiments, the density of hepatocytes in the layer of hepatocyte and stromal cell aggregates in the biocompatible scaffold is 147 M/cm². In some embodiments, the density of hepatocytes in the layer of hepatocyte and stromal cell aggregates in the biocompatible scaffold is 148 M/cm². In some embodiments, the density of hepatocytes in the layer of hepatocyte and stromal cell aggregates in the biocompatible scaffold is 149 M/cm². In some embodiments, the density of hepatocytes in the layer of hepatocyte and stromal cell aggregates in the biocompatible scaffold is 150 M/cm².

In some embodiments, the thickness of the layer is dependent upon the hepatocyte-density.

Other Cell Types In some embodiments, the engineered tissue construct includes less than 30% (e.g., less than 29%, 28%, 27%, 26%, 25%, 20%, 15%, 10%, or 21%) of other cell types. For example, other cell types may include stellate cells, Kupffer cells, pit cells, cholangiocytes, portal fibroblasts, and liver sinusoidal endothelial cells. In some embodiments, the engineered tissue construct includes less than 30% (e.g., less than 29%, 28%, 27%, 26%, 25%, 20%, 15%, 10%, or 21%) of stellate cells. In some embodiments, the engineered tissue construct includes less than 30% (e.g., less than 29%, 28%, 27%, 26%, 25%, 20%, 15%, 10%, or 21%) of Kupffer cells. In some embodiments, the engineered tissue construct includes less than 30% (e.g., less than 29%, 28%, 27%, 26%, 25%, 20%, 15%, 10%, or 21%) of pit cells. In some embodiments, the engineered tissue construct includes less than 30% (e.g., less than 29%, 28%, 27%, 26%, 25%, 20%, 15%, 10%, or 21%) of bile duct cells. In some embodiments, the engineered tissue construct exclusively includes hepatocytes.

In some embodiments, the engineered tissue construct includes endothelial cells. In some embodiments, the engineered tissue construct includes up to 30% (e.g., up to 29%, 28%, 27%, 26%, 25%, 20%, 15%, 10%, or 1%) of endothelial cells. For example, in some embodiments, the engineered tissue construct includes up to 29% of endothelial cells. In some embodiments, the engineered tissue construct includes up to 28% of endothelial cells. In some embodiments, the engineered tissue construct includes up to 27% of endothelial cells. In some embodiments, the engineered tissue construct includes up to 26% of endothelial cells. In some embodiments, the engineered tissue construct includes up to 25% of endothelial cells. In some embodiments, the engineered tissue construct includes up to 20% of endothelial cells. In some embodiments, the engineered tissue construct includes up to 15% of endothelial cells. In some embodiments, the engineered tissue construct includes up to 10% of endothelial cells. In some embodiments, the engineered tissue construct includes up to 1% of endothelial cells.

### Biocompatible Hydrogel Scaffolds

The engineered tissue constructs and cellular compositions disclosed herein may include a biocompatible scaffold or matrix. The cellular compositions disclosed herein can be provided as a suspension in a biocompatible scaffold containing the hepatocytes and stromal cells. In some embodiments, the population of hepatocytes and the population of stromal cells are aggregated in spheroids. In some embodiments, the biocompatible scaffold has an x-axis, a y-axis, and a z-axis. For example, in some embodiments, the population of hepatocytes and the optional population of stromal cells are aggregated in spheroids and the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

The biocompatible scaffold may be liquid, gel, semi-solid, or solid at room temperature (e.g., 25° C.). The biocompatible scaffold may be biodegradable or non-biodegradable. In some embodiments, the scaffold is bioresorbable or bioreplaceable. Exemplary biocompatible scaffolds include polymers and hydrogels, including collagen, fibrinogen, fibrin, chitosan, MATRIGEL™, dextrans including chemically cross-linkable or photo-cross-linkable dextrans, processed tissue matrix such as submucosal tissue, PEG hydrogels (e.g., heparin-conjugated PEG hydrogels), poly(lactic-co-glycolic acid) (PLGA), hydroxyethyl methacrylate (HEMA), gelatin, alginate, agarose, polysaccharides, hyaluronic acid (HA), peptide-based self-assembling gels, thermo-responsive poly(NIPAAm). A number of biopolymers are known to those skilled in the art (Bryant and Anseth, J. Biomed. Mater. Res. (2002) 59(1):63-72; Mann et al., Biomaterials (2001) 22 (22): 3045-3051; Mann et al., Biomaterials (2001) 22 (5):439-444, and Peppas et al., Eur. J. Pharm. Biopharm. (2000) 50(1), 27-46; all incorporated by reference). In other embodiments, the biocompatible scaffold may contain a biopolymer having any of a number of growth factors, adhesion molecules, degradation sites or bioactive agents to enhance cell viability or for any of a number of other reasons. Such molecules are well known to those skilled in the art.

In some embodiments, the PEG hydrogel may be chemically cross-linkable and/or modified with bifunctional groups.

In certain embodiments, the biocompatible scaffold includes allogeneic components, autologous components, or both allogeneic components and autologous components. In certain embodiments, the biocompatible scaffold includes synthetic or semi-synthetic materials. In certain embodiments, the biocompatible scaffold includes a framework or support, such as a fibrin-derived scaffold.

In some embodiments, the biocompatible scaffold is fibrin.

Biocompatible hydrogel scaffolds suitable for use include any polymer that is gellable in situ, e.g., one that does not require chemicals or conditions (e.g., temperature or pH) that are not cytocompatible. This includes both stable and biodegradable biopolymers.

Polymers for use herein are preferably crosslinked, for example, ionically crosslinked. In certain embodiments, the methods and constructs described herein use polymers in which polymerization can be promoted photochemically (i.e., photo crosslinked), by exposure to an appropriate wavelength of light (i.e., photopolymerizable) or a polymer which is weakened or rendered soluble by light exposure or other stimulus. Although some of the polymers listed above are not inherently light sensitive (e.g., collagen, HA), they may be made light sensitive by the addition of acrylate or other photosensitive groups.

In certain embodiments, the method utilizes a photoinitiator. A photoinitiator is a molecule that is capable of promoting polymerization of hydrogels upon exposure to an appropriate wavelength of light as defined by the reactive groups on the molecule. In the context of the disclosure, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-1propanone) are all activated with UV light (365 nm). Other crosslinking agents activated by wavelengths of light that are cytocompatible (e.g., blue light) can also be used with the methods described herein.

In other embodiments, the method involves the use of polymers bearing non-photochemically polymerizable moieties. In certain embodiments, the non-photochemically polymerizable moieties are Michael acceptors. Non-limiting examples of such Michael acceptor moieties include α,β-unsaturated ketones, esters, amides, sulfones, sulfoxides, phosphonates. Additional non-limiting examples of Michael acceptors include quinines and vinyl pyridines. In some embodiments, the polymerization of Michael acceptors is promoted by a nucleophile. Suitable nucleophiles include, but are not limited to thiols, amines, alcohols, and molecules possessing thiol, amine, and alcohol moieties. In certain embodiments, the disclosure features use of thermally crosslinked polymers.

In some embodiments, the z-axis of the biocompatible scaffold is from 500 μm to 5 mm (e.g., 600 μm to 4 mm, 700 μm to 3 mm, 800 μm to 2 mm, or 900 μm to 1 mm). For example, in some embodiments, the z-axis of the biocompatible scaffold is from 600 μm to 4 mm. In some embodiments, the z-axis of the biocompatible scaffold is from 700 μm to 3 mm. In some embodiments, the z-axis of the biocompatible scaffold is from 800 μm to 2 mm. In some embodiments, the z-axis of the biocompatible scaffold is from 900 μm to 1 mm.

In some embodiments, the z-axis of the biocompatible scaffold is 500 μm. In some embodiments, the z-axis of the biocompatible scaffold is 600 μm. In some embodiments, the z-axis of the biocompatible scaffold is 700 μm. In some embodiments, the z-axis of the biocompatible scaffold is 800 μm. In some embodiments, the z-axis of the biocompatible scaffold is 900 μm. In some embodiments, the z-axis of the biocompatible scaffold is 1 mm. In some embodiments, the z-axis of the biocompatible scaffold is 2 mm. In some embodiments, the z-axis of the biocompatible scaffold is 3 mm. In some embodiments, the z-axis of the biocompatible scaffold is 4 mm. In some embodiments, the z-axis of the biocompatible scaffold is 5 mm.

In some embodiments, the biocompatible scaffold polymerizes in less than 3 hours (e.g., less than 2 hours, 1 hour, 59 minutes, 58 minutes, 57 minutes, 56 minutes, 55 minutes, 50 minutes, 45 minutes, 40 minutes, 30 minutes, 20 minutes, 10 minutes, or 5 minutes). For example, in some embodiments, the biocompatible scaffold polymerizes in less than 2 hours. In some embodiments, the biocompatible scaffold polymerizes in less than 1 hour. In some embodiments, the biocompatible scaffold polymerizes in less than 59 minutes. In some embodiments, the biocompatible scaffold polymerizes in less than 58 minutes. In some embodiments, the biocompatible scaffold polymerizes in less than 57 minutes. In some embodiments, the biocompatible scaffold polymerizes in less than 56 minutes. In some embodiments, the biocompatible scaffold polymerizes in less than 55 minutes. In some embodiments, the biocompatible scaffold polymerizes in less than 50 minutes. In some embodiments, the biocompatible scaffold polymerizes in less than 45 minutes. In some embodiments, the biocompatible scaffold polymerizes in less than 40 minutes. In some embodiments, the biocompatible scaffold polymerizes in less than 30 minutes. In some embodiments, the biocompatible scaffold polymerizes in less than 20 minutes. In some embodiments, the biocompatible scaffold polymerizes in less than 10 minutes. In some embodiments, the biocompatible scaffold polymerizes in less than 5 minutes.

In some embodiments, the biocompatible scaffold polymerizes in 2 hours. In some embodiments, the biocompatible scaffold polymerizes in 1 hour. In some embodiments, the biocompatible scaffold polymerizes in 59 minutes. In some embodiments, the biocompatible scaffold polymerizes in 58 minutes. In some embodiments, the biocompatible scaffold polymerizes in 57 minutes. In some embodiments, the biocompatible scaffold polymerizes in 56 minutes. In some embodiments, the biocompatible scaffold polymerizes in 55 minutes. In some embodiments, the biocompatible scaffold polymerizes in 54 minutes. In some embodiments, the biocompatible scaffold polymerizes in 53 minutes. In some embodiments, the biocompatible scaffold polymerizes in 52 minutes. In some embodiments, the biocompatible scaffold polymerizes in 51 minutes. In some embodiments, the biocompatible scaffold polymerizes in 50 minutes. In some embodiments, the biocompatible scaffold polymerizes in 49 minutes. In some embodiments, the biocompatible scaffold polymerizes in 48 minutes. In some embodiments, the biocompatible scaffold polymerizes in 47 minutes. In some embodiments, the biocompatible scaffold polymerizes in 46 minutes. In some embodiments, the biocompatible scaffold polymerizes in 45 minutes. In some embodiments, the biocompatible scaffold polymerizes in 44 minutes. In some embodiments, the biocompatible scaffold polymerizes in 43 minutes. In some embodiments, the biocompatible scaffold polymerizes in 42 minutes. In some embodiments, the biocompatible scaffold polymerizes in 41 minutes. In some embodiments, the biocompatible scaffold polymerizes in 40 minutes. In some embodiments, the biocompatible scaffold polymerizes in 39 minutes. In some embodiments, the biocompatible scaffold polymerizes in 38 minutes. In some embodiments, the biocompatible scaffold polymerizes in 37 minutes. In some embodiments, the biocompatible scaffold polymerizes in 36 minutes. In some embodiments, the biocompatible scaffold polymerizes in 35 minutes. In some embodiments, the biocompatible scaffold polymerizes in 34 minutes. In some embodiments, the biocompatible scaffold polymerizes in 33 minutes. In some embodiments, the biocompatible scaffold polymerizes in 32 minutes. In some embodiments, the biocompatible scaffold polymerizes in 31 minutes. In some embodiments, the biocompatible scaffold polymerizes in 30 minutes. In some embodiments, the biocompatible scaffold polymerizes in 29 minutes. In some embodiments, the biocompatible scaffold polymerizes in 28 minutes. In some embodiments, the biocompatible scaffold polymerizes in 27 minutes. In some embodiments, the biocompatible scaffold polymerizes in 26 minutes. In some embodiments, the biocompatible scaffold polymerizes in 25 minutes. In some embodiments, the biocompatible scaffold polymerizes in 20 minutes. In some embodiments, the biocompatible scaffold polymerizes in 10 minutes. In some embodiments, the biocompatible scaffold polymerizes in 5 minutes.

In some embodiments, the biocompatible scaffold polymerizes in 30-60 minutes.

In some embodiments, the ratio of height of the biocompatible scaffold to height of the layer of hepatocyte and stromal cell aggregates in the biocompatible scaffold is from 20:1 to 1:1 (e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1). For example, in some embodiments the ratio of height of the biocompatible scaffold to height of the layer is from 19:1 to 1:1. In some embodiments, the ratio of height of the biocompatible scaffold to height of the layer is from 18:1 to 1:1. In some embodiments, the ratio of height of the biocompatible scaffold to height of the layer is from 17:1 to 1:1. In some embodiments, the ratio of height of the biocompatible scaffold to height of the layer is from 16:1 to 1:1. In some embodiments, the ratio of height of the biocompatible scaffold to height of the layer is from 15:1 to 1:1. In some embodiments, the ratio of height of the biocompatible scaffold to height of the layer is from 14:1 to 1:1. In some embodiments, the ratio of height of the biocompatible scaffold to height of the layer is from 13:1 to 1:1. In some embodiments, the ratio of height of the biocompatible scaffold to height of the layer is from 12:1 to 1:1. In some embodiments, the ratio of height of the biocompatible scaffold to height of the layer is from 11:1 to 1:1. In some embodiments, the ratio of height of the biocompatible scaffold to height of the layer is from 10:1 to 1:1. In some embodiments, the ratio of height of the biocompatible scaffold to height of the layer is from 9:1 to 1:1. In some embodiments, the ratio of height of the biocompatible scaffold to height of the layer is from 8:1 to 1:1. In some embodiments, the ratio of height of the biocompatible scaffold to height of the layer is from 7:1 to 1:1. In some embodiments, the ratio of height of the biocompatible scaffold to height of the layer is from 6:1 to 1:1. In some embodiments, the ratio of height of the biocompatible scaffold to height of the layer is from 5:1 to 1:1. In some embodiments, the ratio of height of the biocompatible scaffold to height of the layer is from 4:1 to 1:1. In some embodiments, the ratio of height of the biocompatible scaffold to height of the layer is from 3:1 to 1:1.

In some embodiments, the ratio of the height the biocompatible scaffold to height of the layer of hepatocyte and stromal cell aggregates in the biocompatible scaffold is 19:1. In some embodiment, the ratio of the height the biocompatible scaffold to height of the layer of hepatocyte and stromal cell aggregates in the biocompatible scaffold is 18:1. In some embodiment, the ratio of the height the biocompatible scaffold to height of the layer of hepatocyte and stromal cell aggregates in the biocompatible scaffold is 17:1. In some embodiment, the ratio of the height the biocompatible scaffold to height of the layer of hepatocyte and stromal cell aggregates in the biocompatible scaffold is 16:1. In some embodiment, the ratio of the height the biocompatible scaffold to height of the layer of hepatocyte and stromal cell aggregates in the biocompatible scaffold is 15:1. In some embodiment, the ratio of the height the biocompatible scaffold to height of the layer of hepatocyte and stromal cell aggregates in the biocompatible scaffold is 14:1. In some embodiment, the ratio of the height the biocompatible scaffold to height of the layer of hepatocyte and stromal cell aggregates in the biocompatible scaffold is 13:1. In some embodiment, the ratio of the height the biocompatible scaffold to height of the layer of hepatocyte and stromal cell aggregates in the biocompatible scaffold is 12:1. In some embodiment, the ratio of the height the biocompatible scaffold to height of the layer of hepatocyte and stromal cell aggregates in the biocompatible scaffold is 11:1. In some embodiment, the ratio of the height the biocompatible scaffold to height of the layer of hepatocyte and stromal cell aggregates in the biocompatible scaffold is 10:1. In some embodiment, the ratio of the height the biocompatible scaffold to height of the layer of hepatocyte and stromal cell aggregates in the biocompatible scaffold is 9:1. In some embodiment, the ratio of the height the biocompatible scaffold to height of the layer of hepatocyte and stromal cell aggregates in the biocompatible scaffold is 8:1. In some embodiment, the ratio of the height the biocompatible scaffold to height of the layer of hepatocyte and stromal cell aggregates in the biocompatible scaffold is 7:1. In some embodiment, the ratio of the height the biocompatible scaffold to height of the layer of hepatocyte and stromal cell aggregates in the biocompatible scaffold is 6:1. In some embodiment, the ratio of the height the biocompatible scaffold to height of the layer of hepatocyte and stromal cell aggregates in the biocompatible scaffold is 5:1. In some embodiment, the ratio of the height the biocompatible scaffold to height of the layer of hepatocyte and stromal cell aggregates in the biocompatible scaffold is 4:1, In some embodiment, the ratio of the height the biocompatible scaffold to height of the layer of hepatocyte and stromal cell aggregates in the biocompatible scaffold is 3:1. In some embodiment, the ratio of the height the biocompatible scaffold to height of the layer of hepatocyte and stromal cell aggregates in the biocompatible scaffold is 2:1. In some embodiments, the ratio of the height the biocompatible scaffold to height of the layer of hepatocyte and stromal cell aggregates in the biocompatible scaffold is 1:1.

In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel that includes the synthetic polymer described herein can bind various bioactive agents, e.g., growth factors, thereby preventing the bioactive agents from diffusing away and maintaining the bioactive agents at a high concentration locally, so that they can act on cells and promote various cell functions.

### Methods for Making Engineered Tissue Constructs

The methods for making an engineered tissue construct described herein involves two cell types: hepatocytes (e.g., primary human hepatocytes (PHH)) and optionally stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). Frozen master cell banks (MCB) were sourced through external suppliers and were received as cryopreserved cells. All cell types are terminally differentiated cells isolated from primary donors obtained with appropriate donor consent for therapeutic use. For example, hepatocytes (e.g., PHH) are obtained from cadaveric donors via collagenase perfusion, Percoll density gradient purification, and subsequent cryopreservation to create an MCB.

Hepatocytes are stored cryopreserved until initiation of a manufacturing build. Prior to accepting the lot as a released MCB, release testing is conducted on hepatocyte (e.g., PHH) candidate MCBs to establish that their performance characteristics meet acceptance criteria for characterization, release, and stability. Stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts)) are, for example, isolated from a single donor of neonatal foreskin by physical separation of dermal and epidermal layers and sequential digestion with dispase and collagenase. After isolation, stromal cells, e.g., fibroblasts (e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts)) are minimally expanded and cryopreserved to create a frozen MCB. Frozen MCBs are shipped to the manufacturing site and stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts)) are expanded to create working cell banks (WCB), which are then cryopreserved until initiation of a manufacturing build. These WCB are released based on specific acceptance criteria prior to use in the manufacturing process.

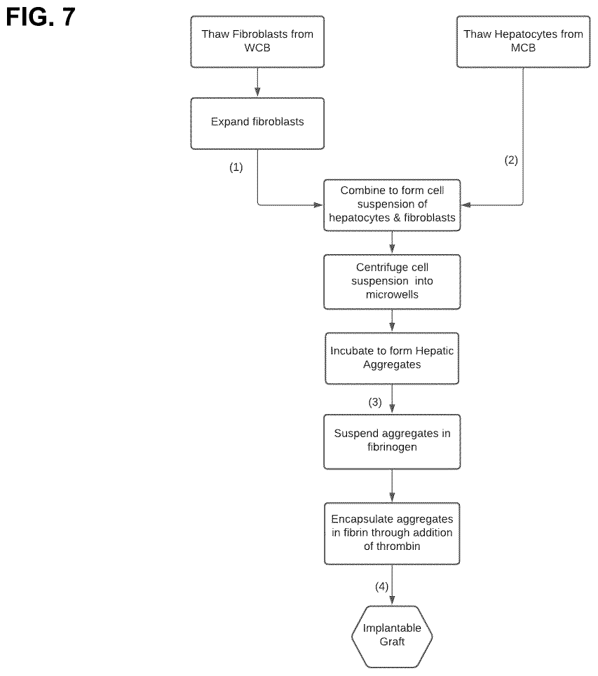

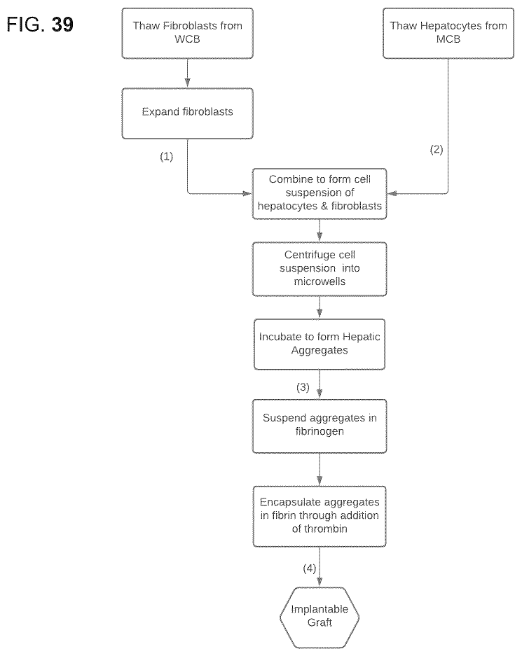

An overview of the continuous manufacturing process for a build is shown in FIG. 7 and FIG. 39. Upon initiation of a manufacturing build, stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts) are thawed from their respective WCB (FIG. 7; Step 1), expanded, and tested to measure viability and cell count. Hepatocytes (e.g., PHH) are thawed from the hepatocyte MCB and tested to measure viability and cell count (FIG. 7; Step 2) prior to optionally being combined at a ratio (e.g., 1:2) with stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts), centrifuged into arrays of microwells (e.g., pyramidal microwells), and incubated for 2-3 days to promote self-assembly of the cells into multicellular hepatic aggregates (e.g., spheroidal aggregates). Hepatic aggregates (e.g., spheroidal aggregates) are deemed acceptable for encapsulation after microscopic confirmation of compaction.

The hepatocyte and optional stromal cell aggregates are then encapsulated with a solution (e.g., a fibrinogen solution) that is polymerized (e.g., with thrombin; FIG. 7; Step 6). These encapsulation steps occur within a mold (e.g., a cylindrical mold) that controls the overall dimensions of the engineered tissue construct to be about 500 μm to 5 mm in thickness, e.g., about 2 mm in thickness, and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension and targeted to be about 2 mm in thickness.

In some embodiments, the mold may be any shape (e.g., cylindrical, square, or square with rounded corners).

Within the solution (e.g., a fibrinogen solution that is polymerized e.g., with thrombin), the hepatocyte/stromal cell aggregates are allowed to non-homogenously distribute (e.g., by gravity) along the z-axis of the biocompatible scaffold into a layer (e.g., to settle), thereby forming a one-sided engineered tissue construct.

In some embodiments, two or more of the one-sided engineered tissue constructs are assembled with each of the layers facing outwardly, respectively, thereby forming a two-sided engineered tissue construct.

The engineered tissue constructs of the present disclosure can be formed by a process described herein. In some embodiments, engineered tissue constructs with defined cellular configurations in a biocompatible hydrogel scaffold may be prepared by photopatterning PEG hydrogels containing the cell populations (e.g., hepatocyte and fibroblast cell populations), resulting in a hydrogel network consisting of 3D cell hepatocytes and stromal cells (e.g., fibroblasts). Further control of cell orientation within these patterned domains may be achieved utilizing dielectrophoretic patterning techniques. Dielectrophoresis (DEP) can be used alone for patterning of cells in relatively homogeneous slabs of hydrogel or in conjunction with the photopolymerization method.

In some embodiments, organizing cells and material into spatial arrangements, such as engineered tissue constructs, can be accomplished by physically constraining the placement of cells/material by the use of wells or grooves, or injecting cells into microfluidic channels or oriented void spaces/pores. In certain embodiments, the cells can be organized by physically positioning cells with electric fields, magnetic tweezers, optical tweezers, ultrasound waves, pressure waves, or micromanipulators. In some embodiments, the population of hepatocytes and the population of stromal cells are aggregated in spheroids and the spheroids are allowed (e.g., by gravity) to non-homogenously distribute along the z-axis of the biocompatible scaffold into a layer.

In certain embodiments, the method for fabricating engineered tissue constructs and embedding the constructs in extracellular matrix includes (1) generating 3D templates that have been defined with channels or trenches, (2) suspending the population of cells in liquid collagen and centrifuging these cells into the channels of the template, (3) removing excess cell/collagen suspension to allow aggregates to form, and (4) removing aggregates from templates via encapsulation in an extracellular matrix scaffold.

In some embodiments, the method for fabricating the engineered tissue constructs includes (1) suspending the population of cells in a naturally-derived and/or synthetic scaffolding, (2) placing the suspended cells into the channels of a 3D template, and (3) allowing the cells to form one or more aggregates at least partially embedded in the naturally-derived and/or synthetic scaffolding. In some embodiments, the 3D template can be generated by molding, templating, photolithography, printing, deposition, sacrificial molding, stereolithography, or a combination thereof.

In some embodiments, an engineered tissue construct can be fabricated through the use a custom 3D printer technology to extrude lattices of carbohydrate glass filaments with predefined diameters, spacings and orientations. For example, in some embodiments, soluble (clinical-grade, sterile) fibrinogen and thrombin are combined and poured over the lattice. After the solution has polymerized into insoluble fibrin, the carbohydrate filaments are dissolved, leaving behind channels within the fibrin. The channels can then be filled with a suspension of cells in a naturally derived or synthetic scaffolding (e.g., soluble type I collagen) that subsequently is polymerized to trap the cells within the channels.

The methods allow for the formation of three-dimensional scaffolds from hundreds of micrometers to tens of centimeters in length and width, and tens of micrometers to hundreds of micrometers in height. A resolution of up to 100 micrometers in the photopolymerization method and possible single cell resolution (10 μm) in the DEP method is achievable. Photopolymerization apparatus, DEP apparatus, and other methods to produce 3-dimensional co-cultures are described in U.S. Pat. No. 8,906,684, which is incorporated herein by reference.

The cells can be cultured in vitro under various culture conditions. The cells can be expanded in culture, e.g., grown under conditions that promote their proliferation. Culture medium can be liquid or semi-solid, e.g., containing agar, methylcellulose, etc. The cell population can be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g., penicillin and streptomycin. The culture can contain growth factors to which the regulatory T cells are responsive. Growth factors, as defined herein, can be molecules capable of promoting survival, growth and/or differentiation of cells, either in culture or in the intact tissue, through specific effects on a transmembrane receptor. Growth factors include polypeptides and non-polypeptide factors.

The cells produced by the methods described herein can be used immediately, e.g., in the making of an engineered tissue construct. Alternatively, the cells can be frozen at liquid nitrogen temperatures and stored for long periods of time, being thawed and capable of being reused. For example, the cells can be frozen in 10% dimethylsulfoxide (DMSO), 50% serum, 40% buffered medium, or some other such solution as is commonly used in the art to preserve cells at such freezing temperatures and thawed in a manner as commonly known in the art for thawing frozen cultured cells.

### Implantation of Engineered Tissue Constructs

The engineered cellular compositions described herein can be implanted in a subject. Non-limiting examples of nonhuman subjects include non-human primates, dogs, cats, mice, rats, guinea pigs, rabbits, fowl, pigs, horses, cows, goats, or sheep. In certain embodiments, the subject can be any animal. In certain embodiments, the subject can be any mammal. In certain embodiments, the subject can be a human.

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat or extraperitoneal fat pad, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the mesentery is the small intestinal mesentery. In some embodiments, the engineered tissue construct is implanted into the subject as a pedicled omental wrap or an omental wrap. In some embodiments, the implantation site is a site that is suitable for neovascularization.

In some embodiments, one or more engineered tissue constructs is implanted at one or more implantation sites selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. For example, in some embodiments, two engineered tissue constructs are implanted bilaterally at the omentum.

The engineered tissue construct can be implanted in any suitable manner, often with pharmaceutically acceptable carriers. In some embodiments, the engineered tissue construct is implanted at the site of a tissue or organ. In some embodiments, the engineered tissue construct is implanted at an orthotopic site. In other embodiments, the engineered tissue construct is implanted at an ectopic site.

In some embodiments, the engineered tissue construct is implanted at any site that is suitable for neovascularization. In some embodiments, a site that it suitable for neovascularization includes a site with a microvessel density of from about 3.6 vessels/mm² to about 4500 vessels/mm² (e.g., 3.7 vessels/mm² to about 4000 vessels/mm², 3.8 vessels/mm² to about 3500 vessels/mm², 3.9 vessels/mm² to about 3000 vessels/mm², 4 vessels/mm² to about 2500 vessels/mm², 5 vessels/mm² to about 2000 vessels/mm², 10 vessels/mm² to about 1000 vessels/mm², or about 100 vessels/mm²).

In some embodiments, a site that is suitable for neovascularization may have an existing microvessel density of greater than about 3.7 vessels/mm². In some embodiments, a site that is suitable for neovascularization may have an existing microvessel density of greater than about 3.8 vessels/mm². In some embodiments, a site that is suitable for neovascularization may have an existing microvessel density of greater than about 3.9 vessels/mm². In some embodiments, a site that is suitable for neovascularization may have an existing microvessel density of greater than about 4 vessels/mm². In some embodiments, a site that is suitable for neovascularization may have an existing microvessel density of greater than about 4.1 vessels/mm². In some embodiments, a site that is suitable for neovascularization may have an existing microvessel density of greater than about 4.2 vessels/mm². In some embodiments, a site that is suitable for neovascularization may have an existing microvessel density of greater than about 4.3 vessels/mm². In some embodiments, a site that is suitable for neovascularization may have an existing microvessel density of greater than about 4.4 vessels/mm². In some embodiments, a site that is suitable for neovascularization may have an existing microvessel density of greater than about 4.5 vessels/mm². In some embodiments, a site that is suitable for neovascularization may have an existing microvessel density of greater than about 5 vessels/mm². In some embodiments, a site that is suitable for neovascularization may have an existing microvessel density of greater than about 6 vessels/mm². In some embodiments, a site that is suitable for neovascularization may have an existing microvessel density of greater than about 7 vessels/mm². In some embodiments, a site that is suitable for neovascularization may have an existing microvessel density of greater than about 8 vessels/mm². In some embodiments, a site that is suitable for neovascularization may have an existing microvessel density of greater than about 9 vessels/mm². In some embodiments, a site that is suitable for neovascularization may have an existing microvessel density of greater than about 10 vessels/mm². In some embodiments, a site that is suitable for neovascularization may have an existing microvessel density of greater than about 50 vessels/mm². In some embodiments, a site that is suitable for neovascularization may have an existing microvessel density of greater than about 100 vessels/mm². In some embodiments, a site that is suitable for neovascularization may have an existing microvessel density of greater than about 200 vessels/mm². In some embodiments, a site that is suitable for neovascularization may have an existing microvessel density of greater than about 300 vessels/mm². In some embodiments, a site that is suitable for neovascularization may have an existing microvessel density of greater than about 400 vessels/mm². In some embodiments, a site that is suitable for neovascularization may have an existing microvessel density of greater than about 500 vessels/mm². In some embodiments, a site that is suitable for neovascularization may have an existing microvessel density of greater than about 600 vessels/mm². In some embodiments, a site that is suitable for neovascularization may have an existing microvessel density of greater than about 700 vessels/mm². In some embodiments, a site that is suitable for neovascularization may have an existing microvessel density of greater than about 800 vessels/mm². In some embodiments, a site that is suitable for neovascularization may have an existing microvessel density of greater than about 900 vessels/mm². In some embodiments, a site that is suitable for neovascularization may have an existing microvessel density of greater than about 1000 vessels/mm². In some embodiments, a site that is suitable for neovascularization may have an existing microvessel density of greater than about 2000 vessels/mm². In some embodiments, a site that is suitable for neovascularization may have an existing microvessel density of greater than about 3000 vessels/mm². In some embodiments, a site that is suitable for neovascularization may have an existing microvessel density of greater than about 4000 vessels/mm². In some embodiments, a site that is suitable for neovascularization may have an existing microvessel density of greater than about 4100 vessels/mm². In some embodiments, a site that is suitable for neovascularization may have an existing microvessel density of greater than about 4200 vessels/mm². In some embodiments, a site that is suitable for neovascularization may have an existing microvessel density of greater than about 4300 vessels/mm². In some embodiments, a site that is suitable for neovascularization may have an existing microvessel density of greater than about 4400 vessels/mm². In some embodiments, a site that is suitable for neovascularization may have an existing microvessel density of greater than about 4500 vessels/mm².

Autologous, allogenic or xenogenic cells may be used. The cells may be implanted in any physiologically acceptable medium. In one embodiment, the cells are cryopreserved in 5-20% DMSO, 5% dextrose and autologous serum. As is familiar to those of skill in the art, dosage of the cells of the present invention to be implanted in vivo is determined with reference to various parameters, including the species of the host, the age, weight, and disease status. Dosage also depends upon the location to be targeted within the subject. For example, implantation of the engineered tissue construct into the omentum may require different dosages than implantation to the mesentery. The dosage is preferably chosen so that implantation causes an effective result, which can be measured by molecular assays (e.g., a liver function test) or by monitoring a suitable symptom in the subject (e.g., symptoms of ALF, Crigler-Najjar, or hyperbilirubinemia). In some embodiments, the method further includes administering an immunosuppressive or immunomodulatory drug to modulate an immune response. In some embodiments, the immune response is a humoral response or antibody-mediated response.

In some embodiments, the implantation method prevents graft rejection or promotes graft survival.

The engineered tissue constructs disclosed herein can be administered in combination with one or more additional immunosuppressive therapies including; but not limited to drugs which inhibit T-cell activation (e.g.; calcineurin inhibitors (CNI)); systemic immunosuppressants for universal transplant immunotolerance (corticosteroids such as methylprednisolone (MEDROL® or; SOLU-MEDROL®); prednisone or prednisolone); CNI such as tacrolimus (PROGRAF® or; ASTAFRAFR); cyclosporine (NEORAL®; SANDIMMUNE® or; GENGRAF®); co-stimulation blockade therapy such as Abatacept (ORENCIA®) and Belatacept (NULOJIX®); anti-metabolites such as Mycophenolate motefil (CELLCEPT® or; MYFORTIC®); Azathioprine (IMURAN®); mTORI such as Sirolimus (RAPAMUNE®); Everolimus (AFINITOR®); T-cell depleting monoclonal antibodies such as muromonab-CD3 (OKT3); Alemtuzumab (Campath® or; LEMTRADA®); ATG (THYMOBLOBULIN® or; ATGAM®); B-cell depleting monoclonal antibodies such as rituximab (RITUXAN®); proteasome inhibitors such as Bortezomib (VELCADE®); IL-2-Ra monoclonal antibodies such as daclizumab (ZENAPAX®); Basiliximab (SIMULECT®); lymphocyte integrin blockade monoclonal antibodies such as Natalizumab (TYSABRI®); N-Acetyl Cysteine (NAC); hepatitis B vaccine (HEPLISAV-B®); glecaprevir and pibrentasvir (MAVYRET®); sofosbuvir (VOSEVI®); obeticholic acid (OCALIVA®); elbasvir and grazoprevir (ZEPATIER®); cholic acid (CHOLBAM®); daclatasvir (DAKLINZA®); ombitasvir, paritaprevir and ritonavir (TECHNIVIE™); simeprevir (OLYSIO™); sofosbuvir (SOVALDI®); telaprevir (INCIVEK™); boceprevir (VICTRELIS™); tenofovir disoproxil fumarate (VIREAD®); telbivudine (TYZEKA™); entecavir (BARACLUDE™); adefovir (HEPSERA®); peginterferon alfa-2a (PEGASYS®); peginterferon alfa-2b (PEGINTRON®); or ribavirin and twinrix. Additional agents include gliltazones and vitamin E.

In some embodiments, the engineered tissue constructs disclosed herein can be administered in combination with one or more additional immunosuppressive therapies including but not limited to a PEGylated anti-CD28 monovalent monoclonal antibody fragment (e.g., anti-human CD28 FR10⁴) or domain antibody such as lulizumab (BMS-931699), an IL-2Ra specific antibody for Treg expansion (e.g., a Fc IL-2 mutein (e.g., AMG-592)), a PEGylated IL-2 antibody, a humanized IgG1 anti-CD40L antagonist (e.g., AT-1501), a bivalent anti-CD40L domain antibody such as letolizumab (BMS-986004), an Fc silent human IgG1 anti-CD40 antibody such as VIB4920 or iscalimab (CFZ533), imlifidase, or a human anti-IL6 monoclonal antibody such as clazakizumab (CSL300).

In some embodiments, following implantation of the engineered tissue construct, the engineered tissue construct persists for at least two weeks (e.g., at least three weeks, one month, two months, three months, four months, five months, six months, seven months, ten months, eleven months, one year, five years, ten years, or the lifetime of a patient in which the engineered tissue construct is implanted into). For example, in some embodiments, following implantation of the engineered tissue construct, the engineered tissue construct persists for at least three weeks. In some embodiments, following implantation of the engineered tissue construct, the engineered tissue construct persists for at least one month. In some embodiments, following implantation of the engineered tissue construct, the engineered tissue construct persists for at least two months. In some embodiments, following implantation of the engineered tissue construct, the engineered tissue construct persists for at least three months. In some embodiments, following implantation of the engineered tissue construct, the engineered tissue construct persists for at least four months. In some embodiments, following implantation of the engineered tissue construct, the engineered tissue construct persists for at least five months. In some embodiments, following implantation of the engineered tissue construct, the engineered tissue construct persists for at least six months. In some embodiments, following implantation of the engineered tissue construct, the engineered tissue construct persists for at least seven months. In some embodiments, following implantation of the engineered tissue construct, the engineered tissue construct persists for at least eight months. In some embodiments, following implantation of the engineered tissue construct, the engineered tissue construct persists for at least nine months. In some embodiments, following implantation of the engineered tissue construct, the engineered tissue construct persists for at least ten months. In some embodiments, following implantation of the engineered tissue construct, the engineered tissue construct persists for at least eleven months. In some embodiments, following implantation of the engineered tissue construct, the engineered tissue construct persists for at least one year. In some embodiments, following implantation of the engineered tissue construct, the engineered tissue construct persists for at least five years. In some embodiments, following implantation of the engineered tissue construct, the engineered tissue construct persists for at least ten years. In some embodiments, following implantation of the engineered tissue construct, the engineered tissue construct persists for the lifetime of a patient in which the engineered tissue construct is implanted into.

### Recommended Clinical Parameters for Monitoring Following Implantation of the Engineered Tissue Construct

Following implantation of the engineered tissue construct, the subject may exhibit a change in one or more clinical parameters. For example, in some embodiments, following implantation of the engineered tissue construct, the subject exhibits a level of serum ammonia in the age-adjusted norm of less than or equal to about 80 μmol/L (e.g., less than about 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L). Alternatively, for example, in some embodiments, following implantation of the engineered tissue construct, the subject exhibits a level of serum ammonia of less than or equal to about 500 μmol/L (e.g., less than about 499 μmol/L, 488 μmol/L, 487 μmol/L, 486 μmol/L, 485 μmol/L, 480 μmol/L, 470 μmol/L, 460 μmol/L, 450 μmol/L, 400 μmol/L, 300 μmol/L, 200 μmol/L, 100 μmol/L, 50 μmol/L, 40 μmol/L, 30 μmol/L, 20 μmol/L, or 10 μmol/L).

In some embodiments, following implantation of the engineered tissue construct, the subject exhibits an improvement in a test of gallbladder ejection fraction (e.g., a hepatobiliary iminodiacetic acid scan). As yet another example, in some embodiments, following implantation of the engineered tissue construct, the subject exhibits a change in one or more parameters in a blood test relative to a reference level. For example, in some embodiments, following implantation of the engineered tissue construct, the subject exhibits a change in one or more parameters in a blood test relative to a reference level.

### Blood Test

Following implantation of the engineered tissue construct, the subject may exhibit a change in one or more parameters in a blood test (e.g., a liver function test (LFT), an ammonia test, or a bilirubin test). In some embodiments, the subject exhibits a change in one or more parameters (e.g., albumin, gamma-glutamyl transferase (GGT) level, alkaline phosphatase (ASP) level, aspartate aminotransferase (AST) level, alanine aminotransferase (ALT level), or bilirubin level) in a blood test relative to a reference level following implantation of the engineered tissue construct.

Following implantation of the engineered tissue construct described herein, the subject may exhibit a change in one or more parameters in a blood test (e.g., a bilirubin test). In some embodiments, the subject exhibits a change in one or more parameters (e.g., bilirubin level) in a blood test relative to a reference level following implantation of the engineered tissue construct.

### Albumin

In some embodiments, following implantation of the engineered tissue construct, the subject exhibits a change in the level of albumin, which can be measured with a LFT. For example, in some embodiments, following implantation of the engineered tissue construct, the subject exhibits a change in the level of albumin, such that their albumin level is returned to the age-adjusted norm.

For example, if the subject was a human toddler (e.g., 6-12 months old), it would be determined that a subject exhibits a albumin level that is returned to the age-adjusted norm when the subject's albumin level is within the normal range of about 30-55 U/L (e.g., 31-55 U/L, 32-55 U/L, 33-55 U/L, 34-55 U/L, 35-55 U/L, 36-55 U/L, 37-55 U/L, 38-55 U/L, 39-55 U/L, 40-55 U/L, 41-55 U/L, 42-55 U/L, 43-55 U/L, 44-55 U/L, 45-55 U/L, 46-55 U/L, 47-55 U/L, 48-55 U/L, 49-55 U/L, 50-55 U/L, 51-55 U/L, 52-55 U/L, 53-55 U/L, or 54-55 U/L). Alternatively, for example, if the subject was a human aged 1-45 years old, it would be determined that a subject exhibits a albumin level that is returned to the age-adjusted norm when the subject's albumin level is within the normal range of about 40-50 U/L (e.g., about 41-50 U/L, 42-50 U/L, 43-50 U/L, 44-50 U/L, 45-50 U/L, 46-50 U/L, 47-50 U/L, 48-50 U/L, or 49-50 U/L).

If the subject was a human aged 46-90 years old, it would be determined that a subject exhibits an albumin level that is returned to the age-adjusted norm when the subject's albumin level is within the normal range of about 35-50 U/L (e.g., about 36-50 U/L, 37-50 U/L, 38-50 U/L, 39-50 U/L, 40-50 U/L, 41-50 U/L, 42-50 U/L, 43-50 U/L, 44-50 U/L, 45-50 U/L, 46-50 U/L, 47-50 U/L, 48-50 U/L, or 49-50 U/L).

### Gamma-Glutamyl Transferase

In some embodiments, following implantation of the engineered tissue construct, the subject exhibits a change in the level of GGT, which can be measured with a LFT. For example, in some embodiments, following implantation of the engineered tissue construct, the subject exhibits a change in the level of GGT, such that their GGT level is returned to the age-adjusted norm.

For example, if the subject was a human toddler (e.g., 6-12 months old), it would be determined that a subject exhibits a GGT level that is returned to the age-adjusted norm when the subject's GGT level is within the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

Alternatively, for example, if the subject was a human child aged 1-5 years old, it would be determined that a subject exhibits a GGT level that is returned to the age-adjusted norm when the subject's GGT level is within the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

### Alkaline Phosphatase

In some embodiments, following implantation of the engineered tissue construct, the subject exhibits a change in the level of ASP, which can be measured with a LFT. For example, in some embodiments, following implantation of the engineered tissue construct, the subject exhibits a change in the level of ASP, such that their ASP level is returned to the age-adjusted norm.

For example, if the subject was a human, it would be determined that a subject exhibits an ASP level that is returned to the age-adjusted norm when the subject's ASP level is within the normal range of about 50 to 300 U/L e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

### Aspartate Aminotransferase

In some embodiments, following implantation of the engineered tissue construct, the subject exhibits a change in the level of AST, which can be measured with a LFT. For example, in some embodiments, following implantation of the engineered tissue construct, the subject exhibits a change in the level of AST, such that their AST level is returned to the age-adjusted norm.

For example, if the subject was a human, it would be determined that a subject exhibits a AST level that is returned to the age-adjusted norm when the subject's AST level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, following implantation of the engineered tissue construct, the subject exhibits a change in the level of ALT, which can be measured with a LFT. For example, in some embodiments, following implantation of the engineered tissue construct, the subject exhibits a change in the level of ALT, such that their ALT level is returned to the age-adjusted norm.

For example, if the subject was a human, it would be determined that a subject exhibits a ALT level that is returned to the age-adjusted norm when the subject's ALT level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Bilirubin

In some embodiments, following implantation of the engineered tissue construct, the subject exhibits a change in the level of total bilirubin, which can be measured with a bilirubin test. For example, in some embodiments, following implantation of the engineered tissue construct, the subject exhibits a change in the level of total bilirubin, such that their total bilirubin level is returned to the age-adjusted norm of less than about 1.2 mg/dl (e.g., less than about 1.2 mg/dl, 1.1 mg/dL, 1 mg/dl, 0.9 mg/dl, 0.8 mg/dl, 0.7 mg/dL, 0.6 mg/dl, 0.5 mg/dl, 0.4 mg/dL, 0.3 mg/dl, 0.2 mg/dl, or 0.1 mg/dL).

In some embodiments, following implantation of the engineered tissue construct, the subject exhibits a change in the level of direct bilirubin, which can be measured with a bilirubin test. For example, in some embodiments, following implantation of the engineered tissue construct, the subject exhibits a change in the level of direct bilirubin, such that their direct bilirubin level is returned to the age-adjusted norm of less than about 1.7 mg/dl (e.g., less than about 1.6 mg/dl, 1.5 mg/dl, 1.4 mg/dl, 1.3 mg/dl, 1.2 mg/dL, 1.1 mg/dL, 1 mg/dl, 0.9 mg/dL, 0.8 mg/dl, 0.7 mg/dL, 0.6 mg/dl, 0.5 mg/dl, 0.4 mg/dl, 0.3 mg/dl, 0.2 mg/dL, or 0.1 mg/dL).

In some embodiments, following implantation of the engineered tissue construct, the subject exhibits a change in the level of bilirubin, which can be measured with a bilirubin test. For example, in some embodiments, following implantation of the engineered tissue construct, the subject exhibits a change in the level of bilirubin, such that their bilirubin level is returned to the age-adjusted norm of less than about 1 mg/dl (less than about 0.9 mg/dl, 0.8 mg/dl, 0.7 mg/dL, 0.6 mg/dl, 0.5 mg/dL, 0.4 mg/dl, 0.3 mg/dl, 0.2 mg/dL, or 0.1 mg/dL).

### Kits

The compositions described herein can be provided in a kit. The compositions described herein can be provided in a kit for use in treating ALF or hyperbilirubinemia (e.g., in a subject having Crigler-Najjar syndrome). The kit may include one or more engineered tissue constructs as described herein. The kit can include a package insert that instructs a user of the kit, such as a physician, to perform any one of the methods described herein. The kit can include a package insert that instructs a user of the kit, such as a physician, to implant the engineered tissue construct. The kit may optionally include surgical equipment or another device for administering the composition or for implanting the engineered tissue construct. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. In Vivo Evaluation of Implanted Engineered Tissue Constructs

To determine the effectiveness of engineered tissue constructs in treating acute liver failure (ALF) in an in vivo model, a study was performed with female transgenic thymidine kinase-NOD/Shi-scid/IL-2Ryrul (TK-NOG) (Taconic model #12907-F) mice (4-8 weeks of age upon arrival). TK-NOG mice are immunodeficient mice with transgenic expression of thymidine kinase (TK) under control of a liver-restricted albumin promoter, which provide inducible ablation of hepatocytes by ganciclovir (GCV) treatment. For study design, see Table 1, below.

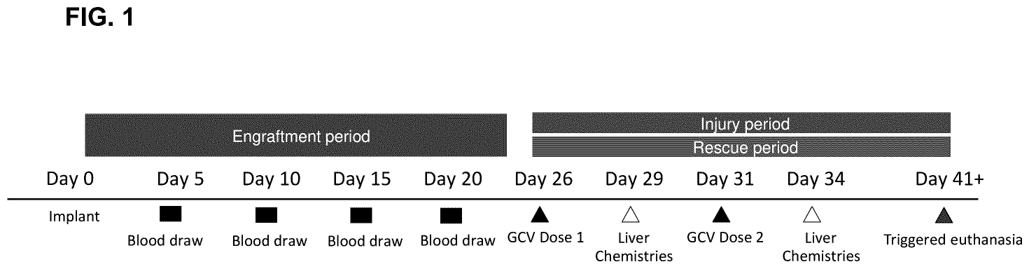

On day 0 of the study, Groups 3 and 5 TK-NOG mice were implanted with two engineered tissue constructs, each consisting of 1.41×10⁶ primary human hepatocytes (PHH) and 2.82×10⁶ normal human dermal fibroblasts for a total of 2.82M PHH/animal. Groups 1, 2, and 4 were not subjected to surgery. Clinical observation, including measurement of body weight, was observed daily. Blood was drawn on days 5, 10, 15, 20, 29, and 34 (with chemistry to determine the levels of liver enzymes performed on the blood drawn from days 29 and 34); GCV or phosphate buffered saline (PBS) was administered intraperitoneally (i.p.) on days 26 and 29 in a dose of 50 mg/kg or 75 mg/kg, respectively; followed by triggered euthanasia (FIGS. 1, 2A, and 3A). Following euthanasia, engineered tissue constructs were explanted and fixed for histological analysis of hepatocyte expression and integration with endothelial cells.

TABLE 1
In vivo evaluation of implanted engineered
tissue constructs study design

| | | | | | Ter- |
|---|---|---|---|---|---|
| | | | Hepatocytes | GCV Dose | minal |
| Group | Number | | Ablation | Level | Time |
| Number | of mice | Treatment | Induction | (mg/kg) | point |
| 1 | 4 | No surgery or | PBS | No treatment | Day |
| | | implantation | | with toxin dose | 42 |
| 2 | 4 | No surgery or | GCV | 50 | |
| | | implantation | | | |
| 3 | 8 | Engineered | | | |
| | | tissue | | | |
| | | construct | | | |
| | | implanted | | | |
| | | Day 1 | | | |
| 4 | 4 | No surgery or | | 75 | |
| | | implantation | | | |
| 5 | 8 | Engineered | | | |
| | | tissue | | | |
| | | construct | | | |
| | | implanted | | | |
| | | Day 1 | | | |

### Results

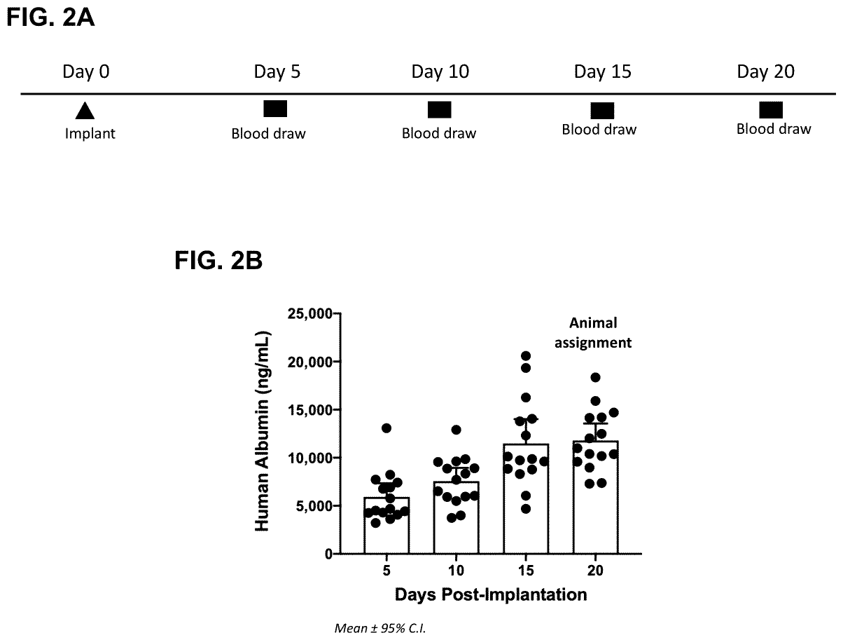

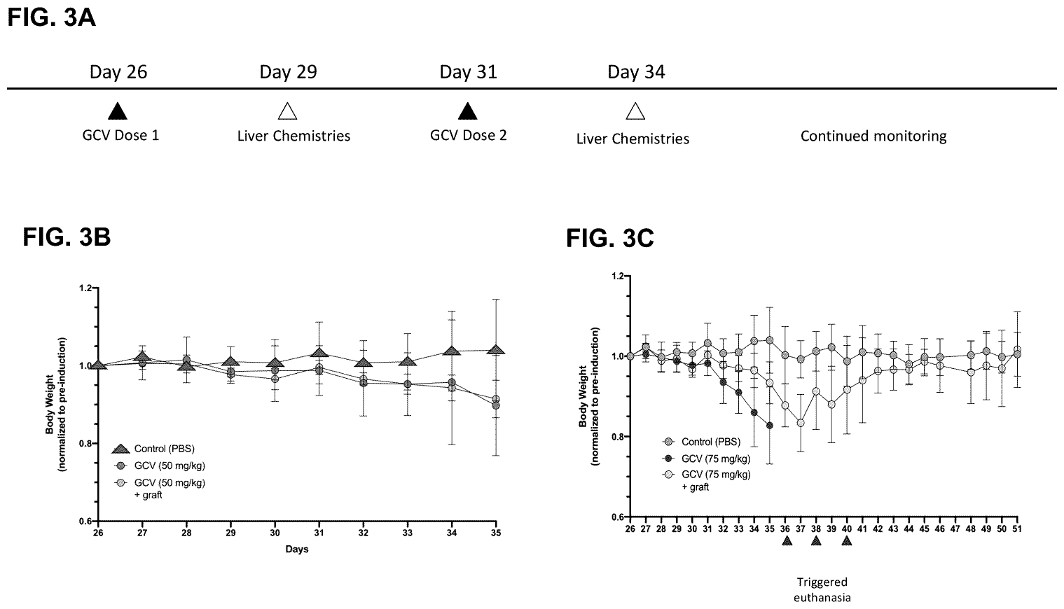

Over time, mice implanted with engineered tissue constructs showed elevated levels of human albumin (ng/ml) (FIG. 2B). In evaluation of the weight of mice over time, it was observed that mice that were treated with 75 mg/kg of GCV and implanted with two engineered tissue constructs (Group 5), exhibited a decrease in weight at about day 34 that was returned to a normalized weight (pre-induction) that was comparable to the no-surgery/implantation and no-toxin controls (Group 1) by about day 42. In contrast, mice that received 75 mg/kg of GCV alone, without surgery or the implantation of an engineered tissue construct (Group 4), required triggered euthanasia, as the decrease in weight associated with hepatocyte ablation by GCV was not attenuated (FIG. 3C). The effect of hepatocyte ablation on the reduction of weight was less apparent in the experimental groups receiving 50 mg/kg of GCV (Groups 2 and 3; FIG. 3B).

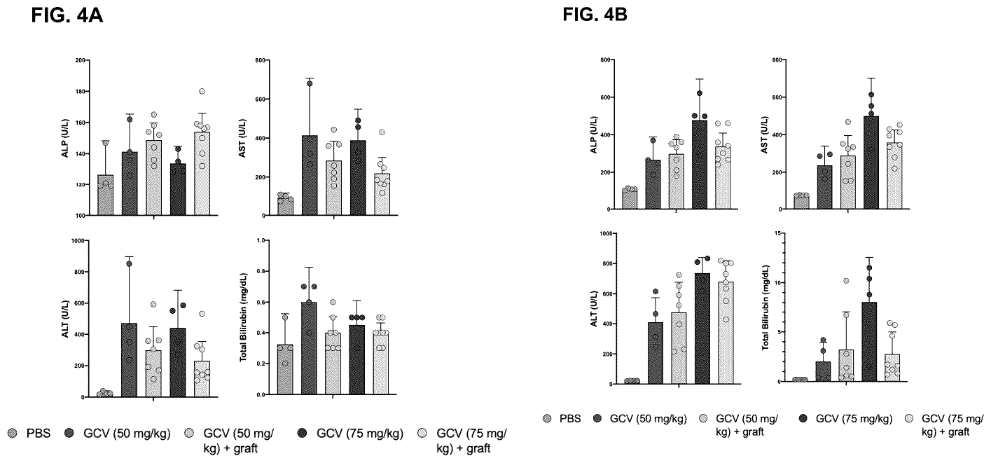

In evaluation of the levels of liver enzymes including alkaline phosphatase (ALP), aspartate aminotransferase (AST), and alanine aminotransferase (ALT); and bilirubin, we observed that implantation of the engineered tissue construct effectively normalized the levels of AST, ALT, and bilirubin by day 29 in mice receiving 50 mg/kg or 75 mg/kg of GCV (FIG. 4A), as compared to the no-surgery and no-toxin control (Group 1), while the increase in ALP associated with hepatocyte ablation was attenuated by implantation of the engineered tissue construct by day 34 in mice receiving 50 mg/kg or 75 mg/kg of GCV (FIG. 4B) as compared to the no-surgery and no-toxin control (Group 1).

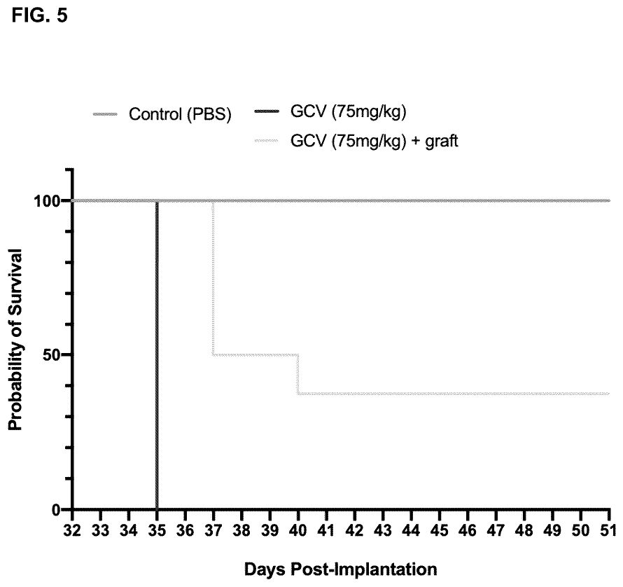

In accordance with the results described above for the weight loss study, in a probability of survival study, we observed that mice that were treated with 75 mg/kg of GCV and implanted with two engineered tissue constructs (Group 5), exhibited an elevated probability to survive, whereas mice that received 75 mg/kg of GCV alone, without surgery or the implantation of an engineered tissue construct (Group 4), had a reduced probability to survive, with a sharp increase in mortality beginning on day 37 (FIG. 5).

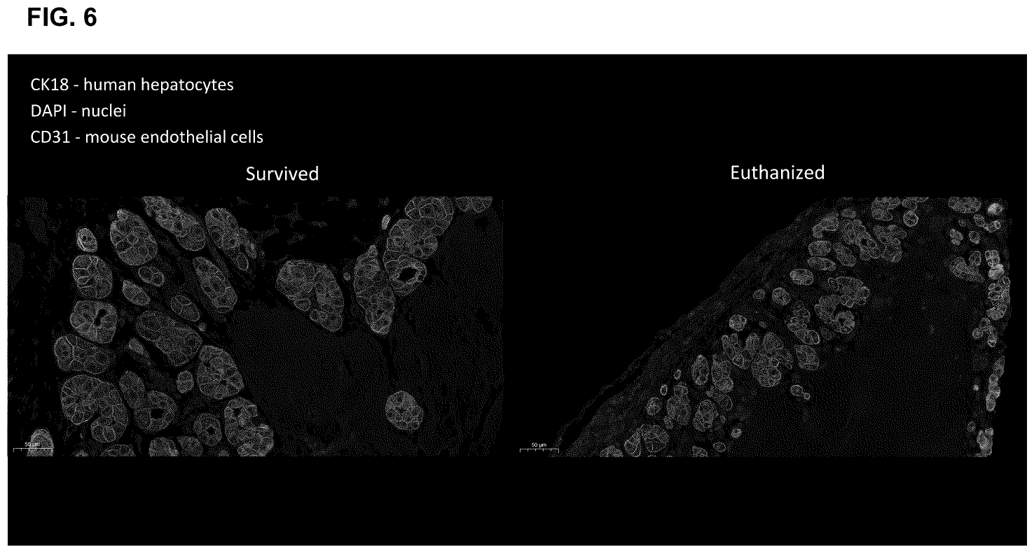

Following triggered euthanasia, histological analyses of the explanted engineered tissue constructs revealed functioning blood vessels (CD31-positive endothelial cells) and a thorough integration of said endothelial cells with human hepatocytes (CK18-positive hepatocytes) (FIG. 6). Taken together, these histological analyses revealed a surprising number of hepatocytes that survived (e.g., persisted) for greater than 31 days and continued to persist over time (e.g., at least three months).

### Example 2. In Vivo Evaluation of Doses of Engineered Tissue Constructs in an Immunodeficient Mouse Model

To determine the effectiveness of different doses of engineered tissue constructs and their ability to mediate prophylactic effects in an in vivo model of acute liver failure, a study was performed with TK-NOG (Taconic model #12907-F) mice (4-8 weeks of age upon arrival).

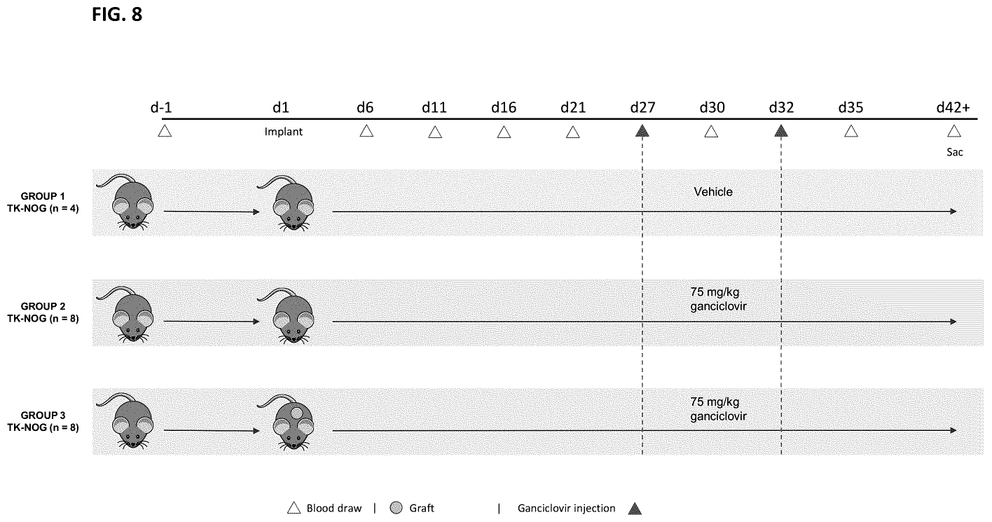

On day −1, each TK-NOG mouse underwent a blood draw. On day 1 mice were implanted with one engineered tissue constructs, resulting in 0.7×10⁶ PHH/mouse (low dose “group 3”) or 5 engineered tissue constructs, resulting in 7×10⁶ PHH/mouse (high dose “group 3”). Following implantation, all mice underwent a blood draw on days 6, 11, 16, 21, 30, 35, and 42; as well as a dosing of GCV on days 27 and 32 (control group 2 and experimental group 3). Mice were sacrificed on day 42. Clinical observations were made daily, and blood was drawn with chemistry to determine the levels of liver enzymes performed on the blood drawn. Furthermore, GCV or phosphate buffered saline (PBS) was administered intraperitoneally (i.p.) (FIG. 8).

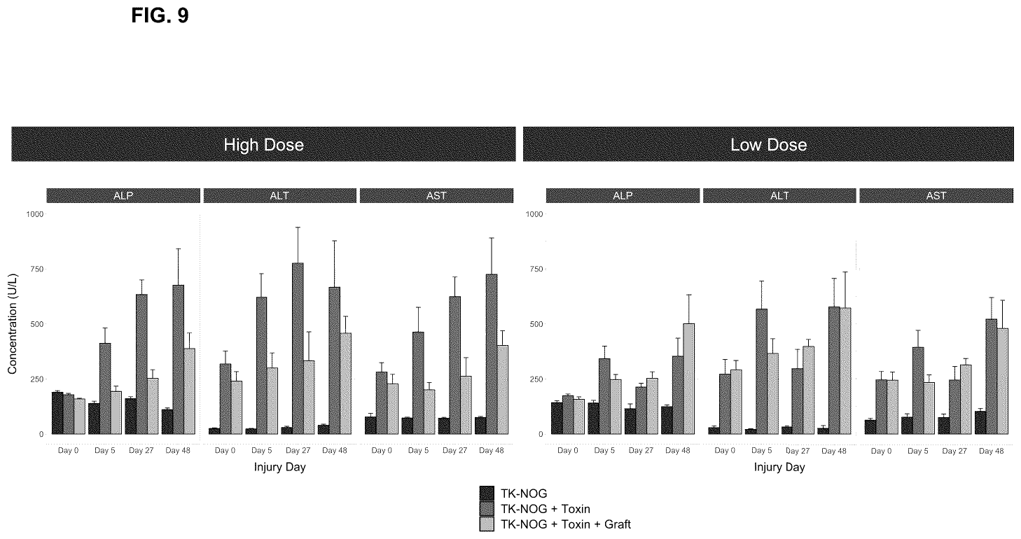

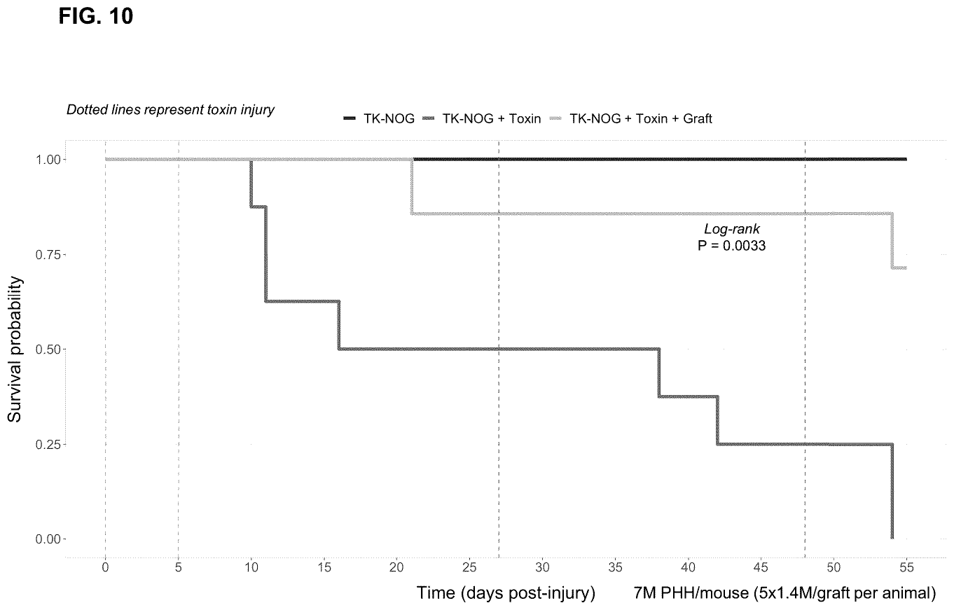

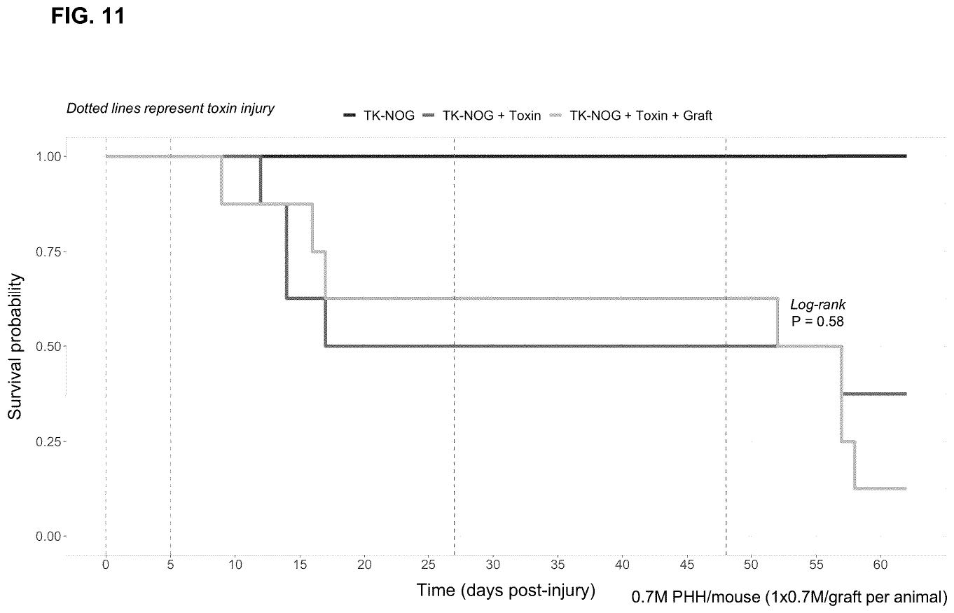

In evaluation of the levels of liver enzymes including alkaline phosphatase (ALP), ALT, and AST, we observed that implantation of the high dose engineered tissue construct effectively normalized the levels of ALP, ALT, and AST, while the low dose engineered tissue construct elicited more modest effects (FIG. 9). In a probability of survival study, we observed that mice that were treated with GCV and implanted with a high-dose engineered tissue construct, exhibited an elevated probability to survive, whereas mice that received GCV alone, without the implantation of an engineered tissue construct, had a reduced probability to survive, with a sharp increase in mortality beginning on day 10 (FIG. 10). The low-dose implant was less effective in attenuating mortality (FIG. 11).

### Example 3. Treatment of Acute Liver Failure in a Human Patient by Implanting an Engineered Tissue construct including hepatocytes and stromal cells

A pediatric patient (age of 1 year old) having acute liver failure (ALF) is treated using an engineered tissue construct. The patient is administered an engineered tissue construct that includes an amount of hepatocytes (e.g., primary human hepatocytes) that is equivalent to 0.5% to 30% of the mass of the liver preserve of the subject and stromal cells (e.g., fibroblasts e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. The patient shows significant improvement in liver function based on improved blood levels of one or more of gamma-glutamyl transferase, alkaline phosphatase, aspartate aminotransferase, alanine aminotransferase, or albumin.

### Example 4. Treatment of Acute Liver Failure in a Human Patient by Implanting an Engineered Tissue Construct Including Hepatocytes and Stromal Cells

A neonate patient (age of 1 day old) having ALF is treated using an engineered tissue construct. The patient is administered an engineered tissue construct that includes from about 3×10⁵ to about 3×10¹⁰ (e.g., 1×10⁶ to about 1×10¹⁰, or 1×10⁷ to about 1×10⁹, or about 1×10⁸) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 5. Treatment of Acute Liver Failure in a Human Patient by Implanting an Engineered Tissue Construct Including Hepatocytes and Stromal Cells

A patient (age of 18 years old) having ALF is treated using an engineered tissue construct. The patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 6. Treatment of Acute Liver Failure in a Human Patient by Implanting an Engineered Tissue Construct Including Hepatocytes and Stromal Cells

A pediatric patient (age of 9 years old) having ALF is treated using an engineered tissue construct. The patient is administered an engineered tissue construct that includes from about 4.5×10⁷ to about 1.35×10¹¹ (e.g., 5×10⁷ to about 1×10¹¹, 1×10⁸ to about 1×10¹⁰, or about 1×10⁹) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 7. Treatment of Acute Liver Failure in a Human Patient by Implanting an Engineered Tissue Construct Including Hepatocytes and Stromal Cells

A patient (age of 60 years old) having ALF is treated using an engineered tissue construct. The patient is administered an engineered tissue construct that includes an amount of hepatocytes e.g., primary human hepatocytes) that is equivalent to 0.5% to 30% of the total liver mass of the subject and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 8. Treatment of Acute Liver Failure in a Human Patient by Implanting an Engineered Tissue Construct Including Hepatocytes and Stromal Cells

A patient (age of 30 years old) having ALF is treated using an engineered tissue construct. The patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 9. Engineered Tissue Construct Manufacturing

Engineered tissue constructs were manufactured with primary adult human hepatocytes (PHH) and neonatal human dermal fibroblasts (NHDF). Frozen master cell banks were sourced through external suppliers and were received as cryopreserved cells. All cell types are terminally differentiated cells isolated from primary donors obtained with appropriate donor consent for therapeutic use. PHH are obtained from cadaveric donors via collagenase perfusion, Percoll density gradient purification, and subsequent cryopreservation to create a master cell bank (MCB). Hepatocytes are stored cryopreserved until initiation of a manufacturing build. Prior to accepting the lot as a released MCB, release testing is conducted on PHH candidate MCBs to establish that their performance characteristics meet acceptance criteria for characterization, release and stability. NHDF are isolated from a single donor of neonatal foreskin by physical separation of dermal and epidermal layers and sequential digestion with dispase and collagenase. After isolation, NHDF are minimally expanded and cryopreserved to create a frozen MCB. Frozen MCBs are shipped to Satellite Bio and NHDF are expanded to create working cell banks (WCB) and then cryopreserved until initiation of a manufacturing build. These WCB are released based on specific acceptance criteria prior to use in the manufacturing process.

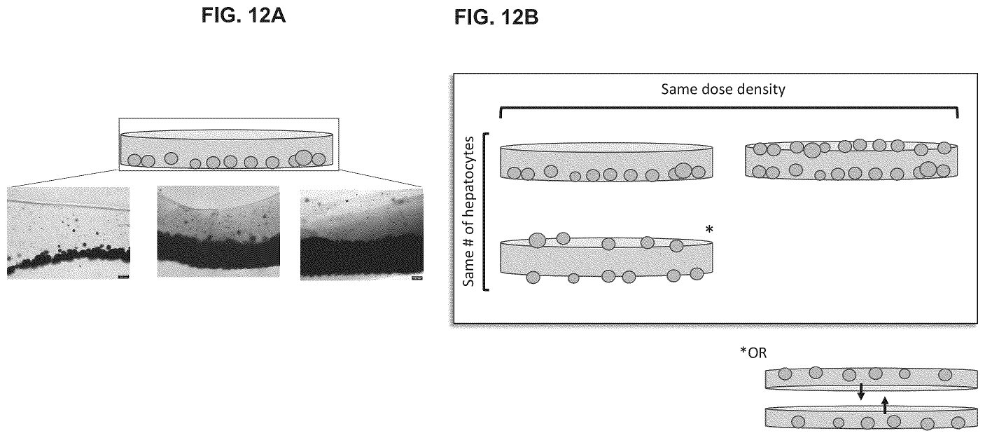

Upon initiation of a manufacturing build, NHDF are thawed from their respective WCB, expanded and tested to measure viability and cell count. PHH are thawed from the PHH MCB and tested to measure viability and cell count prior to being combined at a 1:2 ratio with NHDF, centrifuged into arrays of pyramidal microwells and incubated for 2-3 days to promote self-assembly of the cells into multicellular hepatic aggregates. Hepatic aggregates are deemed acceptable for encapsulation after microscopic confirmation of compaction (FIG. 12A).

The hepatocyte/stromal cell aggregates are then encapsulated with a fibrinogen solution that is polymerized with thrombin. These encapsulation steps occur within a cylindrical mold that controls the overall dimensions of the graft to be 2 mm in thickness and an outer diameter of 6 mm-200 mm. The thickness is controlled by the volume of cell-hydrogel suspension and targets 2 mm in thickness.

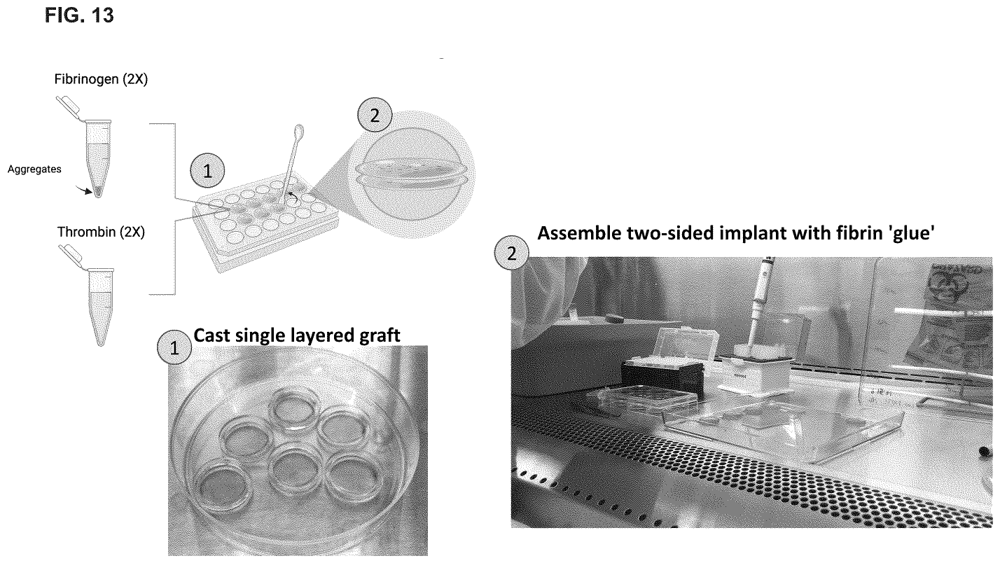

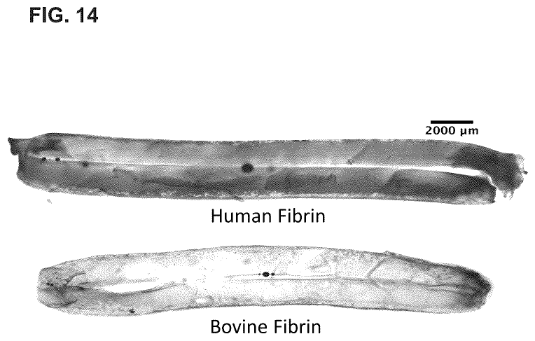

Furthermore, simultaneous with the polymerization step, the aggregates are allowed to distribute nonhomogeneously (e.g., via gravity into a layer at the bottom). Afterwards, one or more engineered tissue constructs may be assembled with biocompatible glue (e.g., fibrin glue) such that each of the aggregate layers is outward facing, thereby forming a two-sided engineered tissue construct (FIG. 12B, FIG. 13, and FIG. 14).

### Example 10. In Vivo Evaluation of Engineered Tissue Constructs in Two Immunodeficient Mouse models

To determine the effectiveness of engineered tissue constructs in an in vivo model of acute liver failure, a study was performed with NOD-scid IL2Rgamma^{null} (NSG™) mice (4-8 weeks of age upon arrival). NSG™ mice are immunodeficient mice.

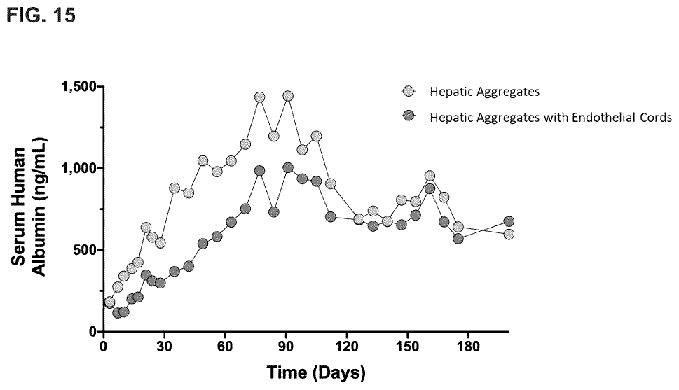

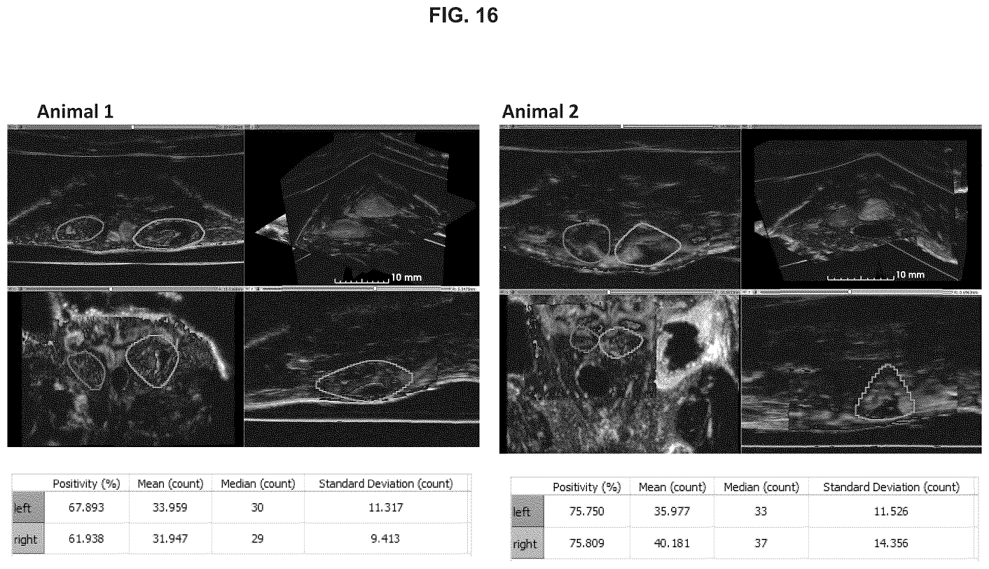

On day 0 of the study, NSG™ mice were implanted with two engineered tissue constructs, each consisting of 1.41×10⁶ PHH and 2.82×10⁶ NHDFS for a total of 2.82M PHH/animal, with or without endothelial cells. Over time, mice implanted with engineered tissue constructs without endothelial cells (“Hepatic Aggregates”) showed elevated levels of human albumin (ng/ml), as compared to the experimental group with endothelial cells (“Hepatic Aggregates with Endothelial Cords”) (FIG. 15). The beneficial effect of engineered tissue constructs lacking endothelial cells was also apparent in the vascular volume of the implantation site, using SonoVol imaging (FIG. 16).

A second study was performed with transgenic thymidine kinase-NOD/Shi-scid/IL-2Rynul (TK-NOG) (Taconic model #12907-F) mice (4-8 weeks of age upon arrival). TK-NOG mice are immunodeficient mice with transgenic expression of thymidine kinase (TK) under control of a liver-restricted albumin promoter, which provide inducible ablation of hepatocytes by ganciclovir (GCV) treatment.

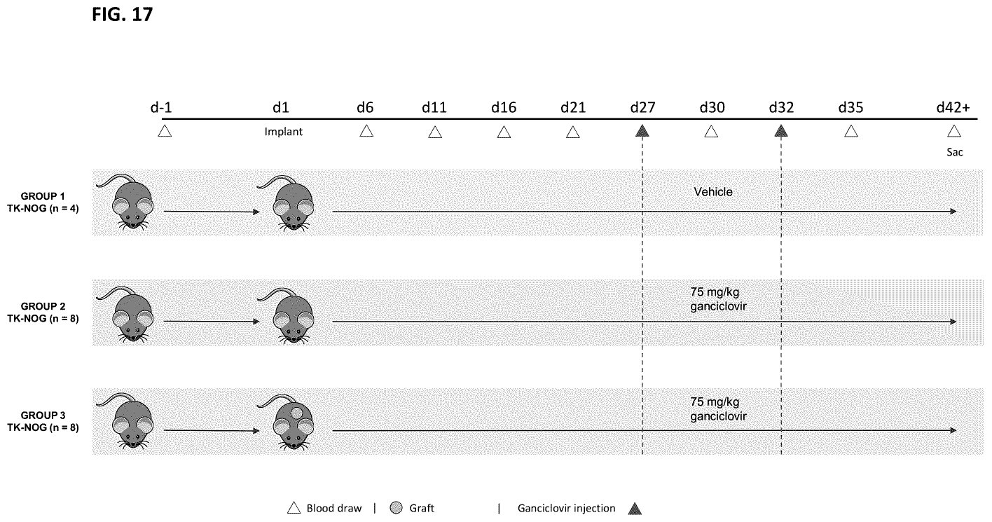

On day −1, each TK-NOG mouse underwent a blood draw. On day 1 mice were implanted with one engineered tissue constructs, resulting in 0.7×10⁶ PHH/mouse (low dose “group 3”) or 5 engineered tissue constructs, resulting in 7×10⁶ PHH/mouse (high dose “group 3”). Following implantation, all mice underwent a blood draw on days 6, 11, 16, 21, 30, 35, and 42; as well as a dosing of GCV on days 27 and 32 (control group 2 and experimental group 3). Mice were sacrificed on day 42. Clinical observations were made daily, and blood was drawn with chemistry to determine the levels of liver enzymes performed on the blood drawn. Furthermore, GCV or phosphate buffered saline (PBS) was administered intraperitoneally (i.p.) (FIG. 17).

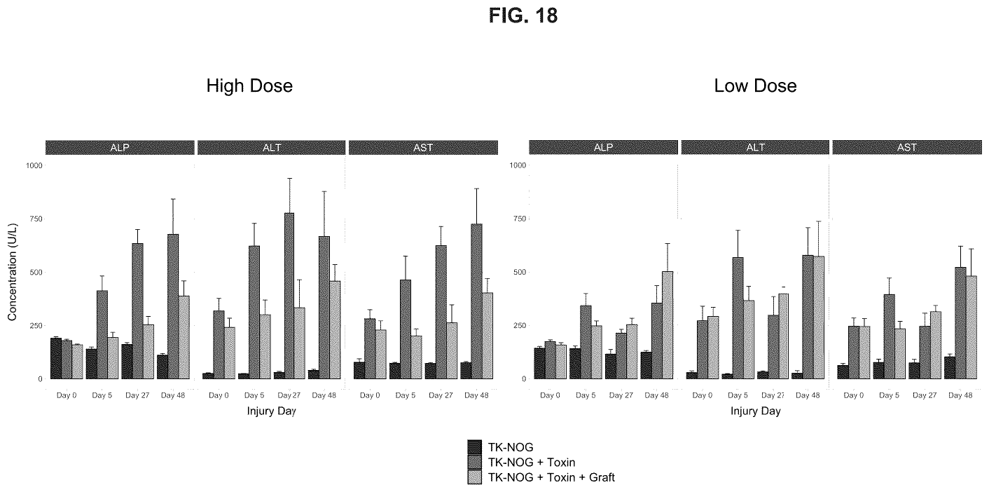

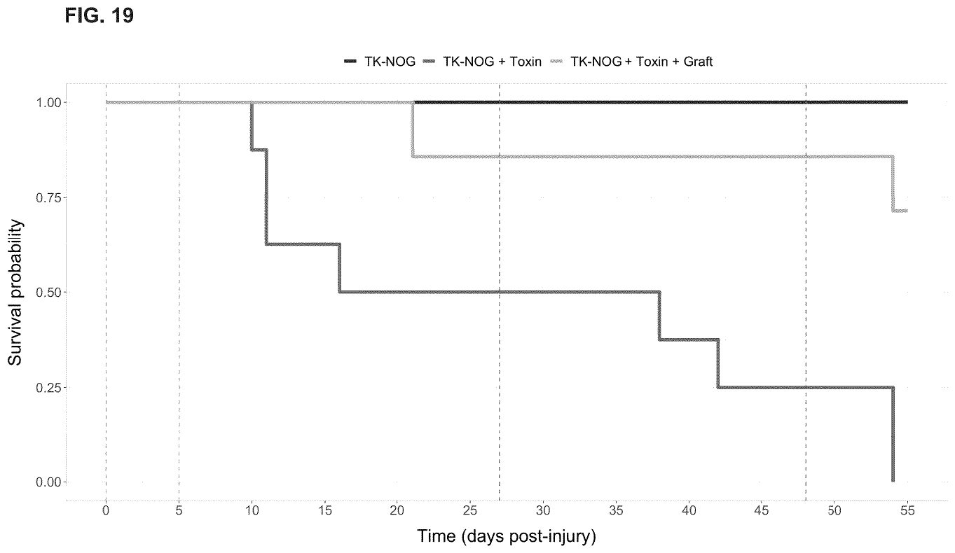

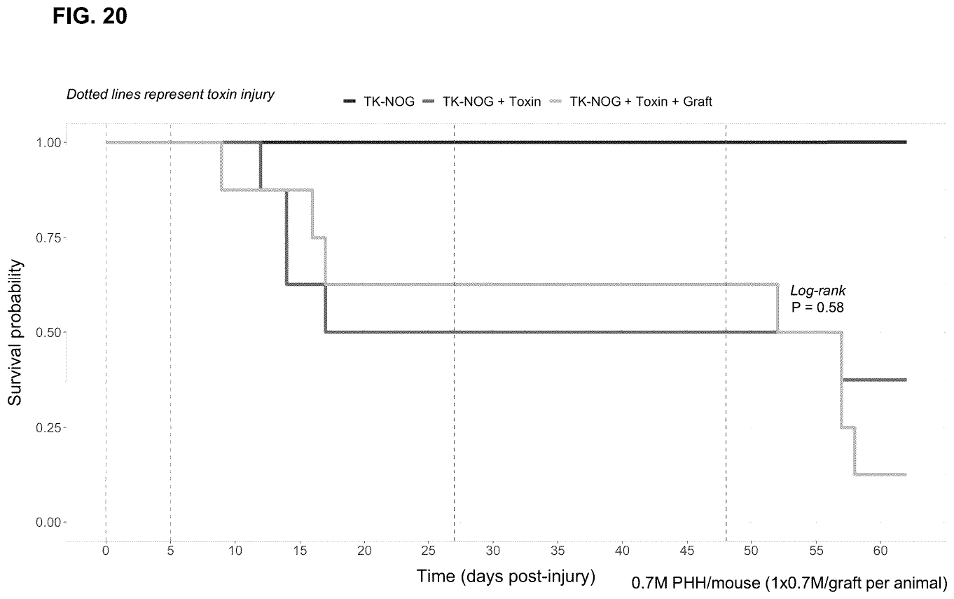

In evaluation of the levels of liver enzymes including alkaline phosphatase (ALP), alanine aminotransferase (ALT), and aspartate aminotransferase (AST), we observed that implantation of the high dose engineered tissue construct effectively normalized the levels of ALP, ALT, and AST, while the low dose engineered tissue construct elicited more modest effects (FIG. 18). In a probability of survival study, we observed that mice that were treated with GCV and implanted with a high-dose engineered tissue construct, exhibited an elevated probability to survive, whereas mice that received GCV alone, without the implantation of an engineered tissue construct, had a reduced probability to survive, with a sharp increase in mortality beginning on day 10 (FIG. 19). The low-dose engineered tissue construct was less effective in attenuating mortality (FIG. 20).

### Example 11. In Vivo Evaluation of Engineered Tissue Constructs in Immunocompetent Mice

To determine the effectiveness of engineered tissue constructs in an in vivo model of a urea cycle disorder, a study was performed with hypomorph transgenic mice having the sparse fur-abnormal skin and hair mutation (Otc^{spf-ash}) on the X chromosome. The Oto^{spf-ash} mutation results in the reduction of ornithine transcarbamylase (OTC), a critical enzyme in the urea cycle, activity in the liver. The reduction of hepatic OTC activity in this mouse model is usually from 5-10% compared to wild-type mice.

Otc^{spf-ash} transgenic mice were administered an immunosuppressant composition every two days. The immunosuppressant composition was administered every two days for six days prior to Day 1 of the study. One day before the start of the study, a blood draw was performed to obtain a baseline plasma ammonia concentration measurement. At Day 0, one group was implanted with two engineered tissue constructs, each consisting of 1.41×10⁶ PHH and 2.82×10⁶ NHDF, whilst the other was not (no-surgery control mice). At Week 1, the first of two NH4CI challenges was performed. 7.5 mmol/kg of NH4CI was administered to both groups (Otc^{spf-ash} no-surgery control group and Otc^{spf-ash}+graft) via the i.p. route. The day prior to each NH4CI challenge, all animals had their bladders emptied, the urine was discarded, and an overnight fast was initiated. Blood samples (50 μl) were collected before each NH4CI challenge, 20 minutes after, and 40 minutes after each NH4CI challenge. The other NH4CI challenge was performed Week 4.

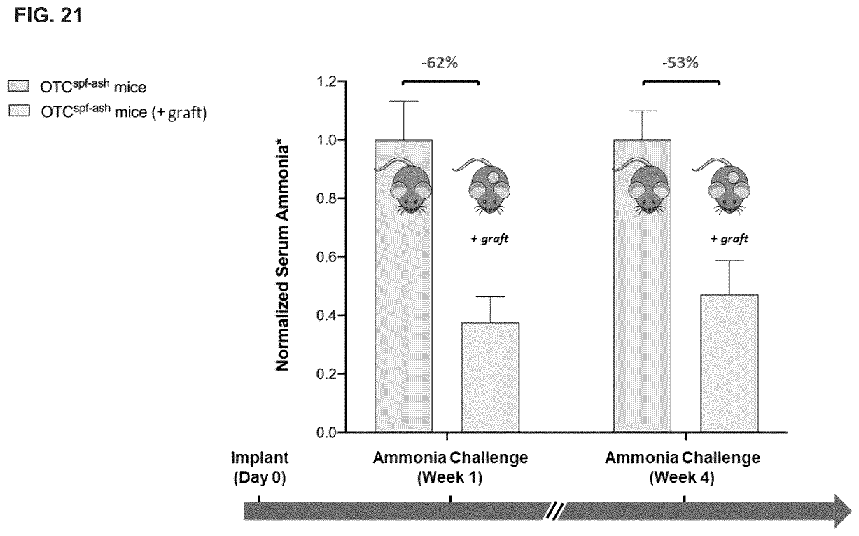

Otc^{spf-ash} that were implanted with the engineered tissue construct showed significant resiliency to the ammonia challenges compared to Otc^{spf-ash} no-surgery control group without an engineered tissue construct (FIG. 21).

### Example 12. Biomarker Development for Crigler-Najjar Syndrome Using Gunn Rat Model

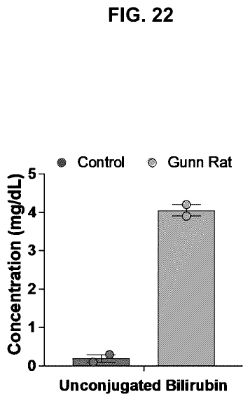

To determine whether unconjugated bilirubin could be used as a biomarker for Crigler-Najjar syndrome (e.g., Crigler-Najjar syndrome type I), the serum levels of unconjugated bilirubin were measured in homozygous Gunn rats and compared to the serum levels of unconjugated bilirubin in control Wistar rats. Gunn rats exhibited a minimum 4-fold increase of serum levels of unconjugated bilirubin compared to control Wistar rats, thereby establishing unconjugated bilirubin as a potential biomarker for Crigler-Najjar syndrome (FIG. 22).

### Example 13. Assessment of Albumin and Unconjugated Bilirubin in Gunn Rats

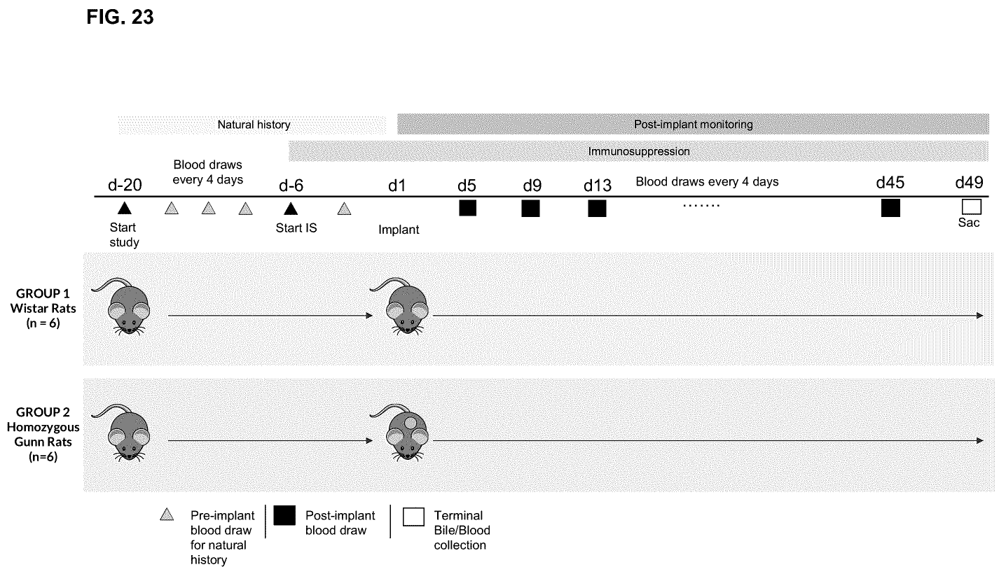

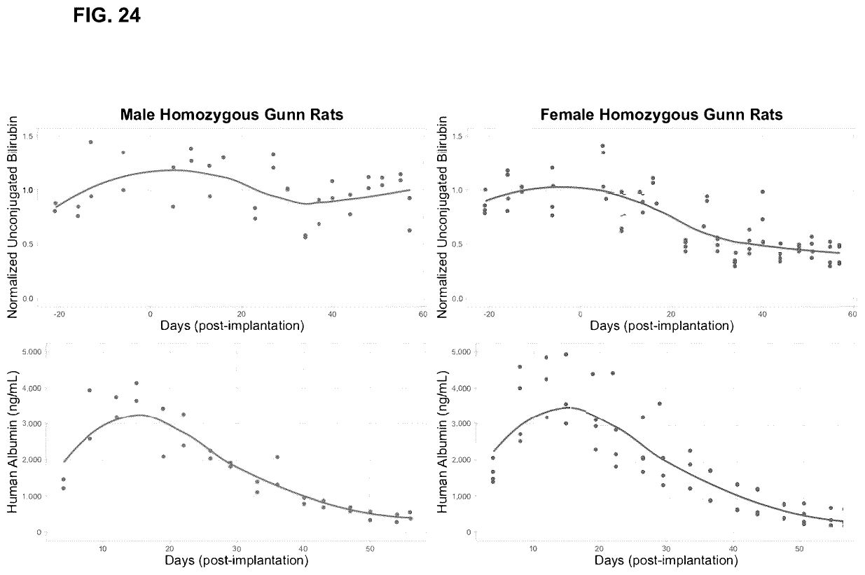

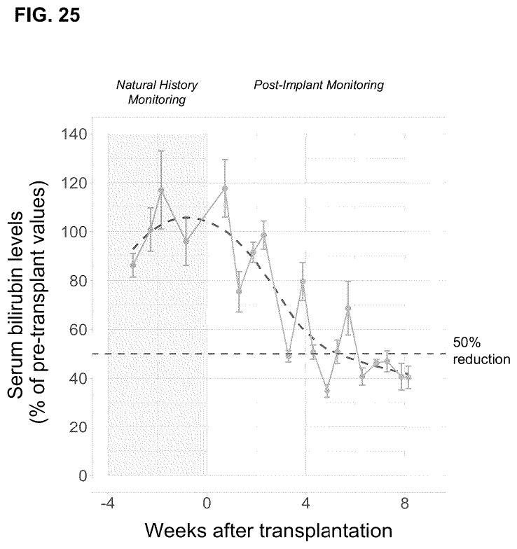

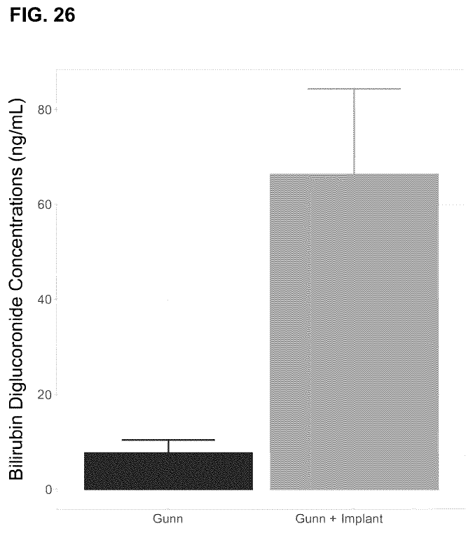

Homozygous Gunn rats were implanted with eight engineered tissue constructs including 1.41×10⁶ PHH and 2.82×10⁶ NHDF. Serum levels of unconjugated bilirubin and albumin were assessed by blood draw every fourth or fifth day, beginning from 20 days (p-20) pre-implantation to 49 days (d49) post-implantation (FIG. 23). Data were separated by sex and unconjugated bilirubin levels were normalized to control Wistar rats. Both unconjugated bilirubin and albumin serum levels decreased in homozygous Gunn rats post-implantation, regardless of sex (FIG. 24). Serum unconjugated bilirubin levels reduced by 50% within 5 weeks post-implantation (FIG. 25). Additionally, the presence of conjugated bilirubin products (bilirubin diglucoronide) in the bile was assessed in rats implanted with eight engineered tissue constructs, as compared to those without an engineered tissue constructs. Homozygous Gunn rats exhibited an increase in levels of conjugated bilirubin products in the bile relative to control Wistar rats (FIG. 26), confirming that the engineered tissue constructs of hepatocyte grafts partially rescued liver function by improving efficiency of bilirubin conjugation.

### Example 14. In Vivo Evaluation of Engineered Tissue Constructs in Immunocompetent Swine

To determine the effectiveness of engineered tissue constructs in an in vivo model, a study was performed with immunocompetent Yorkshire swine.

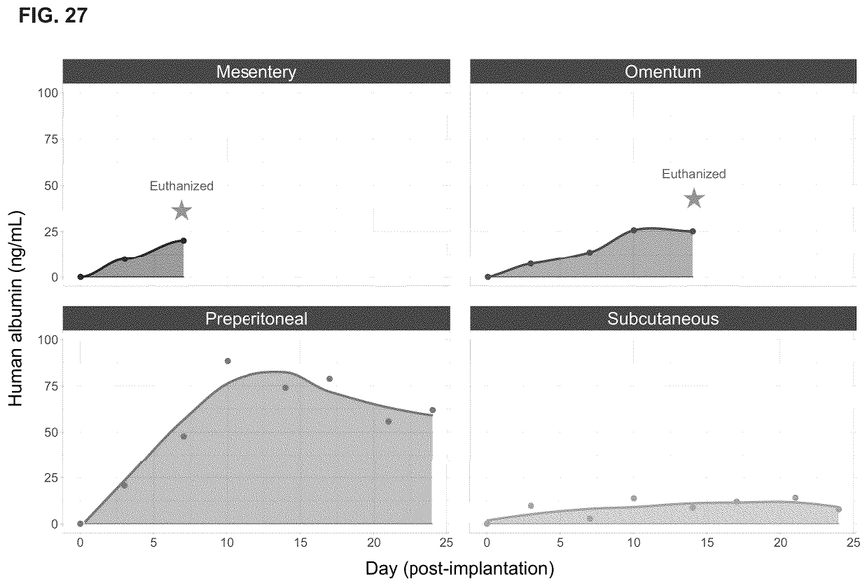

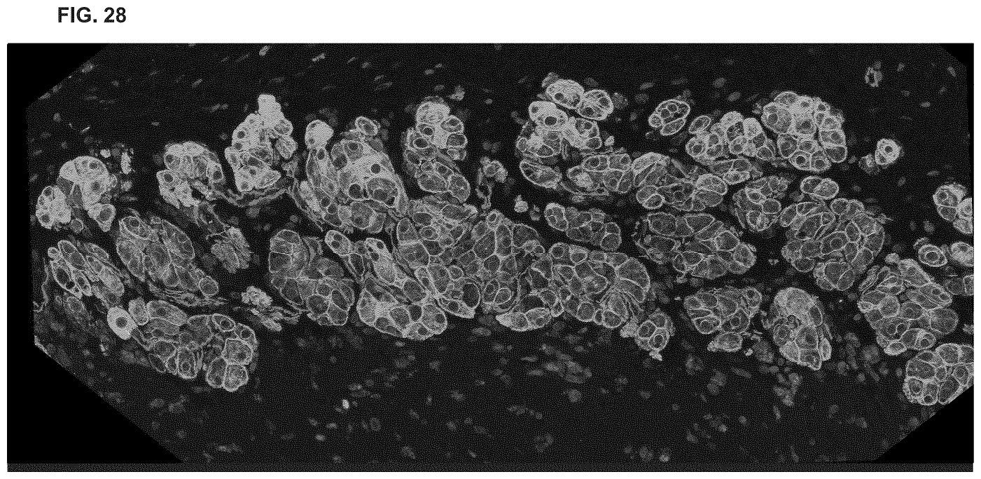

Swine implanted with engineered tissue constructs at the mesentery, omentum, preperitoneally, or subcutaneously, respectively, showed elevated levels of human albumin (ng/ml), with the preperitoneal implantation site eliciting the strongest expression of human albumin (FIG. 27). Following triggered euthanasia, histological analyses of the explanted engineered tissue constructs revealed functioning expression of hepatocytes and host blood vessels in the graft region (FIG. 28). Taken together, these histological analyses revealed a large number of hepatocytes that survived (e.g., persisted).

### Example 15. Methods of Making an Engineered Tissue Construct

An engineered tissue construct suitable for implantation into a subject is made with a plurality of spheroids that include hepatocytes (e.g., PHH) and, optionally, stromal cells (e.g., fibroblasts e.g., NHDF or neonatal foreskin fibroblasts) in a biocompatible scaffold (e.g., fibrin). The engineered tissue construct may also include a reinforcing agent (e.g., fibrin, surgical mesh, alginate, collagen, poly(ethylene glycol), polyvinylidene acetate, polyvinylidene fluoride, poly(lactic-co-glycolic) acid, or poly (I-lactic acid)).

The population of hepatocytes may be from 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 1.8×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5× 10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) cell. The optional population of stromal cells may be between 0 to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², 3×10⁶ to about 1.8×10¹², 4×10⁶ to about 1.8×10¹², 5×10⁶ to about 1.8×10¹², 6×10⁶ to about 1.8×10¹², 7×10⁶ to about 1.8×10¹², 8×10⁶ to about 1.8×10¹², 9×10⁶ to about 1.8×10¹², from about 1×10⁷ to about 1.8×10¹², from about 2×10⁷ to about 1.8×10¹², from about 18×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², or from about 1×10¹¹ to about 1.8×10¹²) cells.

The population of hepatocytes and the population of stromal cells, together, account for at least 70% (e.g., at least 71%, 72%, 73%, 74%, 75%, 80%, 85%, 90%, 95%, or 100%) of the total cells in the engineered tissue construct. In some embodiments, the engineered tissue construct further includes other cell types (e.g., endothelial cells e.g., up to 30% of the cells in the engineered tissue construct are endothelial cells). The density of the hepatocytes in the engineered tissue construct may be from, for example, 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). The ratio of hepatocytes to stromal cells may be between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

The biocompatible scaffold may have an x-axis, a y-axis, and a z-axis (e.g., the z-axis may be 500 μm to 5 mm (e.g., 600 μm to 4 mm, 700 μm to 3 mm, 800 μm to 2 mm, or 900 μm to 1 mm)). The hepatocytes and stromal cell aggregates are allowed to distribute non-homogenously along the z-axis of the biocompatible scaffold (e.g., by gravity), simultaneously during the step of encapsulation with, for example, a fibrinogen solution that is polymerized with thrombin. The aggregates may be distributed homogeneously in the x-axis and/or y-axis. The biocompatible scaffold polymerizes in less than 3 hours (e.g., less than 2 hours, 1 hour, 59 minutes, 58 minutes, 57 minutes, 56 minutes, 55 minutes, 50 minutes, 45 minutes, 40 minutes, 30 minutes, 20 minutes, 10 minutes, or 5 minutes. Furthermore, the engineered tissue construct may be from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

The layer may be from 100 μm to 2 mm (e.g., 200 μm to 1900 μm, 300 μm to 1800 μm, 400 μm to 1700 μm, 500 μm to 1600 μm, 600 μm to 1500 μm, 700 μm to 1400 μm, 800 μm to 1300 μm, 900 μm to 1200 μm, or 1000 μm to 1100 μm) thick. For example, the layer may be from 100 μm to 1 mm (e.g., 200 μm to 900 μm, 300 μm to 800 μm, 400 μm to 700 μm, or 500 μm to 600 μm) thick and/or the density of hepatocytes in the layer is from 0.06 M/cm² to 150 M/cm² (e.g., 0.07 M/cm² to 149 M/cm², 0.08 M/cm² to 148 M/cm², 0.09 M/cm² to 147 M/cm², 0.1 M/cm² to 146 M/cm², 0.2 M/cm² to 145 M/cm², 0.3 M/cm² to 140 M/cm², 0.4 M/cm² to 130 M/cm², 0.5 M/cm² to 120 M/cm², 1 M/cm² to 110 M/cm², 2 M/cm² to 100 M/cm², 3 M/cm² to 50 M/cm², 4 M/cm² to 40 M/cm², 5 M/cm² to 30 M/cm², or 10 M/cm² to 20 M/cm²). Furthermore, the ratio of height of the biocompatible scaffold to height of the layer may be from 20:1 to 1:1 e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1.

Afterwards, one or more engineered tissue constructs may be assembled with biocompatible glue (e.g., fibrin glue) such that each of the aggregate layers is outward facing, thereby forming a two-sided engineered tissue construct.

### Example 16. In Vitro Study for Measuring Seed Layer Height of Vertical Wheel Bioreactor Seeds

Due to the need to control seed layer height of the final graft for dose control, experiments assessing seed layer heights were performed. Vertical wheel bioreactor (VWB)-based seed layer heights trended differently than microwell based seed layer heights. It was assessed whether VWB seed layer heights generated from ROTEA™ washed NHDFs, PHHs, and seeds similar to non-ROTEAT washed and/or microwell seed layer heights at dose densities of 3, 6, and 9 M/mL

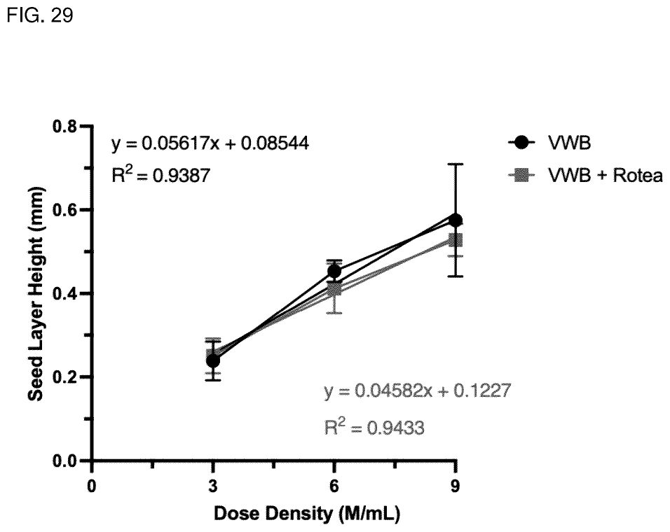

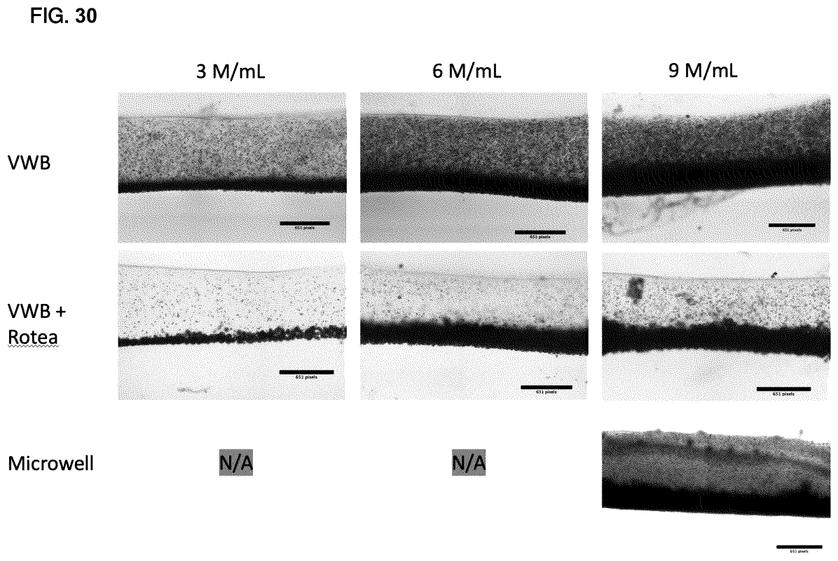

ROTEA™ washing NHDFs and PHHs post-thaw and seeds post-aggregation appears to yield on average slightly shorter seed layer heights compared to non-ROTEA™ washed seed layer heights at dose densities of 3, 6 and 9 M/mL (FIG. 29). T-test showed no significant difference between the two groups, indicating that the ROTEA™ washes did not impact the formation of seed layer heights. This also shows that the debris and seeds washed out during the ROTEA™ protocol were not the seeds that form the bulk of the seed layer height. This suggests that the ROTEA™ removed the cellular debris and smaller seeds that did not settle quick enough or the debris observed in the fibrin headspace in graft cross-sections. The clearer fibrin headspace allows for better distinguishing of the seed layer height as seen in FIG. 30.

In conclusion, these studies showed that ROTEA™ washed cells and seeds can be utilized without having a significant impact on seed layer height.

### Example 17. Impact of Seed Layer Thickness on Graft Function In Vivo

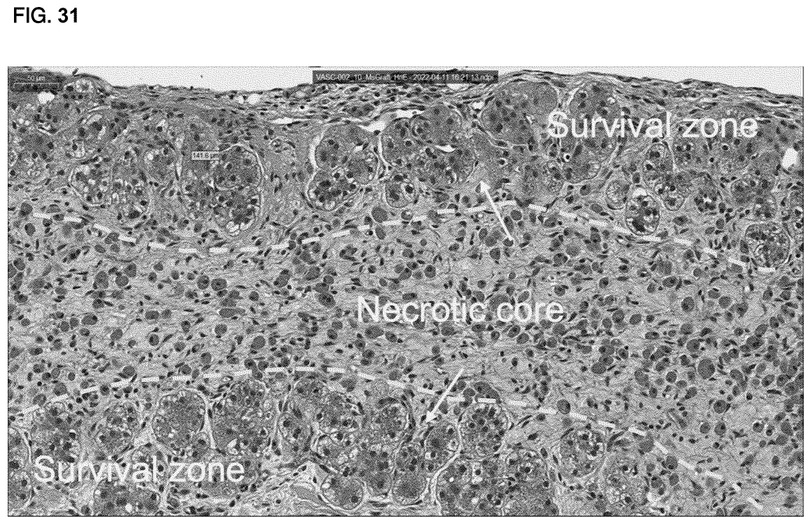

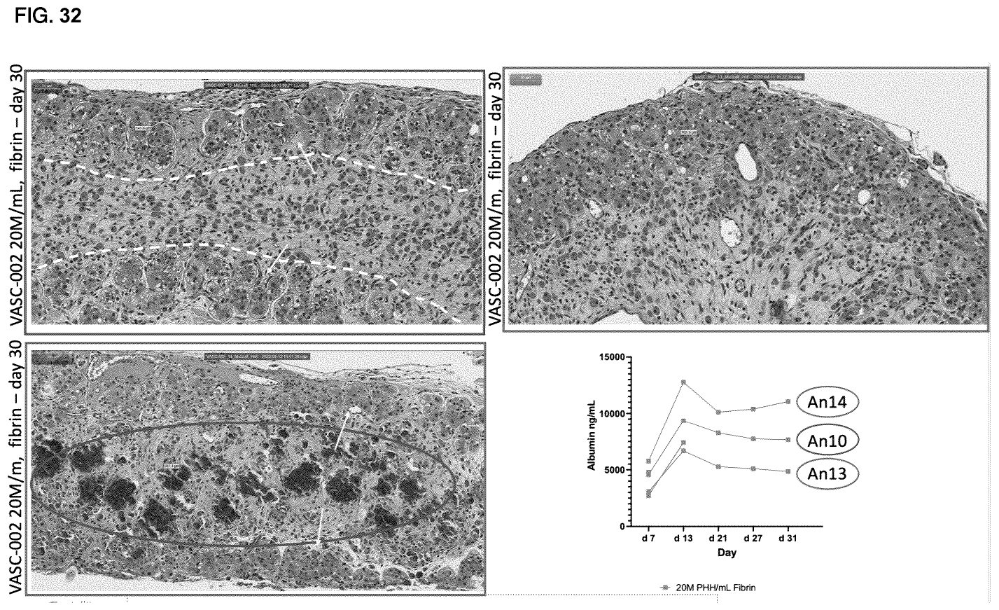

The impact of seed layer thickness on graft function was evaluated. Ultra-high density PHH grafts were formulated at 20 M PHH/ml in 7 mg/ml bovine fibrinogen and implanted on the fat pad of NSG mice. The grafts engrafted in vivo and exhibited bands of PHH with a necrotic core at Day 30 post-implantation (FIGS. 31 and 32). In FIG. 31, the survival zone thickness in terms of the number of seed diameters can be assessed. The survival zone may be occupied by 3 seeds thick, but more typically is about 2 seeds thick. At day 30, this corresponds to a thickness of 141 microns. These data indicate that the seed layer thickness in the pre-implant graft may be between 100 microns and 1 mm (e.g., between 300 microns and 500 microns).

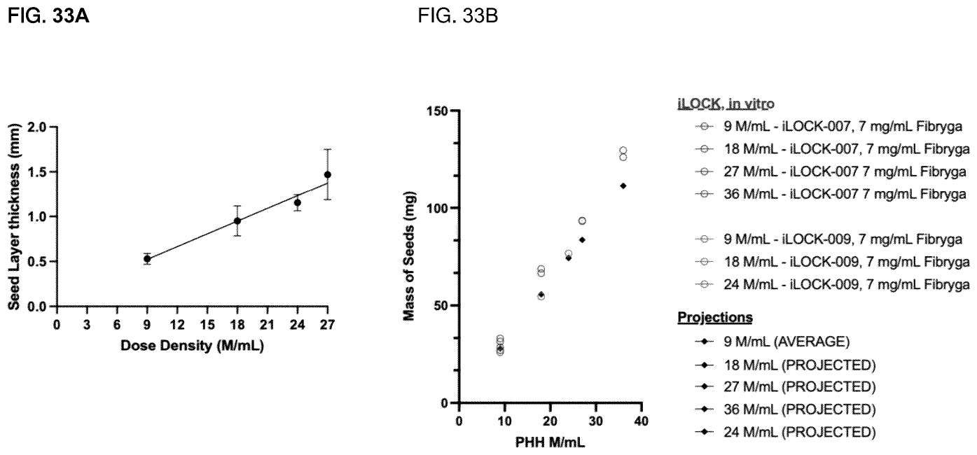

Production of albumin, a marker of engraftment, was also observed (FIG. 32), indicating that PHH engrafted in the fat pad. An13 exhibited only one band of integrated hepatocytes in the graft and showed the lowest albumin levels. An10 exhibited double layers of viable hepatocytes and showed higher albumin. An14 exhibited double layers of viable hepatocytes with a necrotic core and showed the highest level of albumin (FIG. 32). The dose density was examined to determine the impact on seed layer thickness and mass. Grafts were produced at various densities and seed layer thickness was measured. The dose density had a linear relationship with the seed layer thickness (FIG. 33A) and mass of seeds (FIG. 33B).

### Example 18. Biomarker Development for Crigler-Najjar Syndrome Using Gunn Rat Model

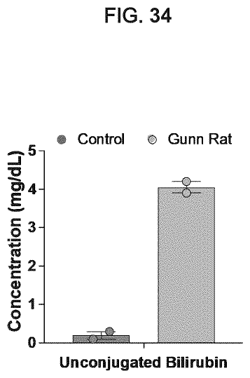

To determine whether unconjugated bilirubin could be used as a biomarker for Crigler-Najjar syndrome (e.g., Crigler-Najjar syndrome type I), the serum levels of unconjugated bilirubin were measured in homozygous Gunn rats and compared to the serum levels of unconjugated bilirubin in control Wistar rats. Gunn rats exhibited a minimum 4-fold increase of serum levels of unconjugated bilirubin compared to control Wistar rats, thereby establishing unconjugated bilirubin as a potential biomarker for Crigler-Najjar syndrome (FIG. 34).

### Example 19. Assessment of Albumin and Unconjugated Bilirubin in Gunn Rats

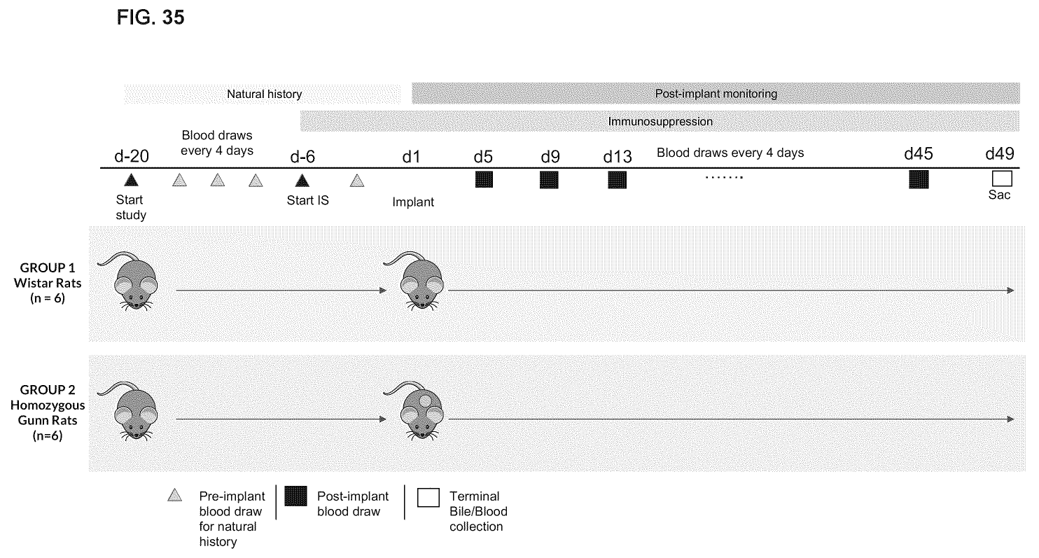

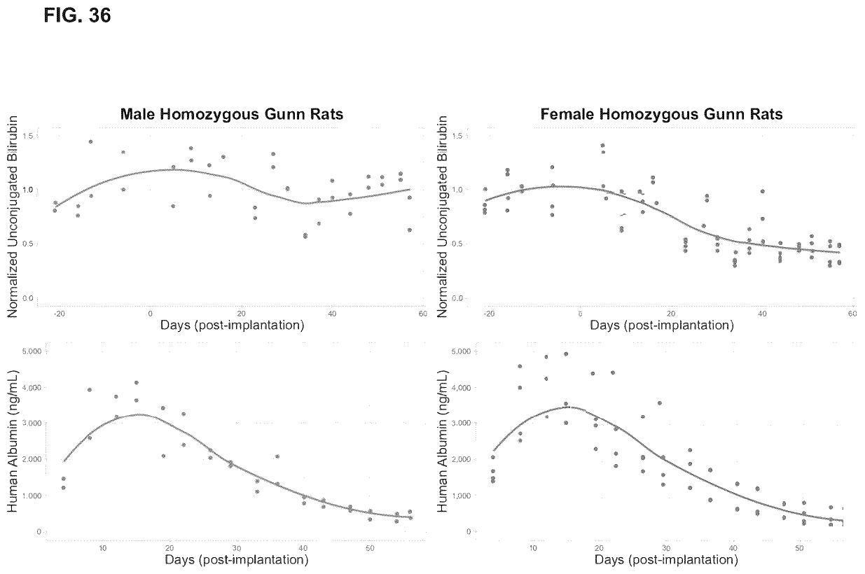

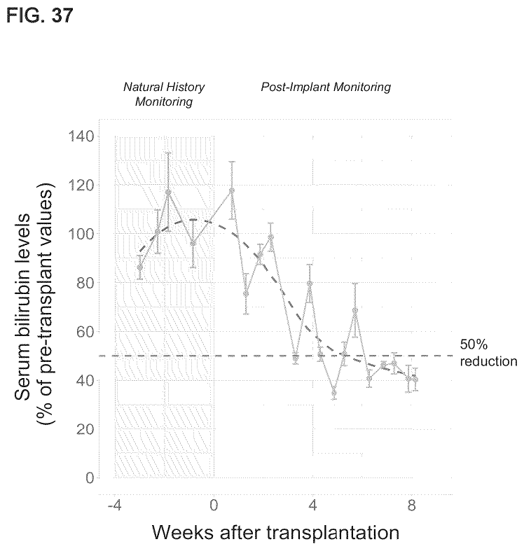

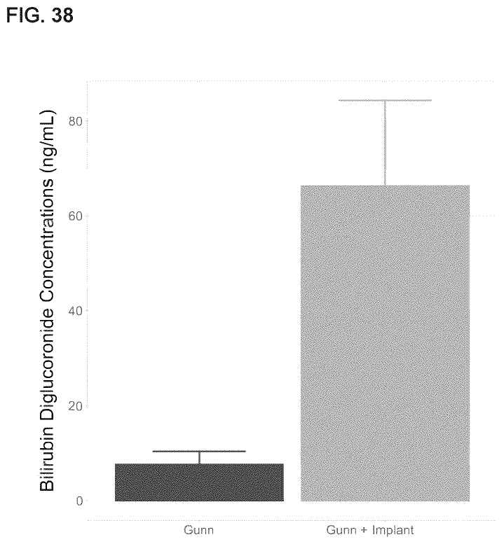

Homozygous Gunn rats were implanted with two implants including 1.41×10⁶ PHH and 2.82×10⁶ NHDF. Serum levels of unconjugated bilirubin and albumin were assessed by blood draw every fourth day, beginning from 20 days (p-20) pre-implantation to 49 days (d49) post-implantation (FIG. 35). Data were separated by sex and unconjugated bilirubin levels were normalized to control Wistar rats. Both unconjugated bilirubin and albumin serum levels decreased in homozygous Gunn rats post-implantation, regardless of sex (FIG. 36). Serum unconjugated bilirubin levels reduced by 50% within 5 weeks post-implantation (FIG. 37). Additionally, the presence of conjugated bilirubin products (bilirubin diglucuronide) in the bile was assessed in mice implanted with an implant, as compared to those without an implant. Homozygous Gunn rats exhibited an increase in levels of conjugated bilirubin products in the bile relative to control Wistar rats (FIG. 38), confirming that the implants of hepatocyte grafts partially rescued liver function by improving efficiency of bilirubin conjugation.

### Example 20. Treatment of Crigler-Najjar Syndrome Type II in a Human Patient by Implanting an Engineered Tissue Construct Including Hepatocytes and Stromal Cells

A pediatric patient (e.g., 1 year of age) having Crigler-Najjar syndrome type II is treated using an engineered tissue construct. After receiving a regimen of immunosuppressants, the patient is administered an engineered tissue construct that includes from about 2×10⁷ to about 6×10¹⁰ (e.g., from about 3×10⁷ to about 5×10¹⁰, from about 4×10⁷ to about 4×10¹⁰, or from about 4×10⁸ to about 4×10⁹) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. The patient's serum contains a level of total bilirubin that is less than or equal to about 1.2 mg/dl (e.g., less than or equal to about 1.1 mg/dl, 1.0 mg/dl, 0.9 mg/dl, 0.8 mg/dl, 0.7 mg/dl, 0.6 mg/dL, 0.5 mg/dl, 0.4 mg/dl, 0.3 mg/dL, or 0.2 mg/dL).

### Example 21. Treatment of Crigler-Najjar Syndrome Type I in a Human Patient by Implanting an Engineered Tissue Construct Including Hepatocytes and Stromal Cells

A neonate patient (age of 1 day old) having Crigler-Najjar syndrome type I is treated using an engineered tissue construct. After receiving a regimen of immunosuppressants, the patient is administered an engineered tissue construct that includes from about 3×10⁵ to about 3×10¹⁰ (e.g., 1×10⁶ to about 1×10¹⁰, or 1×10⁷ to about 1×10⁹, or about 1×10⁸) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. The patient's serum contains a level of direct bilirubin that is less than or equal to about 1.7 mg/dl (e.g., less than or equal to about 1.6 mg/dl, 1.5 mg/dl, 1.4 mg/dl, 1.3 mg/dl, 1.2 mg/dl, 1.1 mg/dl, 1.0 mg/dl, 0.9 mg/dL, 0.8 mg/dl, 0.7 mg/dl, 0.6 mg/dl, 0.5 mg/dl, 0.4 mg/dl, or 0.2 mg/dL).

### Example 22. Treatment of Crigler-Najjar Syndrome in a Human Patient by Implanting an Engineered Tissue Construct Including Hepatocytes and Stromal Cells

A patient (age of 18 years old) having Crigler-Najjar syndrome (e.g., Crigler-Najjar syndrome type II) is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. The patient's serum contains a level of bilirubin that is less than or equal to about 1 mg/dl (e.g., less than or equal to about 0.9 mg/dl, 0.8 mg/dl, 0.7 mg/dl, 0.6 mg/dl, 0.5 mg/dl, 0.4 mg/dL, 0.3 mg/dL, or 0.2 mg/dL).

### Ordered Embodiments

1. An engineered tissue construct suitable for implantation into a subject, the engineered tissue construct comprising a population of hepatocytes and a population of stromal cells, wherein the hepatocytes and stromal cells are in a biocompatible scaffold, and wherein the population of hepatocytes and the population of stromal cells together account for at least 90% of the total cells in the engineered tissue construct.
2. The engineered tissue construct of embodiment 1, wherein the engineered tissue construct further includes up to 10% of endothelial cells.
3. An engineered tissue construct suitable for implantation into a subject, the engineered tissue construct comprising a population of hepatocytes and a population of stromal cells, wherein the hepatocytes and stromal cells are in a biocompatible scaffold, and wherein the density of hepatocytes is 0.1 M/mL to 150 M/mL.
4. The engineered tissue construct of embodiment 3, wherein the density of hepatocytes is 0.5 M/mL to 25 M/mL.
5. The engineered tissue construct of embodiment 4, wherein the density of hepatocytes is 1 M/mL to 12 M/mL.
6. The engineered tissue construct of embodiment 5, wherein the density of hepatocytes is 9 M/mL.
7. An engineered tissue construct suitable for implantation into a subject, the engineered tissue construct comprising a population of hepatocytes and a population of stromal cells; wherein the hepatocytes and stromal cells are in a biocompatible scaffold; wherein the biocompatible scaffold has an x-axis, a y-axis, and a z-axis; and wherein the hepatocytes and stromal cells are distributed non-homogenously along the z-axis of the biocompatible scaffold.
8. An engineered tissue construct suitable for implantation into a subject, the engineered tissue construct comprising a plurality of spheroids in a biocompatible scaffold; wherein the spheroids comprise a population of hepatocytes and a population of stromal cells; wherein the biocompatible scaffold has an x-axis, a y-axis, and a z-axis; and wherein the spheroids are distributed non-homogenously, in a layer, along the z-axis of the biocompatible scaffold.
9. The engineered tissue construct of embodiment 8, wherein the layer is from 100 μm to 2 mm thick.
10. The engineered tissue construct of embodiment 8 or 9, wherein the density of hepatocytes in the layer is 0.06 M/cm² to 150 M/cm².
11. The engineered tissue construct of any one of embodiments 8-10, where the ratio of height of the biocompatible scaffold to height of the layer is from 20:1 to 1:1.
12. The engineered tissue construct of embodiment 11, wherein the ratio of height of the biocompatible scaffold to height of the layer is 2:1.
13. The engineered tissue construct of any one of embodiments 1-6, wherein the biocompatible scaffold has an x-axis, a y-axis, and a z-axis.
14. The engineered tissue construct of any one of embodiments 7-13, wherein the z-axis of the biocompatible scaffold is from 500 μm to 5 mm.
15. The engineered tissue construct of embodiment 14, wherein the z-axis of the biocompatible scaffold is 2 mm.
16. The engineered tissue construct of any one of embodiments 1-15, wherein the population of hepatocytes includes 3×10⁵ to 1.8×10¹¹ hepatocytes.
17. The engineered tissue construct of any one of embodiments 1-16, wherein the population of stromal cells includes up to 1.8×10¹² stromal cells.
18. The engineered tissue construct of any one of embodiments 1-17, wherein the ratio of hepatocytes to stromal cells is between 1:10 and 4:1.
19. The engineered tissue construct of any one of embodiments 1-18, wherein the hepatocytes are primary human hepatocytes.
20. The engineered tissue construct of any one of embodiments 1-19, wherein the stromal cells are fibroblasts.
21. The engineered tissue construct of embodiment 20, wherein the fibroblasts are selected from the group consisting of normal human dermal fibroblasts and neonatal foreskin fibroblasts.
22. The engineered tissue construct of embodiment 21, wherein the fibroblasts are neonatal foreskin fibroblasts.
23. The engineered tissue construct of any one of embodiments 1-22, wherein the engineered tissue construct is from 0.1 mL to 5 L in volume.
24. The engineered tissue construct of any one of embodiments 1, 2, or 7-23, wherein the density of hepatocytes is 0.1 M/mL to 150 M/mL.
25. The engineered tissue construct of embodiment 24, wherein the density of hepatocytes is 0.5 M/mL to 25 M/mL.
26. The engineered tissue construct of embodiment 25, wherein the density of hepatocytes is 1 M/mL to 12 M/mL.
27. The engineered tissue construct of embodiment 26, wherein the density of hepatocytes is 9 M/mL.
28. The engineered tissue construct of any one of embodiments 1-27, wherein the biocompatible scaffold is fibrin.
29. The engineered tissue construct of any one of embodiments 1-28, wherein the engineered tissue construct further includes a reinforcing agent.
30. The engineered tissue construct of embodiment 29, wherein the reinforcing agent is selected from the list comprising fibrin, surgical mesh, alginate, collagen, poly(ethylene glycol), polyvinylidene acetate (PVDA), polyvinylidene fluoride (PVDF), poly(lactic-co-glycolic) acid (PLGA), and poly (I-lactic acid) (PLLA).
31. The engineered tissue construct of any one of embodiments 1-30, wherein the engineered tissue construct has an ammonia clearance rate of at least 0.46 fmol/min/cell.
32. The engineered tissue construct of embodiment 31, wherein the engineered tissue construct has an ammonia clearance rate of at least 0.5 fmol/min/cell.
33. The engineered tissue construct of embodiment 32, wherein the engineered tissue construct has an ammonia clearance rate of at least 1 fmol/min/cell.
34. The engineered tissue construct of embodiment 33, wherein the engineered tissue construct has an ammonia clearance rate of at least 1.5 fmol/min/cell.
35. A kit comprising the engineered tissue construct of any one of embodiments 1-34, wherein the kit further includes a package insert instructing a user of the kit to implant the engineered tissue construct.
36. A method of making the engineered tissue construct of any one of embodiments 1-34,
wherein the population of hepatocytes and the population of stromal cells are aggregated in spheroids;
wherein the aggregated spheroids are at least partially embedded in the biocompatible scaffold;
wherein the biocompatible scaffold has an x-axis, a y-axis, and a z-axis; and
wherein the spheroids are allowed to non-homogenously distribute along the z-axis of the biocompatible scaffold into a layer, thereby forming the engineered tissue construct suitable for implantation in a subject.
37. A method of making the engineered tissue construct of any one of embodiments 1-34,
wherein the population of hepatocytes and the population of stromal cells are aggregated in spheroids;
wherein the aggregated spheroids are at least partially embedded in the biocompatible scaffold;
wherein the biocompatible scaffold has an x-axis, a y-axis, and a z-axis;
wherein the spheroids are allowed to non-homogenously distribute along the z-axis of the biocompatible scaffold into a layer, thereby forming one or more of a one-sided engineered tissue construct; and
wherein two or more of the one-sided engineered tissue constructs are assembled with each of the layers facing outwardly, thereby forming the engineered tissue construct suitable for implantation in a subject.
38. The method of embodiment 36 or 37, wherein the biocompatible scaffold polymerizes in less than 3 hours.
39. The method of embodiment 38, wherein the biocompatible scaffold polymerizes in 45 minutes or less.
40. The method of any one of embodiments 36-39, wherein the layer is from 100 μm to 2 mm thick.
41. The method of any one of embodiments 36-40, wherein the density of hepatocytes in the layer is from 0.06 M/cm² to 150 M/cm².
42. The method of any one of embodiments 36-41, where the ratio of height of the biocompatible scaffold to height of the layer is from 20:1 to 1:1.
43. The method of embodiment 42, wherein the ratio of height of the biocompatible scaffold to height of the layer is 2:1.
44. The method of any one of embodiments 36-43, wherein the z-axis of the biocompatible scaffold is from 500 μm to 5 mm.
45. The method of embodiment 44, wherein the z-axis of the biocompatible scaffold is 2 mm.
46. The method of any one of embodiments 36-45, wherein the population of hepatocytes includes 3×10⁵ to 1.8×10¹¹ hepatocytes.
47. The method of any one of embodiments 36-46, wherein the population of stromal cells includes up to 1.8×10¹² stromal cells.
48. The method of any one of embodiments 36-47, wherein the ratio of hepatocytes to stromal cells is between 1:10 and 4:1.
49. The method of any one of embodiments 36-48, wherein the hepatocytes are primary human hepatocytes.
50. The method of any one of embodiments 36-49, wherein the stromal cells are fibroblasts.
51. The method of embodiment 50, wherein the fibroblasts are selected from the group consisting of normal human dermal fibroblasts and neonatal foreskin fibroblasts.
52. The method of any one of embodiments 36-51, wherein the engineered tissue construct is from 0.1 mL to 5 L in volume.
53. The method of any one of embodiments 36-52, wherein the density of hepatocytes is 0.1 M/mL to 150 M/mL.
54. The method of any one of embodiments 36-53, wherein the biocompatible scaffold includes fibrin.
55. The method of any one of embodiments 36-54, wherein the engineered tissue construct further includes a reinforcing agent.
56. The method of embodiment 55, wherein the reinforcing agent is selected from the list comprising fibrin, surgical mesh, alginate, collagen, poly(ethylene glycol), PVDA, PVDF, PLGA, and PLLA.
57. A method of treating acute liver failure, a urea cycle disorder, or Crigler-Najjar syndrome in a human subject in need thereof, the method comprising implanting an engineered tissue construct comprising a population of hepatocytes and a population of stromal cells, wherein the hepatocytes and stromal cells are in a biocompatible scaffold, and wherein the population of hepatocytes and the population of stromal cells together account for at least 90% of the total cells in the engineered tissue construct.
58. The method of embodiment 57, wherein the engineered tissue construct further includes up to 10% of endothelial cells.
59. A method of treating acute liver failure, a urea cycle disorder, or Crigler-Najjar syndrome in a human subject in need thereof, the method comprising implanting an engineered tissue construct comprising a population of hepatocytes and a population of stromal cells, wherein the hepatocytes and stromal cells are in a biocompatible scaffold, and wherein the density of hepatocytes is 0.1 M/mL to 150 M/mL.
60. The method of embodiment 59, wherein the density of hepatocytes is 0.5 M/mL to 25 M/mL.
61. The method of embodiment 60, wherein the density of hepatocytes is 1 M/mL to 12 M/mL.
62. The method of embodiment 61, wherein the density of hepatocytes is 9 M/mL.
63. A method of treating acute liver failure, a urea cycle disorder, or Crigler-Najjar syndrome in a human subject in need thereof, the method comprising implanting one or more engineered tissue constructs comprising a population of hepatocytes and a population of stromal cells; wherein the hepatocytes and stromal cells are in a biocompatible scaffold; wherein the biocompatible scaffold has an x-axis, a y-axis, and a z-axis; and wherein the hepatocytes and stromal cells are distributed non-homogenously along the z-axis of the biocompatible scaffold.
64. A method of treating acute liver failure, a urea cycle disorder, or Crigler-Najjar syndrome in a human subject in need thereof, the method comprising implanting one or more engineered tissue constructs comprising a plurality of spheroids in a biocompatible scaffold; wherein the spheroids comprise a population of hepatocytes and a population of stromal cells; wherein the biocompatible scaffold has an x-axis, a y-axis, and a z-axis; and wherein the spheroids are distributed non-homogenously, in a layer, along the z-axis of the biocompatible scaffold.
65. The method of embodiment 64, wherein the layer is from 100 μm to 2 mm thick.
66. The method of embodiment 64 or 65, wherein the density of hepatocytes in the layer is 0.06 M/cm² to 150 M/cm².
67. The method of any one of embodiments 64-66, where the ratio of height of the biocompatible scaffold to height of the layer is from 20:1 to 1:1.
68. The method of embodiment 67, wherein the ratio of height of the biocompatible scaffold to height of the layer is 2:1.
69. The method of any one of embodiments 57-62, wherein the biocompatible scaffold has an x-axis, a y-axis, and a z-axis.
70. The method of any one of embodiments 63-69, wherein the z-axis of the biocompatible scaffold is from 500 μm to 5 mm.
71. The method of embodiment 70, wherein the z-axis of the biocompatible scaffold is 2 mm.
72. The method of any one of embodiments 57-71, wherein the population of hepatocytes includes 3×10⁵ to 1.8×10¹¹ hepatocytes.
73. The method of any one of embodiments 57-72, wherein the population of stromal cells includes up to 1.8×10¹² stromal cells.
74. The method of any one of embodiments 57-73, wherein the ratio of hepatocytes to stromal cells is between 1:10 and 4:1.
75. The method of any one of embodiments 57-74, wherein the hepatocytes are primary human hepatocytes.
76. The method of any one of embodiments 57-75, wherein the stromal cells are fibroblasts.
77. The method of embodiment 76, wherein the fibroblasts are selected from the group consisting of normal human dermal fibroblasts and neonatal foreskin fibroblasts.
78. The method of embodiment 77, wherein the fibroblasts are neonatal foreskin fibroblasts.
79. The engineered tissue construct of any one of embodiments 57-78, wherein the engineered tissue construct is from 0.1 mL to 5 L in volume.
80. The method of any one of embodiments 57, 58, or 63-79, wherein the density of hepatocytes is 0.1 M/mL to 150 M/mL.
81. The method of embodiment 80, wherein the density of hepatocytes is 0.5 M/mL to 25 M/mL.
82. The method of embodiment 81, wherein the density of hepatocytes is 1 M/mL to 12 M/mL.
83. The method of embodiment 82, wherein the density of hepatocytes is 9 M/mL.
84. The method of any one of embodiments 57-83, wherein the biocompatible scaffold is fibrin.
85. The method of any one of embodiments 57-84, wherein the one or more engineered tissue constructs further includes a reinforcing agent.
86. The method of embodiment 85, wherein the reinforcing agent is selected from the list comprising fibrin, surgical mesh, alginate, collagen, poly(ethylene glycol), PVDA, PVDF, PLGA, and PLLA.
87. The method of any one of embodiments 57-86, wherein the one or more engineered tissue constructs together have an ammonia clearance rate that is at least 200 μmol/min.
88. The method of embodiment 87, wherein the one or more engineered tissue constructs together have an ammonia clearance rate that is at least 300 μmol/min.
89. The method of embodiment 88, wherein the one or more engineered tissue construct together have an ammonia clearance rate that is at least 400 μmol/min.
90. The method of embodiment 89, wherein the one or more engineered tissue constructs together have an ammonia clearance rate that is at least 500 μmol/min.
91. The method of any one of embodiments 57-90, wherein the one or more engineered tissue constructs is implanted into the subject at an implantation site selected from the group consisting of the peritoneum, peritoneal cavity, rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization.
92. The method of embodiment 91, wherein the peritoneum is the retroperitoneum.
93. The method of embodiment 91, wherein the peritoneal cavity is the omentum or the mesentery.
94. The method of embodiment 93, wherein the omentum is the greater omentum or the omental bursa.
95. The method of embodiment 93, wherein the mesentery is the small intestinal mesentery.
96. The method of embodiment 91, wherein the implantation site is an extraperitoneal site.
97. The method of embodiment 91, wherein the implantation site is a site on the surface of the liver.
98. The method of embodiment 91, wherein the implantation site is an extrapleural site.
99. The method of embodiment 91, wherein the implantation site is a site that is suitable for neovascularization.
100. The method of any one of embodiments 57-99, wherein the one or more engineered tissue constructs is implanted into the subject at an implantation site that has a microvessel density of greater than about 3.6 vessels/mm².
101. The method of any one of embodiments 57-100, wherein following implantation of the one or more engineered tissue constructs, the subject exhibits a level of serum ammonia of less than or equal to about 90 μmol/L.
102. The method of any one of embodiments 57-101, wherein following implantation of the one or more engineered tissue constructs, the subject exhibits a change in one or more parameters in a blood test relative to a reference level.
103. The method of embodiment 102, wherein the blood test is a liver function test.
104. The method of embodiment 102 or 10³, wherein the one or more parameters includes the level of gamma-glutamyl transferase, alkaline phosphatase, aspartate aminotransferase, alanine aminotransferase, or albumin.
105. The method of any one of embodiments 57-10⁴, wherein following implantation of the one or more engineered tissue constructs, the subject exhibits an improvement in a test of gallbladder ejection fraction.
106. The method of embodiment 10⁵, wherein the test is a hepatobiliary iminodiacetic acid scan.
10⁷. The method of any one of embodiments 57-10⁶, wherein following implantation of the one or more engineered tissue constructs, the subject exhibits a level of total bilirubin of less than or equal to about 1.2 mg/dL.
108. The method of any one of embodiments 57-10⁷, wherein following implantation of the one or more engineered tissue constructs, the subject exhibits a level of direct bilirubin of less than or equal to about 1.7 mg/dL.
109. The method of any one of embodiments 57-10⁸, wherein following implantation of the one or more engineered tissue constructs, the subject exhibits a level of bilirubin of less than or equal to about 1 mg/dL.
110. The method of any one of embodiments 57-10⁹, wherein following implantation of the one or more engineered tissue constructs, the engineered tissue construct persists for at least two weeks.
111. The method of embodiment 110, wherein following implantation of the one or more engineered tissue construct, the engineered tissue construct persists for at least one month.
112. The method of embodiment 111, wherein following implantation of the one or more engineered tissue construct, the engineered tissue construct persists for at least five months.
113. The method of any one of embodiments 57-112, wherein the subject in need thereof has Crigler-Najjar syndrome.
114. The method of embodiment 112, wherein the Crigler-Najjar syndrome is Crigler-Najjar syndrome type I or Crigler-Najjar syndrome type II.
115. The method of any one of embodiments 57-114, wherein the subject in need thereof has a urea cycle disorder.
116. The method of any one of embodiments 57-114, wherein the subject in need thereof has acute liver failure.
117. The method of any one of embodiments 116, wherein the subject weighs less than 15 kg
118. The method of embodiment 117, wherein the subject weighs less than 10 kg.
119. The method of embodiment 118, wherein the subject weighs less than 5 kg.
120. The method of embodiment 119, wherein the subject weighs about 5 kg.
121. A kit comprising an engineered tissue construct, wherein the kit further includes a package insert instructing a user of the kit to implant the engineered tissue construct into the subject in accordance with the method of any one of embodiments 57-120.
122. A method of treating acute liver failure in a human subject in need thereof, the method comprising implanting an engineered tissue construct comprising a population of hepatocytes and a population of stromal cells wherein the engineered tissue construct provides a microenvironment that promotes the persistence of hepatocyte survival for at least three months in the subject.
123. The method of embodiment 122, wherein the population of hepatocytes includes an amount of hepatocytes that is equivalent to 0.5% to 30% of the total liver mass of the subject.
124. The method of embodiment 122, wherein the population of hepatocytes includes an amount of hepatocytes that is equivalent to 0.5% to 20% of the mass of the liver preserve of the subject.
125. The method of embodiment 122, wherein the population of hepatocytes includes 3×10⁵ to 1.8×10¹¹ hepatocytes.
126. The method of embodiment 122, wherein the population of stromal cells includes up to 1.8×10¹² stromal cells.
127. The method of any one of embodiments 122-12126, wherein the hepatocytes are primary human hepatocytes.
128. The method of any one of embodiments 122-127, wherein the stromal cells are fibroblasts.
129. The method of embodiment 128, wherein the fibroblasts are selected from the group consisting of normal human dermal fibroblasts and neonatal foreskin fibroblasts.
130. The method of embodiment 129, wherein the fibroblasts are neonatal foreskin fibroblasts.
131. The method of any one of embodiments 122-130, wherein the ratio of hepatocytes to stromal cells is between 1:10 and 4:1.
132. The method of any one of embodiments 122-131, wherein the engineered tissue construct is from 0.1 mL to 5 L in volume.
133. The method of embodiment 122 or 123, wherein hepatocytes are at a density of 0.1 M/mL to 150 M/mL.
134. The method of any one of embodiments 122-133, wherein the engineered tissue construct further includes a biocompatible hydrogel scaffold.
135. The method of embodiment 134, wherein the biocompatible scaffold comprises fibrin.
136. The method of any one of embodiments 1-135, wherein the engineered tissue construct is implanted into the subject at an implantation site selected from the group consisting of the peritoneum, peritoneal cavity, rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization.
137. The method of embodiment 136, where the peritoneum is the retroperitoneum.
138. The method of embodiment 136, wherein the peritoneal cavity is the omentum or the mesentery.
139. The method of embodiment 138, wherein the omentum is the greater omentum or the omental bursa.
140. The method of embodiment 138, wherein the mesentery is the small intestinal mesentery.
141. The method of embodiment 136, wherein the implantation site is an extraperitoneal site.
142. The method of embodiment 136, wherein the implantation site is a site on the surface of the liver.
143. The method of embodiment 136, wherein the implantation site is an extrapleural site.
144. The method of embodiment 136, wherein the implantation site is a site that is suitable for neovascularization.
145. The method of any one of embodiments 122-144, wherein the engineered tissue construct is implanted into the subject at an implantation site that has a microvessel density of greater than about 3.6 vessels/mm².
146. The method of any one of embodiments 122-145, wherein following implantation of the engineered tissue construct, the subject exhibits a level of serum ammonia of less than or equal to about 50 μmol/L.
147. The method of any one of embodiments 122-146, wherein following implantation of the engineered tissue construct, the subject exhibits a change in one or more parameters in a blood test relative to a reference level.
148. The method of embodiment 147, wherein the blood test is a liver function test.
149. The method of embodiment 147 or 148, wherein the one or more parameters includes the level of gamma-glutamyl transferase, alkaline phosphatase, aspartate aminotransferase, alanine aminotransferase, or albumin.
150. The method of any one of embodiments 122-149, wherein following implantation of the engineered tissue construct, the subject exhibits an improvement in a test of gallbladder ejection fraction.
151. The method of embodiment 150, wherein the test is a hepatobiliary iminodiacetic acid scan.
152. The method of any one of embodiments 122-151, wherein the human subject weighs less than 15 kg.
153. The method of embodiment 152, wherein the human subject weighs less than 10 kg.
154. The method of embodiment 153, wherein the human subject weighs less than 5 kg.
155. The method of embodiment 152, wherein the human subject weighs about 5 kg.
156. A kit comprising an engineered tissue construct, wherein the kit further includes a package insert instructing a user of the kit to implant the engineered tissue construct to the subject in accordance with the method of any one of embodiments 122-155.
157. A method of treating hyperbilirubinemia in a subject having Crigler-Najjar syndrome, the method comprising implanting one or more engineered tissue constructs comprising a population of hepatocytes and a population of stromal cells in amounts effective to treat hyperbilirubinemia in the subject.
158. A method of treating Crigler-Najjar syndrome in a subject in need thereof, the method comprising implanting one or more engineered tissue constructs comprising a population of hepatocytes and a population of stromal cells in amounts effective to treat the Crigler-Najjar syndrome.
159. A method of reducing bilirubin levels in a subject having Crigler-Najjar syndrome, the method comprising implanting one or more engineered tissue constructs comprising a population of hepatocytes and a population of stromal cells in amounts effective to reduce bilirubin levels in the subject.
160. The method of any one of embodiments 157-159, wherein the Crigler-Najjar syndrome is Crigler-Najjar syndrome type I or Crigler-Najjar syndrome type II.
161. The method of any one of embodiments 157-160, wherein the population of hepatocytes comprises an amount of hepatocytes that is equivalent to 0.5% to 30% of the total liver mass of the subject.
162. The method any one of embodiments 157-161, wherein the population of hepatocytes comprises an amount of hepatocytes that is equivalent to 0.5% to 20% of the mass of the liver reserve of the subject.
163. The method of any one of embodiments 157-162, wherein the population of hepatocytes comprises 3×10⁵ to 1.8×10¹¹ hepatocytes.
164. The method of any one of embodiments 157-163, wherein the population of stromal cells comprises 0 to 1.8×10¹² stromal cells.
165. The method of any one of embodiments 157-164, wherein the hepatocytes are primary human hepatocytes.
166. The method of any one of embodiments 157-165, wherein the stromal cells are fibroblasts.
167. The method of embodiment 166, wherein the fibroblasts are selected from the group consisting of normal human dermal fibroblasts and neonatal foreskin fibroblasts.
168. The method of embodiment 167 wherein the fibroblasts are neonatal foreskin fibroblasts.
169. The method of any one of embodiments 157-168, wherein the ratio of hepatocytes to stromal cells is between 1:10 and 4:1.
170. The method of any one of embodiments 157-169, wherein the engineered tissue construct is from 0.1 mL to 5 L in volume.
171. The method of any one of embodiments 157-170, wherein the density of hepatocytes is 0.1 M/mL to 150 M/mL.
172. The method of any one of embodiments 157-171, wherein the engineered tissue construct further comprises a biocompatible hydrogel scaffold.
173. The method of embodiment 172, wherein the biocompatible hydrogel scaffold comprises fibrin.
174. The method of any one of embodiments 157-173, wherein the engineered tissue construct is implanted into the subject at an implantation site selected from the group consisting of the peritoneum, peritoneal cavity, rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization.
175. The method of embodiment 174, wherein the peritoneum is the retroperitoneum.
176. The method of embodiment 174, wherein the peritoneal cavity is the omentum or the mesentery.
177. The method of embodiment 176, wherein the omentum is the greater omentum or the omental bursa.
178. The method of embodiment 177, wherein the mesentery is the small intestinal mesentery.
179. The method of embodiment 174, wherein the implantation site is an extraperitoneal site.
180. The method of embodiment 174, wherein the implantation site is a site on the surface of the liver.
181. The method of embodiment 174, wherein the implantation site is an extrapleural site.
182. The method of embodiment 174, wherein the implantation site is a site that is suitable for neovascularization.
183. The method of any one of embodiments 157-182, wherein the engineered tissue construct is implanted into the subject at an implantation site that has a microvessel density of greater than about 3.6 vessels/mm².
184. The method of any one of embodiments 157-183, wherein the subject is a human.
185. The method of any one of embodiments 157-184, wherein following implantation of the engineered tissue construct, the subject exhibits a level of total bilirubin of less than or equal to about 1.2 mg/dL.
186. The method of any one of embodiments 157-185, wherein following implantation of the engineered tissue construct, the subject exhibits a level of direct bilirubin of less than or equal to about 1.7 mg/dL.
187. The method of any one of embodiments 157-186, wherein following implantation of the engineered tissue construct, the subject exhibits a level of bilirubin of less than or equal to about 1 mg/dL.
188. A kit comprising an engineered tissue construct, wherein the kit further comprises a package insert instructing a user of the kit to implant the engineered tissue construct to the subject in accordance with the method of any one of embodiments 157-187.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating acute liver failure in a human subject in need thereof, the method comprising implanting an engineered tissue construct comprising a population of hepatocytes and a population of stromal cells wherein the engineered tissue construct provides a microenvironment that promotes the persistence of hepatocyte survival for at least three months in the subject.

2. The method of claim 1, wherein the population of hepatocytes includes an amount of hepatocytes that is equivalent to 0.5% to 30% of the total liver mass of the subject.

3. The method of claim 1, wherein the population of hepatocytes includes an amount of hepatocytes that is equivalent to 0.5% to 20% of the mass of the liver preserve of the subject.

4. The method of claim 1, wherein the population of hepatocytes includes 3×10⁵ to 1.8×10¹¹ hepatocytes.

5. The method of claim 1, wherein the population of stromal cells includes up to 1.8×10¹² stromal cells.

6. The method of any one of claims 1-5, wherein the hepatocytes are primary human hepatocytes.

7. The method of any one of claims 1-6, wherein the stromal cells are fibroblasts.

8. The method of claim 7, wherein the fibroblasts are selected from the group consisting of normal human dermal fibroblasts and neonatal foreskin fibroblasts.

9. The method of claim 8, wherein the fibroblasts are neonatal foreskin fibroblasts.

10. The method of any one of claims 1-9, wherein the ratio of hepatocytes to stromal cells is between 1:10 and 4:1.

11. The method of any one of claims 1-10, wherein the engineered tissue construct is from 0.1 mL to 5 L in volume.

12. The method of claim 1 or 2, wherein hepatocytes are at a density of 0.1 M/mL to 150 M/mL.

13. The method of any one of claims 1-12, wherein the engineered tissue construct further includes a biocompatible hydrogel scaffold.

14. The method of claim 13, wherein the biocompatible scaffold comprises fibrin.

15. The method of any one of claims 1-14, wherein the engineered tissue construct is implanted into the subject at an implantation site selected from the group consisting of the peritoneum, peritoneal cavity, rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization.

16. The method of claim 15, where the peritoneum is the retroperitoneum.

17. The method of claim 15, wherein the peritoneal cavity is the omentum or the mesentery.

18. The method of claim 17, wherein the omentum is the greater omentum or the omental bursa.

19. The method of claim 17, wherein the mesentery is the small intestinal mesentery.

20. The method of claim 15, wherein the implantation site is an extraperitoneal site.

21. The method of claim 15, wherein the implantation site is a site on the surface of the liver.

22. The method of claim 15, wherein the implantation site is an extrapleural site.

23. The method of claim 15, wherein the implantation site is a site that is suitable for neovascularization.

24. The method of any one of claims 1-23, wherein the engineered tissue construct is implanted into the subject at an implantation site that has a microvessel density of greater than about 3.6 vessels/mm².

25. The method of any one of claims 1-24, wherein following implantation of the engineered tissue construct, the subject exhibits a level of serum ammonia of less than or equal to about 50 μmol/L.

26. The method of any one of claims 1-25, wherein following implantation of the engineered tissue construct, the subject exhibits a change in one or more parameters in a blood test relative to a reference level.

27. The method of claim 26, wherein the blood test is a liver function test.

28. The method of claim 26 or 27, wherein the one or more parameters includes the level of gamma-glutamyl transferase, alkaline phosphatase, aspartate aminotransferase, alanine aminotransferase, or albumin.

29. The method of any one of claims 1-28, wherein following implantation of the engineered tissue construct, the subject exhibits an improvement in a test of gallbladder ejection fraction.

30. The method of claim 29, wherein the test is a hepatobiliary iminodiacetic acid scan.

31. The method of any one of claims 1-30, wherein the human subject weighs less than 15 kg.

32. The method of claim 31, wherein the human subject weighs less than 10 kg.

33. The method of claim 32, wherein the human subject weighs less than 5 kg.

34. The method of claim 31, wherein the human subject weighs about 5 kg.

35. A kit comprising an engineered tissue construct, wherein the kit further includes a package insert instructing a user of the kit to implant the engineered tissue construct to the subject in accordance with the method of any one of claims 1-34.
